(19) United States
(12) Patent Application Publication
(10) Pub. No.: US 2004/0022789 A1
(43) Pub. Date: Feb. 5, 2004

(54) Methods and compositions for treating T cell mediated inflammatory/autoimmune diseases and disorders in subjects having a glucocorticoid regulation deficiency

(75) Inventors: Judson A. Brewer, St. Louis, MO (US); Louis J. Muglia, Kirkwood, MO (US)

Correspondence Address: Charles E. Dunlap, Third Floor, Keenan Building, 1330 Lady Street, Columbia, SC, 29201, US

(73) Assignee: The Washington University, St. Louis, MO (US)

(21) Appl. No.: 10/427,683

(22) Filed: May 1, 2003

Related U.S. Application Data
(60) Provisional application No. 60/377,112, filed on May 2, 2002.
(60) Provisional application No. 60/381,188, filed on May 16, 2002.

(51) Int. Cl. A61K 39/395; A61K 31/415; A61K 31/365

(52) U.S. Cl. 424/146.1; 514/406; 514/473

(57) ABSTRACT

The present invention provides a method for preventing or treating a T cell mediated inflammatory/autoimmune disease or disorder in a subject having a glucocorticoid regulation deficiency, where the method comprises administering to a subject in need of such treatment a cyclooxygenase-2 inhibitor. The Cox-2 inhibitor may be administered in combination with a glucocorticoid. The Cox-2 inhibitor can be a Cox-2 selective inhibitor. Compositions, pharmaceutical compositions and kits are provided for carrying out the method.

### CROSS-REFERENCE TO RELATED PATENTS AND PATENT APPLICATIONS

This application claims priority to U.S. Provisional Application No. 60/377,112, filed May 2, 2002, and also to U.S. Provisional Application No. 60/381,188, filed May 16, 2002, each of which is incorporated by reference herein in its entirety.

### BACKGROUND OF THE INVENTION

(1) Field of the Invention

The present invention relates to methods and compositions for limiting morbidity and mortality arising from T cell activation in subjects having a glucocorticoid regulation deficiency, and more particularly to methods and compositions for preventing and treating T cell mediated inflammatory/autoimmune diseases and disorders in subjects having a glucocorticoid regulation deficiency.

(2) Description of the Related Art

T cells are lymphocytes that play a key role in the immune system. During normal immune system function in a vertebrate, the activation of T cells triggers the production of a number of inflammatory active molecules, including various cytokines and eicosanoids, including the prostaglandins. The presence of the cytokines in the hypothalamus and pituitary is known to cause the production of adrenocorticotropic hormone (ACTH, corticotropin), which acts on the adrenal gland to cause the production of glucocorticoids. The increased level of glucocorticoids, in turn, down-regulates the expression of the inflammatory cytokines in the T cells, thereby modulating the inflammatory immune response in the subject. Dysfunction of the feedback regulatory effect by glucocorticoids can cause morbidity and death. See, e.g., Webster, J. I. et al., Annu. Rev. Immunol., 20:125 - 163 (2002).

The direct or indirect administration of glucocorticoids (GCs) is a mainstay therapy for treatment of inflammatory conditions, autoimmune diseases, and lymphomas. Webster, J. I. et al., Annu. Rev. Immunol., 20:125 - 163 (2002). Consistent with these immunosuppressive effects of steroids, in vivo and in vitro studies have shown that pharmacologic levels of GCs, acting through the glucocorticoid receptor (GR), modulate cytokine synthesis and affect T cell and macrophage function. Almawi, W. Y. et al., J. Leukoc. Biol, 60:563 - 572 (1996). Additionally, physiologic GCs, which are released at high levels into the blood stream via cytokine activation of the hypothalamic-pituitary-adrenal (HPA) axis, have also been shown to be critical in maintaining homeostasis. Removal of systemic glucocorticoids via adrenalectomy in animal models or adrenal insufficiency in humans (as with Addison's disease) has demonstrated the requirement for endogenous GC production for regulation of physiologic immune responses. Bertini, R. et al., J. Exp. Med., 167:1708-1712 (1988). However, critical cellular and molecular targets of endogenous GR action in modulating physiological inflammatory responses remain unclear.

One important function of GR in maintaining normal homeostasis is its participation in modulation of the immune response of the HPA axis. In this negative-feedback loop, an adrenal gland-derived glucocorticoid (corticosterone in rodents, cortisol in humans) acts via the hypothalamus and pituitary to regulate its own production. Additionally, the HPA axis can be regulated by cytokines, neuropeptides and the sympathetic nervous system. Da Silva, J. A., Ann N Y. Acad. Sci., 876:102-117 (1999).

Glucocorticoids have also been shown to regulate expression of pro-inflammatory mediators in addition to cytokines. Of note, cyclooxygenase-2 (Cox-2) was discovered as a GC-modulated enzyme that was induced in monocytes after lipopolysaccharide (LPS) administration, and subsequently has been shown to be induced in vitro in T cells after activation. See, e.g., Iniguez, M. A. et al., J. Immunol, 163:111-119 (1999); Masferrer, J. L. et al., Proc. Natl. Acad. Sci. USA, 89:3917-3921 (1992).

In subjects wherein the glucocorticoid/glucocorticoid receptor regulatory mechanism does not function normally, immune system challenge results in the hyperproduction of cytokines and eicosanoids. The overproduction of these compounds can have significant cardiovascular effects, such as changes in blood vessel tone and blood vessel permeability. Chronic or acute dysfunction of the normal regulation can result in morbidity and even mortality of the subject. However, it is unclear which component, or components, of the array of eicosanoids, cytokines, associated enzymes, and other associated compounds, directly or indirectly results in morbidity or mortality of the host.

Dysfunction of the normal glucocorticoid/glucocorticoid receptor regulatory system may be due to glucocorticoid insufficiency, glucocorticoid resistance, or to the occurrence of a T cell-activating stimulus that simply overwhelms the subject's T cell mediated immune response regulatory capacity. It is known that in subjects showing glucocorticoid insufficiency, about 70% of primary or chronic adrenocortical insufficiency (Addison's disease) is due to idiopathic atrophy of the adrenal cortex. The rest is probably caused by autoimmune processes. See, e.g., The Merck Manual, 17 Ed., M. H. Beers and R. Berkow, Eds., pp. 101-105, Merck Research Laboratories, Whitehouse Station, N.J. (1999)). Subjects can show a glucocorticoid resistance for any of several reasons. One explanation is an abnormal GRα/GRβ ratio. See, e.g., Bantel, H. et al., Gastroenterology, 114(4):1178 (2000). Another is resistance developed in response to either chronic inflammatory stimuli or chronic GC treatment. Glucocorticoid resistance can also be iatrogenic, as with the withdrawal of GC administration from a subject for whom GC use has become chronic. Furthermore, there are cases where subjects having a normally functioning T cell-mediated immune response are challenged with a T cell activating stimulus, such as toxic shock, bacterial or viral sepsis, or a graft vs. host response, that is sufficiently strong that it overwhelms the GC regulatory system.

Despite the success of therapy involving the administration of glucocorticoids to modulate the immune response in cases involving T cell activation, there continue to be instances, particularly in subjects having a glucocorticoid regulation deficiency, where such therapy is insufficient. In these cases, it would be useful to provide compositions and methods that could be used to prevent and to treat the morbidity and mortality that results from such conditions. It would be particularly useful if the compositions and methods supplemented or enhanced therapies that are known.

### SUMMARY OF THE INVENTION

Briefly, therefore, the present invention is directed to a novel method of preventing or treating a T cell mediated inflammatory/autoimmune disease or disorder in a subject having a glucocorticoid regulation deficiency, where the subject is in need of such treatment, the method comprising administering to the subject an effective amount of a cyclooxygenase-2 inhibitor or prodrug thereof. In some embodiments, the cyclooxygenase-2 inhibitor can be a cyclooxygenase-2 selective inhibitor.

The present invention is also directed to a novel method of preventing or treating morbidity and mortality associated with T cell activation in a subject having a glucocorticoid regulation deficiency, the method comprising administering to the subject an effective amount of a cyclooxygenase-2 inhibitor.

The present invention is also directed to a novel method of limiting morbidity and mortality in a subject having a glucocorticoid regulation deficiency, the method comprising administering to the subject an effective amount of a cyclooxygenase-2 inhibitor prior to, during, or after the subject has undergone a T cell activating process.

The present invention is also directed to a novel method of treating a subject for a T cell mediated inflammatory/autoimmune disease or disorder, the method comprising administering an effective amount of a cyclooxygenase-2 inhibitor to a subject having a glucocorticoid regulation deficiency after the subject has undergone a T cell activation process.

The present invention is also directed to a novel method of treating a subject for a T cell mediated disease or disorder, wherein method comprises treating the subject with a cyclooxygenase-2 inhibitor in combination with a glucocorticoid.

The present invention is also directed to a novel composition for the prevention and/or treatment of T cell mediated inflammatory/autoimmune diseases and disorders in a subject having a glucocorticoid regulation deficiency, the composition comprising a combination of a cyclooxygenase-2 inhibitor and a glucocorticoid.

The present invention is also directed to a novel pharmaceutical composition for the prevention and/or treatment of T cell mediated inflammatory/autoimmune diseases and disorders in a subject having a glucocorticoid regulation deficiency, the pharmaceutical composition comprising a pharmaceutically acceptable excipient and a combination of a cyclooxygenase-2 inhibitor and a glucocorticoid.

The present invention is also directed to a novel kit for the prevention and/or treatment of T cell mediated inflammatory/autoimmune disease or disorder in a subject having a glucocorticoid regulation deficiency, the kit comprising one dosage form comprising a cyclooxygenase-2 inhibitor and a second dosage form comprising a glucocorticoid, wherein the cyclooxygenase-2 inhibitor and a glucocorticoid are present each in an amount sufficient that the kit provides an effective amount of the combination.

Among the several advantages found to be achieved by the present invention, therefore, may be noted the provision of compositions and methods that could be used to prevent and to treat the morbidity and mortality that results from T cell mediated inflammatory/autoimmune diseases and disorders, particularly in subjects having a glucocorticoid regulation deficiency. It would be particularly useful if the compositions and methods supplemented or enhanced therapies that are known.

### BRIEF DESCRIPTION OF THE DRAWINGS

FIG. 1 shows: (A) a schematic illustration of the targeted deletion of GR exon two, wherein a targeting vector was designed in which exon two was flanked by loxP sites (triangles); (B) a Western blot test for total protein that was extracted from whole thymus or CD4⁺ thymocytes purified by flow cytometry and probed for expression of GR and where blots were re-probed for expression of actin as a loading control; and (C) a bar chart showing plasma corticosterone levels in T cell glucocorticoid receptor knock out (TGRko) and control mice in the morning, evening, two or eight hours after injection of 100 μg anti-CD3ε antibody (CD3ε is the epsilon component of the T cell receptor complex); which, taken together, illustrate that the deletion of T cell glucocorticoid receptor does not alter HPA axis regulation;

FIG. 2 shows: (A) Kaplan-Meyer plots of survival (□ control, n=10; ▪ TGRko, n=4; ◯ control+dexamethasone (DEX), n=10; ● TGRko+DEX, n=7); (B) bar charts indicating plasma cytokine levels in TGRko and control mice two and eight hours after injection of anti-CD3ε antibody (100 μg)±dexamethasone administration (200 μg 1 hour before and 8 hours after anti-CD3ε antibody administration) analyzed by enzyme linked immunosorbent assay (ELISA); and (C) a quantitated phosphorimager display of a ribonuclease protection assay (RPA) analysis of splenic RNA (2 μg) from TGRko and control mice at baseline or eight hours after injection of anti-CD3e antibody ((+) denotes positive control RNA provided by the manufacturer), where expression was normalized to glyceraldehyde phosphate dehydrogenase (GAPDH), which, taken together, illustrate that T cell GR is required for prevention of lethality and downregulation of multiple cytokines after activation; and

FIG. 3 shows plots of survival vs. time for: (A) TGRko mice which had been treated with a cyclooxygenase-2 selective inhibitor (SC-236) (solid line (s), n=7), NS-398 (alternately dashed line (n)), or vehicle (dashed line (v), n=3) one hour before anti-CD3ε antibody administration, and twice a day for two days thereafter; and (B) control mice which had been treated with mifepristone+SC-236 (m/s, solid line, n=10), mifepristone (RU-486, a GR antagonist)+vehicle (m/v, dashed line, n=8), or vehicle+vehicle (v/v, mixed line, n=3), and shows that the administration of a cyclooxygenase-2 selective inhibitor protects against mortality induced by polyclonal T cell activation in GR-deficient mice; and histological examination of the cecum in mice treated with anti-CD3ε antibody (C) demonstrates marked edema, inflammation, and mucosal disruption in the TGRko mice and rescue with Cox-2 inhibition with NS-398.

### DETAILED DESCRIPTION OF THE PREFERRED EMBODIMENTS

In accordance with the present invention, it has been discovered that morbidity and mortality associated with T cell-mediated inflammatory/autoimmune diseases and disorders in subjects that have a glucocorticoid regulation deficiency, and that are in need of such treatment, can be prevented and/or treated by the administration of a cyclooxygenase-2 inhibitor to the subject. In preferred embodiments, the cyclooxygenase-2 inhibitor can be a cyclooxygenase-2 selective inhibitor.

The inventors have shown that glucocorticoid receptor function within the T cell is essential in order to maintain the survival of a subject in the setting of polyclonal T cell activation. While T cell glucocorticoid receptor deficiency results in dysregulation of several cytokines, redundant mechanisms for down-regulation of some of these molecules, interleukin-2 (IL-2) and tumor necrosis factor-alpha (TNFα), for example, have evolved. Interferon gamma (IFNγ) regulation is relatively unique in its requirement for glucocorticoid receptor inhibition, but the inventors have discovered that IFNγ immunoneutralization does not attenuate mortality.

The inventors have now found that, surprisingly, a critical role for glucocorticoid receptor action in the activated T cell is the modulation of Cox-2 expression and eicosanoid production, and that the administration of a cyclooxygenase-2 (Cox-2) inhibitor is therapeutically useful for limiting morbidity and mortality in patients with a glucocorticoid regulation deficiency during or after a T cell activating process.

Furthermore, many patients with systemic lupus erythematosis, graft versus host disease, and other chronic T cell-dependent autoimmune and inflammatory diseases requiring chronic glucocorticoid treatment experience life-threatening flares in disease severity despite continued glucocorticoid administration. Often, these glucocorticoid-resistant states are treated with extremely high dose glucocorticoid pulses that cause diabetes mellitus, osteoporosis, and a host of other side effects. It has been discovered, however, that institution of Cox-2 inhibitor therapy can limit disease symptoms in these glucocorticoid regulation-deficient circumstances, and prevent morbidity incurred with institution of high-dose glucocorticoid therapy. Thus, one feature of the invention is the administration of a Cox-2 inhibitor in the same instances as a treating physician would presently administer a glucocorticoid—either in place of, or in combination with, a glucocorticoid—with the advantageous result being that the Cox-2 inhibitor acts more quickly to limit the morbidity associated with the T cell activating stimulus—even reducing the danger of death—and also provides a complementary mechanism to provide additive or synergistic therapeutic efficacy.

As used herein, the term “morbidity” should be understood to mean the state of being not sound and healthful; induced by a diseased or abnormal condition; or diseased. By way of example, morbidity should be interpreted to include the major clinical symptoms surrounding the OKT-3 treatment syndrome: fever, headache, chills, diarrhoea, vomiting, meningismus, respiratory distress, hypotension, intestinal hypomotility, and (in mice) piloerection.

In the course of the present invention, the inventors generated T cell-specific, GR knockout mice to aid in the determination of the role of GR in lymphocyte development and regulation. It was shown that these animals die following polyclonal T cell activation, whereas normal mice uniformly survive. This mortality is associated with dysregulation of cytokine and Cox-2 synthesis, and can be very effectively blocked with Cox-2 inhibitors, but not by cytokine neutralization. These data demonstrated that Cox-2 in T cells is a critical target for glucocorticoid effects to maintain survival. Together with data in human and animal systems, these findings strongly implicate the utility of Cox-2 inhibition in settings of human glucocorticoid insufficiency (e.g., iatrogenic adrenal suppression in GC-treated patients, Addison's disease, etc.) with infection or inflammation, or in the context of glucocorticoid resistant autoimmune and inflammatory diseases as an adjunct therapy to limit morbidity (e.g., Lupus exacerbations, rejection or graft versus host disease in transplant patients).

The present invention includes a method of preventing or treating T cell mediated inflammatory diseases and disorders, and is particularly useful for treating such maladies in a subject having a glucocorticoid regulation deficiency. The method comprises administering to the subject an effective amount of a cyclooxygenase-2 inhibitor. The Cox-2 inhibitor can be administered to the subject alone, or in combination with a glucocorticoid.

The method can also be used for prophylactic purposes, such as by administering an effective amount of a cyclooxygenase-2 inhibitor, with or without glucocorticoids, to the subject prior to the subject's undergoing a T cell activating process.

The cyclooxygenase-2 inhibitor of the present invention can be any compound that inhibits the activity or production of the cyclooxygenase-2 enzyme. Included within the meaning of the terms “cyclooxygenase-2 inhibitor”, as used herein, are Cox-2 inhibiting compounds such as acetaminophin and nonsteroidal anti-inflammatory drugs (NSAIDs), which can be non-selective, or selective (such as are described below); nitric oxide (NO) NSAIDs (i.e., NSAIDs or NSAID analogs containing a nitrite and/or nitrite ester(s) which upon release can be GI-sparing); misoprostol/NSAID combinations (e.g., Arthrotec™); Cox-2 transcription inhibitors; and Cox-2 mRNA translation inhibitors. Cox-2 inhibitors can be synthetic or natural, and natural Cox-2 inhibitors can be plant-derived, animal-derived, or microbe derived.

Examples of Cox-2 inhibitors that are useful in the present invention include, without limitation, indoles, such as etodolac, indomethacin, sulindac and tolmetin; naphthylalkanones, such as nabumetone; oxicams, such as piroxicam; para-aminophenol derivatives, such as acetaminophen; propionic acids, such as fenoprofen, flurbiprofen, ibuprofen, ketoprofen, naproxen, naproxen sodium, and oxaprozin; salicylates, such as aspirin, choline magnesium trisalicylate and diflunisal; fenamates, such as meclofenamic acid and mefenamic acid; and pyrazoles, such as phenylbutazone.

In preferred embodiments, the Cox-2 inhibitor can be a cyclooxygenase-2 selective inhibitor. The terms “cyclooxygenase-2 selective inhibitor”, or “Cox-2 selective inhibitor”, which can be used interchangeably herein, embrace compounds which selectively inhibit cyclooxygenase-2 over cyclooxygenase-1, and also include pharmaceutically acceptable salts of those compounds.

In practice, the selectivity of a Cox-2 inhibitor varies depending upon the condition under which the test is performed and on the inhibitors being tested. However, for the purposes of this specification, the selectivity of a Cox-2 inhibitor can be measured as a ratio of the in vitro or in vivo IC₅₀ value for inhibition of Cox-1, divided by the IC₅₀ value for inhibition of Cox-2 (Cox-1 IC₅₀/Cox-2 IC₅₀). A Cox-2 selective inhibitor is any inhibitor for which the ratio of Cox-1 IC₅₀ to Cox-2 IC₅₀ is greater than 1. In preferred embodiments, this ratio is greater than 2, more preferably greater than 5, yet more preferably greater than 10, still more preferably greater than 50, and more preferably still greater than 100.

As used herein, the term “IC₅₀” refers to the concentration of a compound that is required to produce 50% inhibition of cyclooxygenase activity. Preferred cyclooxygenase-2 selective inhibitors of the present invention have a cyclooxygenase-2 IC₅₀ of less than about 1 μM, more preferred of less than about 0.5 μM, and even more preferred of less than about 0.2 μM.

Preferred cycloxoygenase-2 selective inhibitors have a cyclooxygenase-1 IC₅₀ of greater than about 1 μM, and more preferably of greater than 20 μm. Such preferred selectivity may indicate an ability to reduce the incidence of common NSAID-induced side effects.

Also included within the scope of the present invention are compounds that act as prodrugs of cyclooxygenase-2-selective inhibitors. As used herein in reference to Cox-2 selective inhibitors, the term “prodrug” refers to a chemical compound that can be converted into an active Cox-2 selective inhibitor by metabolic or simple chemical processes within the body of the subject. One example of a prodrug for a Cox-2 selective inhibitor is parecoxib, which is a therapeutically effective prodrug of the tricyclic cyclooxygenase-2 selective inhibitor valdecoxib. An example of a preferred Cox-2 selective inhibitor prodrug is parecoxib sodium. A class of prodrugs of Cox-2 inhibitors is described in U.S. Pat. No. 5,932,598.

The cyclooxygenase-2 selective inhibitor of the present invention can be, for example, the Cox-2 selective inhibitor meloxicam, Formula B-1 (CAS registry number 71125-38-7), or a pharmaceutically acceptable salt or prodrug thereof.

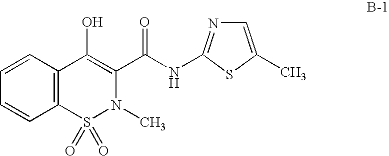

In another embodiment of the invention the cyclooxygenase-2 selective inhibitor can be the Cox-2 selective inhibitor RS 57067, 6-[[5-(4-chlorobenzoyl)-1,4-dimethyl-1H-pyrrol-2-yl]methyl]-3(2H)-pyridazinone, Formula B-2 (CAS registry number 179382-91-3), or a pharmaceutically acceptable salt or prodrug thereof.

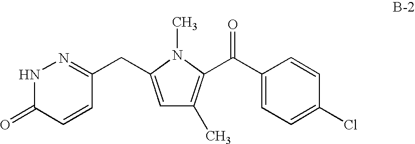

In a another embodiment of the invention the cyclooxygenase-2 selective inhibitor is of the chromene/chroman structural class that is a substituted benzopyran or a substituted benzopyran analog, and even more preferably selected from the group consisting of substituted benzothiopyrans, dihydroquinolines, or dihydronaphthalenes having the structure of any one of the compounds having a structure shown by general Formulas I, II, III, IV, V, and VI, shown below, and possessing, by way of example and not limitation, the structures disclosed in Table 1, including the diastereomers, enantiomers, racemates, tautomers, salts, esters, amides and prodrugs thereof.

Benzopyrans that can serve as a cyclooxygenase-2 selective inhibitor of the present invention include substituted benzopyran derivatives that are described in U.S. Pat. No. 6,271,253. One such class of compounds is defined by the general formula shown below in formulas I:

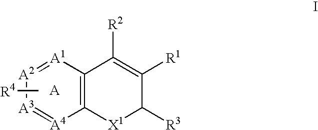

wherein X¹ is selected from O, S, CR^{c} R^{b} and NR^{a};

wherein R^{a} is selected from hydrido, C₁-C₃-alkyl, (optionally substituted phenyl)-C₁-C₃-alkyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃ -alkyl; or wherein CR^{b}R^{c} forms a 3-6 membered cycloalkyl ring;

wherein R¹ is selected from carboxyl, aminocarbonyl, C₁-C₆-alkylsulfonylaminocarbonyl and C₁-C₆-alkoxycarbonyl;

wherein R² is selected from hydrido, phenyl, thienyl, C₁-C₆-alkyl and C₂-C₆-alkenyl;

wherein R³ is selected from C₁-C₃-perfluoroalkyl, chloro, C₁-C₆-alkylthio, C₁-C₆-alkoxy, nitro, cyano and cyano-C₁-C₃-alkyl;

wherein R⁴ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-₁-C₃-hydroxyalkyl, C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the A ring atoms A¹, A², A³ and A⁴ are independently selected from carbon and nitrogen with the proviso that at least two of A¹, A², A³ and A⁴ are carbon;

or wherein R⁴ together with ring A forms a radical selected from naphthyl, quinolyl, isoquinolyl, quinolizinyl, quinoxalinyl and dibenzofuryl; or an isomer or pharmaceutically acceptable salt thereof.

Another class of benzopyran derivatives that can serve as the Cox-2 selective inhibitor of the present invention includes a compound having the structure of formula II:

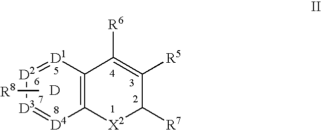

wherein X² is selected from O, S, CR^{c} R^{b} and NR^{a};

wherein R^{a} is selected from hydrido, C₁-C₃-alkyl, (optionally substituted phenyl)-C₁-C₃-alkyl, alkylsulfonyl, phenylsulfonyl, benzylsulfonyl, acyl and carboxy-C₁-C₆-alkyl;

wherein each of R^{b} and R^{c} is independently selected from hydrido, C₁-C₃-alkyl, phenyl-C₁-C₃-alkyl, C₁-C₃-perfluoroalkyl, chloro, C₁-C₆-alkylthio, C₁-C₆-alkoxy, nitro, cyano and cyano-C₁-C₃-alkyl; or wherein CR^{c} R^{b} form a cyclopropyl ring;

wherein R⁵ is selected from carboxyl, aminocarbonyl, C₁-C₆-alkylsulfonylaminocarbonyl and C₁-C₆-alkoxycarbonyl;

wherein R⁶ is selected from hydrido, phenyl, thienyl, C₂-C₆-alkynyl and C₂-C₆-alkenyl;

wherein R⁷ is selected from C₁-C₃-perfluoroalkyl, chloro, C₁-C₆-alkylthio, C₁-C₆-alkoxy, nitro, cyano and cyano-C₁-C₃-alkyl; wherein R⁸ is one or more radicals independently selected from hydrido, halo, C₁-C₆-alkyl, C₂-C₆-alkenyl, C₂-C₆-alkynyl, halo-C₂-C₆-alkynyl, aryl-C₁-C₃-alkyl, aryl-C₂-C₆-alkynyl, aryl-C₂-C₆-alkenyl, C₁-C₆-alkoxy, methylenedioxy, C₁-C₆-alkylthio, C₁-C₆-alkylsulfinyl, —O(CF₂)₂O—, aryloxy, arylthio, arylsulfinyl, heteroaryloxy, C₁-C₆-alkoxy-C₁-C₆-alkyl, aryl-C₁-C₆-alkyloxy, heteroaryl-C₁-C₆-alkyloxy, aryl-C₁-C₆-alkoxy-C₁-C₆-alkyl, C₁-C₆-haloalkyl, C₁-C₆-haloalkoxy, C₁-C₆-haloalkylthio, C₁-C₆-haloalkylsulfinyl, C₁-C₆-haloalkylsulfonyl, C₁-C₃-(haloalkyl-C₁-C₃-hydroxyalkyl), C₁-C₆-hydroxyalkyl, hydroxyimino-C₁-C₆-alkyl, C₁-C₆-alkylamino, arylamino, aryl-C₁-C₆-alkylamino, heteroarylamino, heteroaryl-C₁-C₆-alkylamino, nitro, cyano, amino, aminosulfonyl, C₁-C₆-alkylaminosulfonyl, arylaminosulfonyl, heteroarylaminosulfonyl, aryl-C₁-C₆-alkylaminosulfonyl, heteroaryl-C₁-C₆-alkylaminosulfonyl, heterocyclylsulfonyl, C₁-C₆-alkylsulfonyl, aryl-C₁-C₆-alkylsulfonyl, optionally substituted aryl, optionally substituted heteroaryl, aryl-C₁-C₆-alkylcarbonyl, heteroaryl-C₁-C₆-alkylcarbonyl, heteroarylcarbonyl, arylcarbonyl, aminocarbonyl, C₁-C₆-alkoxycarbonyl, formyl, C₁-C₆-haloalkylcarbonyl and C₁-C₆-alkylcarbonyl; and

wherein the D ring atoms D¹, D², D³ and D⁴ are independently selected from carbon and nitrogen with the proviso that at least two of D¹, D², D³ and D⁴ are carbon; or

wherein R⁸ together with ring D forms a radical selected from naphthyl, quinolyl, isoquinolyl, quinolizinyl, quinoxalinyl and dibenzofuryl; or an isomer or pharmaceutically acceptable salt thereof.

Other benzopyran Cox-2 selective inhibitors useful in the practice of the present invention are described in U.S. Pat. Nos. 6,034,256 and 6,077,850. The general formula for these compounds is shown in formula III:

Formula III is:

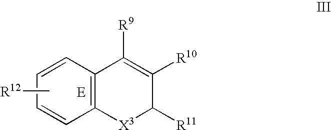

R¹² l

wherein X³ is selected from the group consisting of O or S or NR^{a};

wherein R^{a} is alkyl;

wherein R⁹ is selected from the group consisting of H and aryl;

wherein R¹⁰ is selected from the group consisting of carboxyl, aminocarbonyl, alkylsulfonylaminocarbonyl and alkoxycarbonyl;

wherein R¹¹ is selected from the group consisting of haloalkyl, alkyl, aralkyl, cycloalkyl and aryl optionally substituted with one or more radicals selected from alkylthio, nitro and alkylsulfonyl; and

wherein R¹² is selected from the group consisting of one or more radicals selected from H, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, hydroxyarylcarbonyl, nitroaryl, optionally substituted aryl, optionally substituted heteroaryl, aralkylcarbonyl, heteroarylcarbonyl, arylcarbonyl, aminocarbonyl, and alkylcarbonyl; or

wherein R¹² together with ring E forms a naphthyl radical; or an isomer or pharmaceutically acceptable salt thereof; and including the diastereomers, enantiomers, racemates, tautomers, salts, esters, amides and prodrugs thereof.

A related class of compounds useful as cyclooxygenase-2 selective inhibitors in the present invention is described by Formulas IV and V:

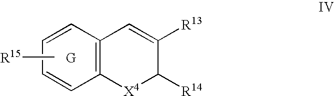

wherein X⁴ is selected from O or S or NR^{a};

wherein R^{a} is alkyl;

wherein R¹³ is selected from carboxyl, aminocarbonyl, alkylsulfonylaminocarbonyl and alkoxycarbonyl;

wherein R¹⁴ is selected from haloalkyl, alkyl, aralkyl, cycloalkyl and aryl optionally substituted with one or more radicals selected from alkylthio, nitro and alkylsulfonyl; and

wherein R¹⁵ is one or more radicals selected from hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl;

or wherein R¹⁵ together with ring G forms a naphthyl radical; or an isomer or pharmaceutically acceptable salt thereof.

Formula V is:

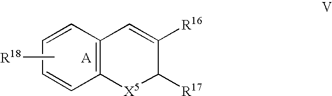

wherein:

X⁵ is selected from the group consisting of O or S or NR^{b};

R^{b} is alkyl;

R¹⁶ is selected from the group consisting of carboxyl, aminocarbonyl, alkylsulfonylaminocarbonyl and alkoxycarbonyl;

R¹⁷ is selected from the group consisting of haloalkyl, alkyl, aralkyl, cycloalkyl and aryl, wherein haloalkyl, alkyl, aralkyl, cycloalkyl, and aryl each is independently optionally substituted with one or more radicals selected from the group consisting of alkylthio, nitro and alkylsulfonyl; and

R¹⁸ is one or more radicals selected from the group consisting of hydrido, halo, alkyl, aralkyl, alkoxy, aryloxy, heteroaryloxy, aralkyloxy, heteroaralkyloxy, haloalkyl, haloalkoxy, alkylamino, arylamino, aralkylamino, heteroarylamino, heteroarylalkylamino, nitro, amino, aminosulfonyl, alkylaminosulfonyl, arylaminosulfonyl, heteroarylaminosulfonyl, aralkylaminosulfonyl, heteroaralkylaminosulfonyl, heterocyclosulfonyl, alkylsulfonyl, optionally substituted aryl, optionally substituted heteroaryl, aralkylcarbonyl, heteroarylcarbonyl, arylcarbonyl, aminocarbonyl, and alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of lower haloalkyl, lower cycloalkyl and phenyl; and

R¹⁸ is one or more radicals selected from the group of consisting of hydrido, halo, lower alkyl, lower alkoxy, lower haloalkyl, lower haloalkoxy, lower alkylamino, nitro, amino, aminosulfonyl, lower alkylaminosulfonyl, 5-membered heteroarylalkylaminosulfonyl, 6-membered heteroarylalkylaminosulfonyl, lower aralkylaminosulfonyl, 5-membered nitrogen-containing heterocyclosulfonyl, 6-membered-nitrogen containing heterocyclosulfonyl, lower alkylsulfonyl, optionally substituted phenyl, lower aralkylcarbonyl, and lower alkylcarbonyl; or

wherein R¹⁸ together with ring A forms a naphthyl radical; or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is carboxyl;

R¹⁷ is lower haloalkyl; and

R¹⁸ is one or more radicals selected from the group consisting of hydrido, halo, lower alkyl, lower haloalkyl, lower haloalkoxy, lower alkylamino, amino, aminosulfonyl, lower alkylaminosulfonyl, 5-membered heteroarylalkylaminosulfonyl, 6-membered heteroarylalkylaminosulfonyl, lower aralkylaminosulfonyl, lower alkylsulfonyl, 6-membered nitrogen-containing heterocyclosulfonyl, optionally substituted phenyl, lower aralkylcarbonyl, and lower alkylcarbonyl; or wherein R¹⁸ together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting of fluoromethyl, chloromethyl, dichloromethyl, trichloromethyl, pentafluoroethyl, heptafluoropropyl, difluoroethyl, difluoropropyl, dichloroethyl, dichloropropyl, difluoromethyl, and trifluoromethyl; and

R¹⁸ is one or more radicals selected from the group consisting of hydrido, chloro, fluoro, bromo, iodo, methyl, ethyl, isopropyl, tert-butyl, butyl, isobutyl, pentyl, hexyl, methoxy, ethoxy, isopropyloxy, tertbutyloxy, trifluoromethyl, difluoromethyl, trifluoromethoxy, amino, N,N-dimethylamino, N,N-diethylamino, N-phenylmethylaminosulfonyl, N-phenylethylaminosulfonyl, N-(2-furylmethyl)aminosulfonyl, nitro, N,N-dimethylaminosulfonyl, aminosulfonyl, N-methylaminosulfonyl, N-ethylsulfonyl, 2,2-dimethylethylaminosulfonyl, N,N-dimethylaminosulfonyl, N-(2-methylpropyl)aminosulfonyl, N-morpholinosulfonyl, methylsulfonyl, benzylcarbonyl, 2,2-dimethylpropylcarbonyl, phenylacetyl and phenyl; or

wherein R² together with ring A forms a naphthyl radical;

or an isomer or pharmaceutically acceptable salt thereof.

The cyclooxygenase-2 selective inhibitor may also be a compound of Formula V, wherein:

X⁵ is selected from the group consisting of oxygen and sulfur;

R¹⁶ is selected from the group consisting of carboxyl, lower alkyl, lower aralkyl and lower alkoxycarbonyl;

R¹⁷ is selected from the group consisting trifluoromethyl and pentafluoroethyl; and

R¹⁸ is one or more radicals selected from the group consisting of hydrido, chloro, fluoro, bromo, iodo, methyl, ethyl, isopropyl, tert-butyl, methoxy, trifluoromethyl, trifluoromethoxy, N-phenylmethylaminosulfonyl, N-phenylethylaminosulfonyl, N-(2-furylmethyl)aminosulfonyl, N,N-dimethylaminosulfonyl, N-methylaminosulfonyl, N-(2,2-dimethylethyl)aminosulfonyl, dimethylaminosulfonyl, 2-methylpropylaminosulfonyl, N-morpholinosulfonyl, methylsulfonyl, benzylcarbonyl, and phenyl; or wherein R¹⁸ together with ring A forms a naphthyl radical;

or an isomer or prodrug thereof.

The cyclooxygenase-2 selective inhibitor of the present invention can also be a compound having the structure of Formula VI:

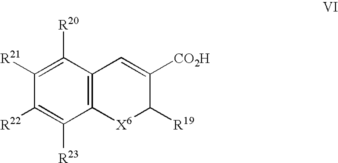

wherein:

X⁶ is selected from the group consisting of O and S;

R¹⁹ is lower haloalkyl;

R²⁰ is selected from the group consisting of hydrido, and halo;

R²¹ is selected from the group consisting of hydrido, halo, lower alkyl, lower haloalkoxy, lower alkoxy, lower aralkylcarbonyl, lower dialkylaminosulfonyl, lower alkylaminosulfonyl, lower aralkylaminosulfonyl, lower heteroaralkylaminosulfonyl, 5-membered nitrogen-containing heterocyclosulfonyl, and 6- membered nitrogen-containing heterocyclosulfonyl;

R²² is selected from the group consisting of hydrido, lower alkyl, halo, lower alkoxy, and aryl; and

R²³ is selected from the group consisting of the group consisting of hydrido, halo, lower alkyl, lower alkoxy, and aryl;

or an isomer or prodrug thereof.

The cyclooxygenase-2 selective inhibitor can also be a compound of having the structure of Formula VI, wherein:

X⁶ is selected from the group consisting of O and S;

R¹⁹ is selected from the group consisting of trifluoromethyl and pentafluoroethyl;

R²⁰ is selected from the group consisting of hydrido, chloro, and fluoro;

R²¹ is selected from the group consisting of hydrido, chloro, bromo, fluoro, iodo, methyl, tert-butyl, trifluoromethoxy, methoxy, benzylcarbonyl, dimethylaminosulfonyl, isopropylaminosulfonyl, methylaminosulfonyl, benzylaminosulfonyl, phenylethylaminosulfonyl, methylpropylaminosulfonyl, methylsulfonyl, and morpholinosulfonyl;

R²² is selected from the group consisting of hydrido, methyl, ethyl, isopropyl, tert-butyl, chloro, methoxy, diethylamino, and phenyl; and

R²³ is selected from the group consisting of hydrido, chloro, bromo, fluoro, methyl, ethyl, tert-butyl, methoxy, and phenyl;

or an isomer or prodrug thereof.

TABLE 1
Examples of Chromene Cox-2 Selective inhibitors

| Compound | |
|---|---|
| Number | Structural Formula |
| B-3 | 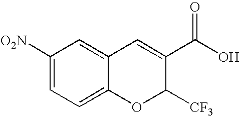 |
| | 6-Nitro-2-trifluoromethyl-2H-1- |
| | benzopyran-3-carboxylic acid |
| B-4 | 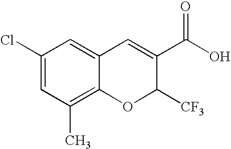 |
| | 6-Chloro-8-methyl-2-trifluoromethyl- |
| | 2H-1-benzopyran-3-carboxylic acid |
| B-5 | 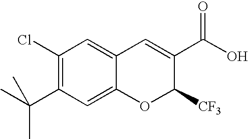 |
| | ((S)-6-Chloro-7-(1,1-dimethylethyl)-2-(trifluoro- |
| | methyl-2H-1-benzopyran-3-carboxylic acid |
| B-6 | 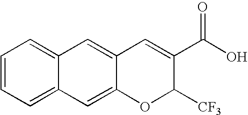 |
| | 2-Trifluoromethyl-2H-naphtho[2,3-b] |
| | pyran-3-carboxylic acid |
| B-7 | 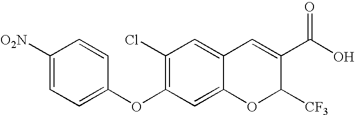 |
| | 6-Chloro-7-(4-nitrophenoxy)-2-(trifluoromethyl)-2H-1- |
| | benzopyran-3-carboxylic acid |
| B-8 | 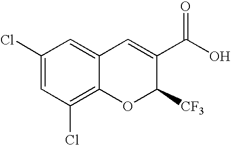 |
| | ((S)-6,8-Dichloro-2-(trifluoromethyl)- |
| | 2H-1-benzopyran-3-carboxylic acid |
| B-9 | 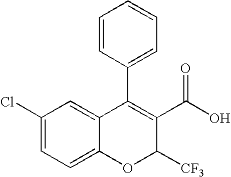 |
| | 6-Chloro-2-(trifluoromethyl)-4-phenyl-2H- |
| | 1-benzopyran-3-carboxylic acid |
| B-10 | 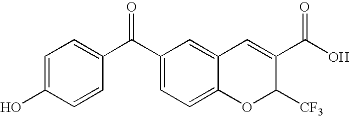 |
| | 6-(4-Hydroxybenzoyl)-2-(trifluoromethyl)- |
| | 2H-1-benzopyran-3-carboxylic acid |
| B-11 | 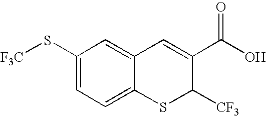 |
| | 2-(Trifluoromethyl)-6-[(trifluoromethyl)thio]- |
| | 2H-1-benzothiopyran-3-carboxylic acid |
| B-12 | 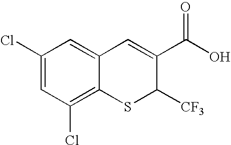 |
| | 6,8-Dichloro-2-trifluoromethyl-2H-1- |
| | benzothiopyran-3-carboxylic acid |
| B-13 | 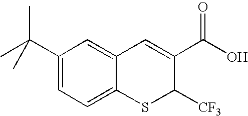 |
| | 6-(1,1-Dimethylethyl)-2-(trifluoromethyl)- |
| | 2H-1-benzothiopyran-3-carboxylic acid |
| B-14 | 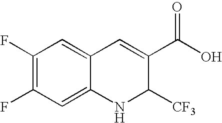 |
| | 6,7-Difluoro-1,2-dihydro-2-(trifluoro |
| | methyl)-3-quinolinecarboxylic acid |
| B-15 | 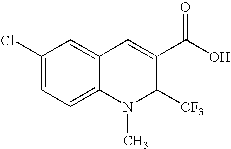 |
| | 6-Chloro-1,2-dihydro-1-methyl-2-(trifluoro |
| | methyl)-3-quinolinecarboxylic acid |
| B-16 | 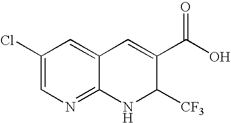 |
| | 6-Chloro-2-(trifluoromethyl)-1,2-dihydro |
| | [1,8]naphthyridine-3-carboxylic acid |
| B-17 | 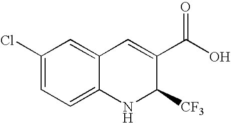 |
| | ((S)-6-Chloro-1,2-dihydro-2-(trifluoro |
| | methyl)-3-quinolinecarboxylic acid |

Examples of specific compounds that are useful for the cyclooxygenase-2 selective inhibitor include (without limitation):

a1) 8-acetyl-3-(4-fluorophenyl)-2-(4-methylsulfonyl)phenyl-imidazo(1,2-a)pyridine;

a2) 5,5-dimethyl-4-(4-methylsulfonyl)phenyl-3-phenyl-2-(5H)-furanone;

a3) 5-(4-fluorophenyl)-1-[4-(methylsulfonyl)phenyl]-3-(trifiuoromethyl)pyrazole;

a4) 4-(4-fluorophenyl)-5-[4-(methylsulfonyl)phenyl]-1-phenyl-3-(trifluoromethyl)pyrazole;

a5) 4-(5-(4-chlorophenyl)-3-(4-methoxyphenyl)-1H-pyrazol-1-yl)benzenesulfonamide

a6) 4-(3,5-bis(4-methylphenyl)-1H-pyrazol-1-yl)benzenesulfonamide;

a7) 4-(5-(4-chlorophenyl)-3-phenyl-1H-pyrazol-1-yl)benzenesulfonamide;

a8) 4-(3,5-bis(4-methoxyphenyl)-1H-pyrazol-1-yl)benzenesulfonamide;

a9) 4-(5-(4-chlorophenyl)-3-(4-methylphenyl)-1H-pyrazol-1-yl)benzenesulfonamide;

a10) 4-(5-(4-chlorophenyl)-3-(4-nitrophenyl)-1H-pyrazol-1-yl)benzenesulfonamide;

b1) 4-(5-(4-chlorophenyl)-3-(5-chloro-2-thienyl)-1H-pyrazol-1-yl)benzenesulfonamide;

b2) 4-(4-chloro-3,5-diphenyl-1H-pyrazol-1-yl)benzenesulfonamide

b3) 4-[5-(4-chlorophenyl)-3-(trifluoromethyl)-1H-pyrazol-1-yl]benzenesulfonamide;

b4) 4-[5-phenyl-3-(trifluoromethyl)-1H-pyrazol-1-yl]benzenesulfonamide;

b5) 4-[5-(4-fluorophenyl)-3-(trifluoromethyl)-1H-pyrazol-1-yl]benzenesulfonamide;

b6) 4-[5-(4-methoxyphenyl)-3-(trifluoromethyl)-1H-pyrazol-1-yl]benzenesulfonamide;

b7) 4-[5-(4-chlorophenyl)-3-(difluoromethyl)-1H-pyrazol-1-yl]benzenesulfonamide;

b8) 4-[5-(4-methylphenyl)-3-(trifluoromethyl)-1H-pyrazol-1-yl]benzenesulfonamide;

b9) 4-[4-chloro-5-(4-chlorophenyl)-3-(trifluoromethyl)-1H-pyrazol-1-yl]benzenesulfonamide;

b10) 4-[3-(difluoromethyl)-5-(4-methylphenyl)-1H-pyrazol-1-yl]benzenesulfonamide;

c1) 4-[3-(difluoromethyl)-5-phenyl-1H-pyrazol-1-yl]benzenesulfonamide;

c2) 4-[3-difluoromethyl)-5-(4-methoxyphenyl)-1H-pyrazol-1-yl]benzenesulfonamide;

c3) 4-[3-cyano-5-(4-fluorophenyl)-1H-pyrazol-1-yl]benzenesulfonamide;

c4) 4-[3-(difluoromethyl)-5-(3-fluoro-4-methoxyphenyl)-1H-pyrazol-1-yl]benzenesulfonamide;

c5) 4-[5-(3-fluoro-4-methoxyphenyl)-3-(trifluoromethyl)-1H-pyrazol-1-yl]benzenesulfonamide;

c6) 4-[4-chloro-5-phenyl-1H-pyrazol-1-yl]benzenesulfonamide;

c7) 4-[5-(4-chlorophenyl)-3-(hydroxymethyl)-1H-pyrazol-1-yl]benzenesulfonamide;

c8) 4-[5-(4-(N,N-dimethylamino)phenyl)-3-(trifluoromethyl)-1H-pyrazol-1-yl]benzenesulfonamide;

c9) 5-(4-fluorophenyl)-6-[4-(methylsulfonyl)phenyl]spiro[2.4]hept-5-ene;

c10) 4-[6-(4-fluorophenyl)spiro[2.4]hept-5-en-5-yl]benzenesulfonamide;

d1) 6-(4-fluorophenyl)-7-[4-(methylsulfonyl)phenyl]spiro[3.4]oct-6-ene;

d2) 5-(3-chloro-4-methoxyphenyl)-6-[4-(methylsulfonyl)phenyl]spiro[2.4]hept-5-ene;

d3) 4-[6-(3-chloro-4-methoxyphenyl)spiro[2.4]hept-5-en-5-yl]benzenesulfonamide;

d4) 5-(3,5-dichloro-4-methoxyphenyl)-6-[4-(methylsulfonyl)phenyl]spiro[2.4]hept-5-ene;

d5) 5-(3-chloro-4-fluorophenyl)-6-[4-(methylsulfonyl)phenyl]spiro[2.4]hept-5-ene;

d6) 4-[6-(3,4-dichlorophenyl)spiro[2.4]hept-5-en-5-yl]benzenesulfonamide;

d7) 2-(3-chloro-4-fluorophenyl)-4-(4-fluorophenyl)-5-(4-methylsulfonylphenyl)thiazole;

d8) 2-(2-chlorophenyl)-4-(4-fluorophenyl)-5-(4-methylsulfonylphenyl)thiazole;

d9) 5-(4-fluorophenyl)-4-(4-methylsulfonylphenyl)-2-methylthiazole;

d10) 4-(4-fluorophenyl)-5-(4-methylsulfonylphenyl)-2-trifluoromethylthiazole;

e1) 4-(4-fluorophenyl)-5-(4-methylsulfonylphenyl)-2-(2-thienyl)thiazole;

e2) 4-(4-fluorophenyl)-5-(4-methylsulfonylphenyl)-2-benzylaminothiazole;

e3) 4-(4-fluorophenyl)-5-(4-methylsulfonylphenyl)-2-(1-propylamino)thiazole;

e4) 2-[(3,5-dichlorophenoxy)methyl)-4-(4-fluorophenyl)-5-[4-(methylsulfonyl)phenyl]thiazole;

e5) 5-(4-fluorophenyl)-4-(4-methylsulfonylphenyl)-2-trifluoromethylthiazole;

e6) 1-methylsulfonyl-4-[1,1-dimethyl-4-(4-fluorophenyl)cyclopenta-2,4-dien-3-yl]benzene;

e7) 4-[4-(4-fluorophenyl)-1,1-dimethylcyclopenta-2,4-dien-3-yl]benzenesulfonamide;

e8) 5-(4-fluorophenyl)-6-[4-(methylsulfonyl)phenyl]spiro[2.4]hepta-4,6-diene;

e9) 4-[6-(4-fluorophenyl)spiro[2.4]hepta-4,6-dien-5-yl]benzenesulfonamide;

e10) 6-(4-fluorophenyl)-2-methoxy-5-[4-(methylsulfonyl)phenyl]-pyridine-3-carbonitrile;

f1) 2-bromo-6-(4-fluorophenyl)-5-[4-(methylsulfonyl)phenyl]-pyridine-3-carbonitrile;

f2) 6-(4-fluorophenyl)-5-[4-(methylsulfonyl)phenyl]-2-phenyl-pyridine-3-carbonitrile;

f3) 4-[2-(4-methylpyridin-2-yl)-4-(trifluoromethyl)-1H-imidazol-1-yl]benzenesulfonamide;

f4) 4-[2-(5-methylpyridin-3-yl)-4-(trifluoromethyl)-1H-imidazol-1-yl]benzenesulfonamide;

f5) 4-[2-(2-methylpyridin-3-yl)-4-(trifluoromethyl)-1H-imidazol-1-yl]benzenesulfonamide;

f6) 3-[1-[4-(methylsulfonyl)phenyl]-4-(trifluoromethyl)-1H-imidazol-2-yl]pyridine;

f7) 2-[1-[4-(methylsulfonyl)phenyl-4-(trifluoromethyl)-1H-imidazol-2-yl]pyridine;

f8) 2-methyl-4-[1-[4-(methylsulfonyl)phenyl-4-(trifluoromethyl)-1H-imidazol-2-yl]pyridine;

f9) 2-methyl-6-[1-[4-(methylsulfonyl)phenyl-4-(trifluoromethyl)-1H-imidazol-2-yl]pyridine;

f10) 4-[2-(6-methylpyridin-3-yl)-4-(trifluoromethyl)-1H-imidazol-1-yl]benzenesulfonamide;

g1) 2-(3,4-difluorophenyl)-1-[4-(methylsulfonyl)phenyl]-4-(trifluoromethyl)-1H-imidazole;

g2) 4-[2-(4-methylphenyl)-4-(trifluoromethyl)-1H-imidazol-1-yl]benzenesulfonamide;

g3) 2-(4-chlorophenyl)-1-[4-(methylsulfonyl)phenyl]-4-methyl-1H-imidazole;

g4) 2-(4-chlorophenyl)-1-[4-(methylsulfonyl)phenyl]-4-phenyl-1H-imidazole;

g5) 2-(4-chlorophenyl)-4-(4-fluorophenyl)-1-[4-(methylsulfonyl)phenyl]-1H-imidazole;

g6) 2-(3-fluoro-4-methoxyphenyl)-1-[4-(methylsulfonyl)phenyl-4-(trifluoromethyl)-1H-imidazole;

g7) 1-[4-(methylsulfonyl)phenyl]-2-phenyl-4-trifluoromethyl-1H-imidazole;

g8) 2-(4-methylphenyl)-1-[4-(methylsulfonyl)phenyl]-4-trifluoromethyl-1H-imidazole;

g9) 4-[2-(3-chloro-4-methylphenyl)-4-(trifluoromethyl)-1H-imidazol-1-yl]benzenesulfonamide;

g10) 2-(3-fluoro-5-methylphenyl)-1-[4-(methylsulfonyl)phenyl]-4-(trifluoromethyl)-1H-imidazole;

h1) 4-[2-(3-fluoro-5-methylphenyl)-4-(trifluoromethyl)-1H-imidazol-1-yl]benzenesulfonamide;

h2) 2-(3-methylphenyl)-1-[4-(methylsulfonyl)phenyl]-4-trifluoromethyl-1H-imidazole;

h3) 4-[2-(3-methylphenyl)-4-trifluoromethyl-1H-imidazol-1-yl]benzenesulfonamide;

h4) 1-[4-(methylsulfonyl) phenyl]-2-(3-chlorophenyl)-4-trifluoromethyl-1H-imidazole;

h5) 4-[2-(3-chlorophenyl)-4-trifluoromethyl-1H-imidazol-1-yl]benzenesulfonamide;

h6) 4-[2-phenyl-4-trifluoromethyl-1H-imidazol-1-yl]benzenesulfonamide;

h7) 4-[2-(4-methoxy-3-chlorophenyl)-4-trifluoromethyl-1H-imidazol-1-yl]benzenesulfonamide;

h8) 1-allyl-4-(4-fluorophenyl)-3-[4-(methylsulfonyl)phenyl]-5-(trifluoromethyl)-1H-pyrazole;

h10) 4-[1-ethyl-4-(4-fluorophenyl)-5-(trifluoromethyl)-1H-pyrazol-3-yl]benzenesulfonamide;

i1) N-phenyl-[4-(4-luorophenyl)-3-[4-(methylsulfonyl)phenyl]-5-(trifluoromethyl)-1H-pyrazol-1-yl]acetamide;

i2) ethyl [4-(4-fluorophenyl)-3-[4-(methylsulfonyl)phenyl]-5-(trifluoromethyl)-1H-pyrazol-1-yl]acetate;

i3) 4-(4-fluorophenyl)-3-[4-(methylsulfonyl)phenyl]-1-(2-phenylethyl)-1H-pyrazole;

i4) 4-(4-fluorophenyl)-3-[4-(methylsulfonyl)phenyl]-1-(2-phenylethyl)-5-(trifluoromethyl)pyrazole;

i5) 1-ethyl-4-(4-fluorophenyl)-3-[4-(methylsulfonyl)phenyl]-5-(trifluoromethyl)-1H-pyrazole;

i6) 5-(4-fluorophenyl)-4-(4-methylsulfonylphenyl)-2-trifluoromethyl-1H-imidazole;

i7) 4-[4-(methylsulfonyl)phenyl]-5-(2-thiophenyl)-2-(trifluoromethyl)-1H-imidazole;

i8) 5-(4-fluorophenyl)-2-methoxy-4-[4-(methylsulfonyl)phenyl]-6-(trifluoromethyl)pyridine;

i9) 2-ethoxy-5-(4-fluorophenyl)-4-[4-(methylsulfonyl)phenyl]-6-(trifiuoromethyl)pyridine;

i10) 5-(4-fluorophenyl)-4-[4-(methylsulfonyl)phenyl]-2-(2-propynyloxy)-6-(trifluoromethyl)pyridine;

j1) 2-bromo-5-(4-fluorophenyl)-4-[4-(methylsulfonyl)phenyl]-6-(trifluoromethyl)pyridine;

j2) 4-[2-(3-chloro-4-methoxyphenyl)-4,5-difluorophenyl]benzenesulfonamide;

j3) 1-(4-fluorophenyl)-2-[4-(methylsulfonyl)phenyl]benzene;

j4) 5-difluoromethyl-4-(4-methylsulfonylphenyl)-3-phenylisoxazole;

j5) 4-[3-ethyl-5-phenylisoxazol-4-yl]benzenesulfonamide;

j6) 4-[5-difluoromethyl-3-phenylisoxazol-4-yl]benzenesulfonamide;

j7) 4-[5-hydroxymethyl-3-phenylisoxazol-4-yl]benzenesulfonamide;

j8) 4-[5-methyl-3-phenyl-isoxazol-4-yl]benzenesulfonamide;

j9) 1-[2-(4-fluorophenyl)cyclopenten-1-yl]-4-(methylsulfonyl)benzene;

j10) 1-[2-(4-fluoro-2-methylphenyl)cyclopenten-1-yl]-4-(methylsulfonyl)benzene;

k1) 1-[2-(4-chlorophenyl)cyclopenten-1-yl]-4-(methylsulfonyl)benzene;

k2) 1-[2-(2,4-dichlorophenyl)cyclopenten-1-yl]-4-(methylsulfonyl)benzene;

k3) 1-[2-(4-trifluoromethylphenyl)cyclopenten-1-yl]-4-(methylsulfonyl)benzene;

k4) 1-[2-(4-methylthiophenyl)cyclopenten-1-yl]-4-(methylsulfonyl)benzene;

k5) 1-[2-(4-fluorophenyl)-4,4-dimethylcyclopenten-1-yl]-4-(methylsulfonyl)benzene;

k6) 4-[2-(4-fluorophenyl)-4,4-dimethylcyclopenten-1-yl]benzenesulfonamide;

k7) 1-[2-(4-chlorophenyl)-4,4-dimethylcyclopenten-1-yl]-4-(methylsulfonyl)benzene;

k8) 4-[2-(4-chlorophenyl)-4,4-dimethylcyclopenten-1-yl]benzenesulfonamide;

k9) 4-[2-(4-fluorophenyl)cyclopenten-1-yl]benzenesulfonamide;

k10) 4-[2-(4-chlorophenyl)cyclopenten-1-yl]benzenesulfonamide;

l1) 1-[2-(4-methoxyphenyl)cyclopenten-1-yl]-4-(methylsulfonyl)benzene;

l2) 1-[2-(2,3-difluorophenyl)cyclopenten-1-yl]-4-(methylsulfonyl)benzene;

l3) 4-[2-(3-fluoro-4-methoxyphenyl)cyclopenten-1-yl]benzenesulfonamide;

l4) 1-[2-(3-chloro-4-methoxyphenyl)cyclopenten-1-yl]-4-(methylsulfonyl)benzene;

l5) 4-[2-(3-chloro-4-fluorophenyl)cyclopenten-1-yl]benzenesulfonamide;

l6) 4-[2-(2-methylpyridin-5-yl)cyclopenten-1-yl]benzenesulfonamide;

l7) ethyl 2-[4-(4-fluorophenyl)-5-[4-(methylsulfonyl) phenyl]oxazol-2-yl]-2-benzyl-acetate;

l8) 2-[4-(4-fluorophenyl)-5-[4-(methylsulfonyl)phenyl]oxazol-2-yl]acetic acid;

l9) 2-( tert-butyl)-4-(4-fluorophenyl)-5-[4-(methylsulfonyl)phenyl]oxazole;

l10) 4-(4-fluorophenyl)-5-[4-(methylsulfonyl)phenyl]-2-phenyloxazole;

m1) 4-(4-fluorophenyl)-2-methyl-5-[4-(methylsulfonyl)phenyl]oxazole; and

m2) 4-[5-(3-fluoro-4-methoxyphenyl)-2-trifluoromethyl-4-oxazolyl]benzenesulfonamide.

m3) 6-chloro-2-trifluoromethyl-2H-1-benzopyran-3-carboxylic acid;

m4) 6-chloro-7-methyl-2-trifluoromethyl-2H-1-benzopyran-3-carboxylic acid;

m5) 8-(1-methylethyl)-2-trifluoromethyl-2H-1-benzopyran-3-carboxylic acid;

m6) 6-chloro-7-( 1,1-dimethylethyl)-2-trifluoromethyl-2H-1-benzopyran-3-carboxylic acid;

m7) 6-chloro-8-( 1-methylethyl)-2-trifluoromethyl-2H-1-benzopyran-3-carboxylic acid;

m8) 2-trifluoromethyl-3H-naphthopyran-3-carboxylic acid

m9) 7-(1,1-dimethylethyl)-2-trifluoromethyl-2H-1-benzopyran-3-carboxylic acid;

m10) 6-bromo-2-trifluoromethyl-2H-1-benzopyran-3-carboxylic acid;

n1) 8-chloro-2-trifluoromethyl-2H-1-benzopyran-3-carboxylic acid;

n2) 6-trifluoromethoxy-2-trifluoromethyl-2H-1-benzopyran-3-carboxylic acid;

n3) 5,7-dichloro-2-trifluoromethyl-2H-1-benzopyran-3-carboxylic acid;

n4) 8-phenyl-2-trifluoromethyl-2H-1-benzopyran-3-carboxylic acid;

n5) 7,8-dimethyl-2-trifluoromethyl-2H-1-benzopyran-3-carboxylic acid;

n6) 6,8-bis(dimethylethyl)-2-trifluoromethyl-2H-1-benzopyran-3-carboxylic acid;

n7) 7-(1-methylethyl)-2-trifluoromethyl-2H-1-benzopyran-3-carboxylic acid;

n8) 7-phenyl-2-trifluoromethyl-2H-1-benzopyran-3-carboxylic acid;

n9) 6-chloro-7-ethyl-2-trifluoromethyl-2H-1-benzopyran-3-carboxylic acid;

n10) 6-chloro-8-ethyl-2-trifluoromethyl-2H-1-benzopyran-3-carboxylic acid;

o1) 6-chloro-7-phenyl-2-trifluoromethyl-2H-1-benzopyran-3-carboxylic acid;

o2) 6,7-dichloro-2-trifluoromethyl-2H-1-benzopyran-3-carboxylic acid;

o3) 6,8-dichloro-2-trifluoromethyl-2H-1-benzopyran-3-carboxylic acid;

o4) 2-trifluoromethyl-3H-naptho[2,1-b]pyran-3-carboxylic acid;

o5) 6-chloro-8-methyl-2-trifluoromethyl-2H-1-benzopyran-3-carboxylic acid;

o6) 8-chloro-6-methyl-2-trifluoromethyl-2H-1-benzopyran-3-carboxylic acid;

o7) 8-chloro-6-methoxy-2-trifluoromethyl-2H-1-benzopyran-3-carboxylic acid;

o8) 6-bromo-8-chloro-2-trifluoromethyl-2H-1-benzopyran-3-carboxylic acid;

o9) 8-bromo-6-fluoro-2-trifluoromethyl-2H-1-benzopyran-3-carboxylic acid;

o10) 8-bromo-6-methyl-2-trifluoromethyl-2H-1-benzopyran-3-carboxylic acid;

p1) 8-bromo-5-fluoro-2-trifluoromethyl-2H-1-benzopyran-3-carboxylic acid;

p2) 6-chloro-8-fluoro-2-trifluoromethyl-2H-1-benzopyran-3-carboxylic acid;

p3) 6-bromo-8-methoxy-2-trifluoromethyl-2H-1-benzopyran-3-carboxylic acid;

p4) 6-[[(phenylmethyl)amino]sulfonyl]-2-trifluoromethyl-2H-1-benzopyran-3-carboxylic acid;

p5) 6-[(dimethylamino)sulfonyl]-2-trifluoromethyl-2H-1-benzopyran-3-carboxylic acid;

p6) 6-[(methylamino)sulfonyl]-2-trifluoromethyl-2H-1-benzopyran-3-carboxylic acid;

p7) 6-[(4-morpholino)sulfonyl]-2-trifluoromethyl-2H-1-benzopyran-3-carboxylic acid;

p8) 6-[(1,1-dimethylethyl)aminosulfonyl]-2-trifluoromethyl-2H-1-benzopyran-3-carboxylic acid;

p9) 6-[(2-methylpropyl)aminosulfonyl]-2-trifluoromethyl-2H-1-benzopyran-3-carboxylic acid;

p10) 6-methylsulfonyl-2-trifluoromethyl-2H-1-benzopyran-3-carboxylic acid;

q1) 8-chloro-6-[[(phenylmethyl)amino]sulfonyl]-2-trifluoromethyl-2H-1-benzopyran-3-carboxylic acid;

q2) 6-phenylacetyl-2-trifluoromethyl-2H-1-benzopyran-3-carboxylic acid;

q3) 6,8-dibromo-2-trifluoromethyl-2H-1-benzopyran-3-carboxylic acid;

q4) 8-chloro-5,6-dimethyl-2-trifluoromethyl-2H-1-benzopyran-3-carboxylic acid;

q5) 6,8-dichloro-(S)-2-trifluoromethyl-2H-1-benzopyran-3-carboxylic acid;

q6) 6-benzylsulfonyl-2-trifluoromethyl-2H-1-benzopyran-3-carboxylic acid;

q7) 6-[[N-(2-furylmethyl)amino]sulfonyl]-2-trifluoromethyl-2H-1-benzopyran-3-carboxylic acid;

q8) 6-[[N-(2-phenylethyl)amino]sulfonyl]-2-trifluoromethyl-2H-1-benzopyran-3-carboxylic acid;

q9) 6-iodo-2-trifluoromethyl-2H-1-benzopyran-3-carboxylic acid;

q10) 7-(1,1-dimethylethyl)-2-pentafluoroethyl-2H-1-benzopyran-3-carboxylic acid;

r1) 5,5-dimethyl-3-(3-fluorophenyl)-4-(4-methyl-sulphonyl-2(5H)-fluranone;

r2) 6-chloro-2-trifluoromethyl-2H-1-benzothiopyran-3-carboxylic acid;

r3) 4-[5-(4-chlorophenyl)-3-(trifluoromethyl)-1H-pyrazol-1-yl]benzenesulfonamide;

r4) 4-[5-(4-methylphenyl)-3-(trifluoromethyl)-1H-pyrazol-1-yl]benzenesulfonamide;

r5) 4-[5-(3-fluoro-4-methoxyphenyl)-3-(difluoromethyl)-1H-pyrazol-1-yl]benzenesulfonamide;

r6) 3-[1-[4-(methylsulfonyl)phenyl]-4-trifluoromethyl-1H-imidazol-2-yl]pyridine;

r7) 2-methyl-5-[1-[4-(methylsulfonyl)phenyl]-4-trifluoromethyl-1H-imidazol-2-yl]pyridine;

r8) 4-[2-(5-methylpyridin-3-yl)-4-(trifluoromethyl)-1H-imidazol-1-yl]benzenesulfonamide;

r9) 4-[5-methyl-3-phenylisoxazol-4-yl]benzenesulfonamide;

r10) 4-[5-hydroxymethyl-3-phenylisoxazol-4-yl]benzenesulfonamide;

s1) [2-trifluoromethyl-5-(3,4-difluorophenyl)-4-oxazolyl]benzenesulfonamide;

s2) 4-[2-methyl-4-phenyl-5-oxazolyl]benzenesulfonamide; or

s3) 4-[5-(3-fluoro-4-methoxyphenyl-2-trifluoromethyl)-4-oxazolyl]benzenesulfonamide;

or a pharmaceutically acceptable salt or prodrug thereof.

In a further preferred embodiment of the invention the cyclooxygenase inhibitor can be selected from the class of tricyclic cyclooxygenase-2 selective inhibitors represented by the general structure of formula VII:

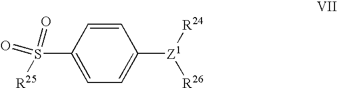

wherein:

Z¹ is selected from the group consisting of partially unsaturated or unsaturated heterocyclyl and partially unsaturated or unsaturated carbocyclic rings;

R²⁴ is selected from the group consisting of heterocyclyl, cycloalkyl, cycloalkenyl and aryl, wherein R²⁴ is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R²⁵ is selected from the group consisting of methyl or amino; and

R²⁶ is selected from the group consisting of a radical selected from H, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl, N-alkyl-N-arylaminosulfonyl;

or a prodrug thereof.

In a preferred embodiment of the invention the cyclooxygenase-2 selective inhibitor represented by the above Formula VII is selected from the group of compounds, illustrated in Table 2, which includes celecoxib (B-18), valdecoxib (B-19), deracoxib (B-20), rofecoxib (B-21), etoricoxib (MK-663; B-22), JTE-522 (B-23), or a prodrug thereof.

Additional information about selected examples of the Cox-2 selective inhibitors discussed above can be found as follows: celecoxib (CAS RN 169590-42-5, C-2779, SC-58653, and in U.S. Pat. No. 5,466,823); deracoxib (CAS RN 169590-41-4); rofecoxib (CAS RN 162011-90-7); compound B-24 (U.S. Pat. No. 5,840,924); compound B-26 (WO 00/25779); and etoricoxib (CAS RN 202409-33-4, MK-663, SC-86218, and in WO 98/03484).

TABLE 2
Examples of Tricyclic COX-2 Selective Inhibitors

| Compound | |
|---|---|
| Number | Structural Formula |
| B-18 | 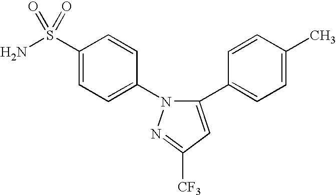 |
| B-19 | 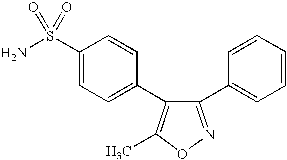 |
| B-20 | 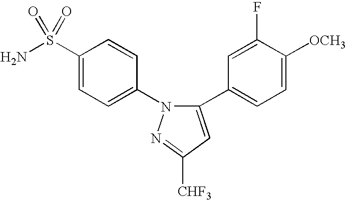 |
| B-21 | 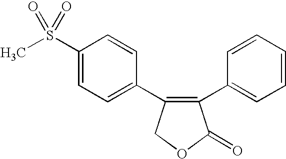 |
| B-22 | 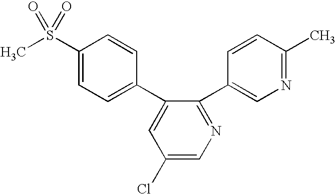 |
| B-23 | 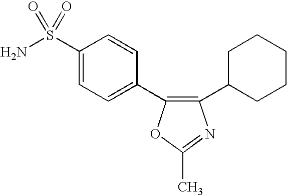 |

In a more preferred embodiment of the invention, the Cox-2 selective inhibitor is selected from the group consisting of celecoxib, rofecoxib and etoricoxib.

In a preferred embodiment of the invention, parecoxib (See, e.g. U.S. Pat. No. 5,932,598), having the structure shown in B-24, which is a therapeutically effective prodrug of the tricyclic cyclooxygenase-2 selective inhibitor valdecoxib, B-1 9, (See, e.g., U.S. Pat. No. 5,633,272), may be advantageously employed as a source of a cyclooxygenase inhibitor.

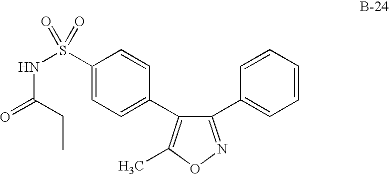

A preferred form of parecoxib is sodium parecoxib.

In another embodiment of the invention, the compound ABT-963 having the formula B-25 that has been previously described in International Publication number WO 00/24719, is another tricyclic cyclooxygenase-2 selective inhibitor which may be advantageously employed.

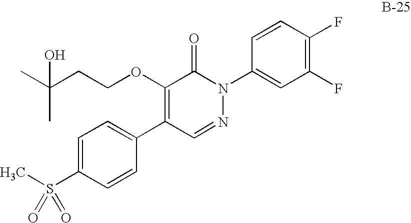

In a further embodiment of the invention, the cyclooxygenase inhibitor can be selected from the class of phenylacetic acid derivative cyclooxygenase-2 selective inhibitors represented by the general structure of Formula VIII:

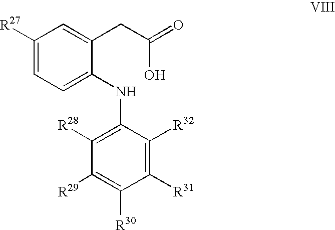

wherein:

R²⁷ is methyl, ethyl, or propyl;

R²⁸ is chloro or fluoro;

R²⁹ is hydrogen, fluoro, or methyl;

R³⁰ is hydrogen, fluoro, chloro, methyl, ethyl, methoxy, ethoxy or hydroxy;

R³¹ is hydrogen, fluoro, or methyl; and

R³² is chloro, fluoro, trifluoromethyl, methyl, or ethyl, provided that R²⁸, R²⁹, R³⁰ and R³¹ are not all fluoro when R²⁷ is ethyl and R³⁰ is H.

A phenylacetic acid derivative cyclooxygenase-2 selective inhibitor that is described in WO 99/11605 is a compound that has the structure shown in Formula VIII,

wherein:

R²⁷ is ethyl;

R²⁸ and R³⁰ are chloro;

R²⁹ and R³¹ are hydrogen; and

R³² is methyl.

Another phenylacetic acid derivative cyclooxygenase-2 selective inhibitor is a compound that has the structure shown in Formula VIII,

wherein:

R²⁷ is propyl;

R²⁸ and R³⁰ are chloro;

R²⁹ and R³¹ are methyl; and

R³² is ethyl.

Another phenylacetic acid derivative cyclooxygenase-2 selective inhibitor that is described in WO 02/20090 is a compound that is referred to as COX-189 (also termed lumiracoxib), having CAS Reg. No. 220991-20-8, and having the structure shown in Formula VIII,

wherein:

R²⁷ is methyl;

R²⁸ is fluoro;

R³² is chloro; and

R²⁹, R³⁰, and R³¹ are hydrogen.

Compounds that have a structure similar to that shown in Formula VIII, which can serve as the Cox-2 selective inhibitor of the present invention, are described in U.S. Pat. Nos. 6,310,099, 6,291,523, and 5,958,978.

Other cyclooxygenase-2 selective inhibitors that can be used in the present invention have the general structure shown in formula IX, where the J group is a carbocycle or a heterocycle. Preferred embodiments have the structure:

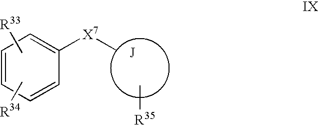

wherein:

X is O; J is 1-phenyl; R³³ is 2-NHSO₂CH₃; R³⁴ is 4-NO₂; and there is no R³⁵ group, (nimesulide), and

X is O; J is 1-oxo-inden-5-yl; R³³ is 2-F; R³⁴ is 4-F; and R³⁵ is 6-NHSO₂CH₃, (flosulide); and

X is O; J is cyclohexyl; R³³ is 2-NHSO₂CH₃; R³⁴ is 5-NO₂; and there is no R³⁵ group, (NS-398); and

X is S; J is 1-oxo-inden-5-yl; R³³ is 2-F; R³⁴ is 4-F; and R³⁵ is 6-N⁻SO₂CH₃.Na⁺, (L-745337); and

X is S; J is thiophen-2-yl; R³³ is 4-F; there is no R³⁴ group; and R³⁵ is 5-NHSO₂CH₃, (RWJ-63556); and

X is O; J is 2-oxo-5(R)-methyl-5-(2,2,2-trifluoroethyl)furan-(5H)-3-yl; R³³ is 3-F; R³⁴ is 4-F; and R³⁵ is 4-(p-SO₂CH₃)C₆H₄, (L-784512).

Further information on the applications of the Cox-2 selective inhibitor N-(2-cyclohexyloxynitrophenyl) methane sulfonamide (NS-398, CAS RN 123653-11-2), having a structure as shown in formula B-26, have been described by, for example, Yoshimi, N. et al., in Japanese J. Cancer Res., 90(4):406-412 (1999); Falgueyret, J.-P. et al., in Science Spectra, available at: http://www.gbhap.com/Science_Spectra/20-1-article.htm (Jun. 6, 2001); and Iwata, K. et al., in Jpn. J. Pharmacol., 75(2):191-194 (1997).

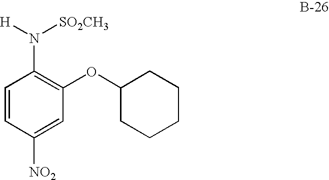

An evaluation of the anti-inflammatory activity of the cyclooxygenase-2 selective inhibitor, RWJ 63556, in a canine model of inflammation, was described by Kirchner et al., in J Pharmacol Exp Ther 282,1094-1101 (1997).

Materials that can serve as the cyclooxygenase-2 selective inhibitor of the present invention include diarylmethylidenefuran derivatives that are described in U.S. Pat. No. 6,180,651. Such diarylmethylidenefuran derivatives have the general formula shown below in formula X:

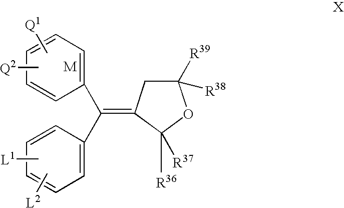

wherein:

the rings T and M independently are:

a phenyl radical,

a naphthyl radical,

a radical derived from a heterocycle comprising 5 to 6 members and possessing from 1 to 4 heteroatoms, or

a radical derived from a saturated hydrocarbon ring having from 3 to 7 carbon atoms;

at least one of the substituents Q¹, Q², L¹ or L² is:

an —S(O)ₙ—R group, in which n is an integer equal to 0, 1 or 2 and R is:

a lower alkyl radical having 1 to 6 carbon atoms or

a lower haloalkyl radical having 1 to 6 carbon atoms, or

an —SO₂NH₂ group;

and is located in the para position,

the others independently being:

a hydrogen atom,

a halogen atom,

a lower alkyl radical having 1 to 6 carbon atoms,

a trifluoromethyl radical, or

a lower O-alkyl radical having 1 to 6 carbon atoms, or

Q¹ and Q² or L¹ and L² are a methylenedioxy group; and

R^{36,} R³⁷, R³⁸ and R³⁹ independently are:

a hydrogen atom,

a halogen atom,

a lower alkyl radical having 1 to 6 carbon atoms,

a lower haloalkyl radical having 1 to 6 carbon atoms, or

an aromatic radical selected from the group consisting of phenyl, naphthyl, thienyl, furyl and pyridyl; or,

R^{36,} R³⁷ or R^{38,} R³⁹ are an oxygen atom, or

R³⁶, R³⁷ or R³⁸, R³⁹, together with the carbon atom to which they are attached, form a saturated hydrocarbon ring having from 3 to 7 carbon atoms;

or an isomer or prodrug thereof.

Particular materials that are included in this family of compounds, and which can serve as the cyclooxygenase-2 selective inhibitor in the present invention, include N-(2-cyclohexyloxynitrophenyl)methane sulfonamide, and (E)-4-[(4-methylphenyl)(tetrahydro-2-oxo-3-furanylidene) methyl]benzenesulfonamide.

Cyclooxygenase-2 selective inhibitors that are useful in the present invention include darbufelone (Pfizer), CS-502 (Sankyo), LAS 34475 (Almirall Profesfarma), LAS 34555 (Almirall Profesfarma), S-33516 (Servier), SD 8381 (Pharmacia, described in U.S. Pat. No. 6,034,256), BMS-347070 (Bristol Myers Squibb, described in U.S. Pat. No. 6,180,651), MK-966 (Merck), L-783003 (Merck), T-614 (Toyama), D-1367 (Chiroscience), L-748731 (Merck), CT3 (Atlantic Pharmaceutical), CGP-28238 (Novartis), BF-389 (Biofor/Scherer), GR-253035 (Glaxo Wellcome), 6-dioxo-9H-purin-8-yl-cinnamic acid (Glaxo Wellcome), and S-2474 (Shionogi).

Information about S-33516, mentioned above, can be found in Current Drugs Headline News, at http://www.current-drugs.com/NEWS/Inflaml.htm, Oct. 4, 2001, where it was reported that S-33516 is a tetrahydroisoinde derivative which has IC₅₀ values of 0.1 and 0.001 mM against cyclooxygenase-1 and cyclooxygenase-2, respectively. In human whole blood, S-33516 was reported to have an ED₅₀=0.39 mg/kg.

Compounds that may act as cyclooxygenase-2 selective inhibitors include multibinding compounds containing from 2 to 10 ligands covanlently attached to one or more linkers, as described in U.S. Pat. No. 6,395,724.

Compounds that may act as cyclooxygenase-2 inhibitors include conjugated linoleic acid that is described in U.S. Pat. No. 6,077,868.

Materials that can serve as a cyclooxygenase-2 selective inhibitor of the present invention include heterocyclic aromatic oxazole compounds that are described in U.S. Pat. Nos. 5,994,381 and 6,362,209. Such heterocyclic aromatic oxazole compounds have the formula shown below in formula XI:

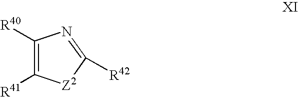

wherein:

Z² is an oxygen atom;

one of R⁴⁰ and R⁴¹ is a group of the formula

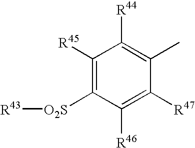

wherein:

R⁴³ is lower alkyl, amino or lower alkylamino; and

R⁴⁴, R⁴⁵, R⁴⁶ and R⁴⁷ are the same or different and each is hydrogen atom, halogen atom, lower alkyl, lower alkoxy, trifluoromethyl, hydroxy or amino, provided that at least one of R⁴⁴, R⁴⁵, R⁴⁶ and R⁴⁷ is not hydrogen atom, and the other is an optionally substituted cycloalkyl, an optionally substituted heterocyclic group or an optionally substituted aryl; and

R³⁰ is a lower alkyl or a halogenated lower alkyl, and a pharmaceutically acceptable salt thereof.

Cox-2 selective inhibitors that are useful in the subject method and compositions can include compounds that are described in U.S. Pat. Nos. 6,080,876 and 6,133,292, and described by formula XII:

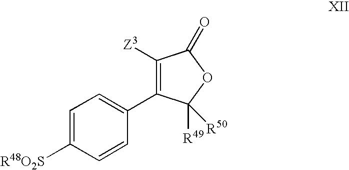

wherein:

Z³ is selected from the group consisting of:

(a) linear or branched C₁₋₆ alkyl,

(b) linear or branched C₁₋₆ alkoxy,

(c) unsubstituted, mono-, di- or tri-substituted phenyl or naphthyl wherein the substituents are selected from the group consisting of:

(1) hydrogen,

(2) halo,

(3) C₁₋₃ alkoxy,

(4) CN,

(5) C₁₋₃ fluoroalkyl

(6) C₁₋₃ alkyl,

(7) —CO₂H;

R⁴⁸ is selected from the group consisting of NH₂ and CH₃,

R⁴⁹ is selected from the group consisting of:

C₁₋₆ alkyl unsubstituted or substituted with C₃₋₆ cycloalkyl, and C₃₋₆ cycloalkyl;

R⁵⁰ is selected from the group consisting of:

C₁₋₆ alkyl unsubstituted or substituted with one, two or three fluoro atoms; and

C₃₋₆ cycloalkyl;

with the proviso that R⁴⁹ and R⁵⁰ are not the same.

Materials that can serve as cyclooxygenase-2 selective inhibitors include pyridines that are described in U.S. Pat. Nos. 6,369,275, 6,127,545, 6,130,334, 6,204,387, 6,071,936, 6,001,843 and 6,040,450, and which have the general formula described by formula XIII:

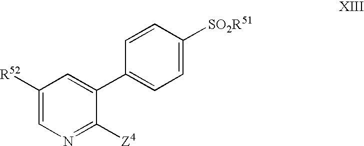

wherein:

R⁵¹ is selected from the group consisting of:

(a) CH₃,

(b) NH₂,

(c) NHC(O)CF₃,

(d) NHCH₃;

Z⁴ is a mono-, di-, or trisubstituted phenyl or pyridinyl (or the N-oxide thereof),

wherein the substituents are chosen from the group consisting of:

(a) hydrogen,

(b) halo,

(c) C₁₋₆ alkoxy,

(d) C₁₋₆ alkylthio,

(e) CN,

(f) C₁₋₆ alkyl,

(g) C₁₋₆ fluoroalkyl,

(h) N₃,

(i) —CO₂R⁵³,

(j) hydroxy,

(k) —C(R⁵⁴)(R ⁵⁵)—OH,

(l) —C₁₋₆alkyl-CO₂—R⁵⁶,

(m) C₁₋₆fluoroalkoxy;

R⁵² is chosen from the group consisting of:

(a) halo,

(b) C₁₋₆alkoxy,

(c) C₁₋₆ alkylthio,

(d) CN,

(e) C₁₋₆ alkyl,

(f) C₁₋₆ fluoroalkyl,

(g) N₃,

(h) —CO₂R⁵⁷,

(i) hydroxy,

(j) —C(R⁵⁸)(R⁵⁹)—OH,

(k) —C₁₋₆alkyl-CO₂—R⁶⁰,

(l) C₁₋₆fluoroalkoxy,

(m) NO₂,

(n) NR⁶¹R⁶², and

(o) NHCOR⁶³;

R⁵³, R⁵⁴, R⁵⁵ R⁵⁶, R⁵⁷, R⁵⁸ R⁵⁹, R⁶⁰ R⁶¹, R⁶², R⁶³, are each independently chosen from the group consisting of:

(a) hydrogen, and

(b) C₁₋₆alkyl;

or R⁵⁴ and R⁵⁵, R⁵⁸ and R⁵⁹ or R⁶¹ and R⁶² together with the atom to which they are attached form a saturated monocyclic ring of 3, 4, 5, 6, or 7 atoms.

Materials that can serve as the cyclooxygenase-2 selective inhibitor of the present invention include diarylbenzopyran derivatives that are described in U.S. Pat. No. 6,340,694. Such diarylbenzopyran derivatives have the general formula shown below in formula XIV:

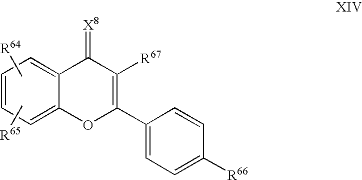

wherein:

X⁸ is an oxygen atom or a sulfur atom;

R⁶⁴ and R⁶⁵, identical to or different from each other, are independently a hydrogen atom, a halogen atom, a C₁-C₆ lower alkyl group, a trifluoromethyl group, an alkoxy group, a hydroxy group, a nitro group, a nitrile group, or a carboxyl group;

R⁶⁶ is a group of a formula: S(O)ₙR⁶⁸ wherein n is an integer of 0˜2, R⁶⁸ is a hydrogen atom, a C₁-C₆ lower alkyl group, or a group of a formula: NR⁶⁹ R⁷⁰ wherein R⁶⁹ and R⁷⁰, identical to or different from each other, are independently a hydrogen atom, or a C₁-C₆ lower alkyl group; and

R⁶⁷ is oxazolyl, benzo[b]thienyl, furanyl, thienyl, naphthyl, thiazolyl, indolyl, pyrolyl, benzofuranyl, pyrazolyl, pyrazolyl substituted with a C₁-C₆ lower alkyl group, indanyl, pyrazinyl, or a substituted group represented by the following structures:

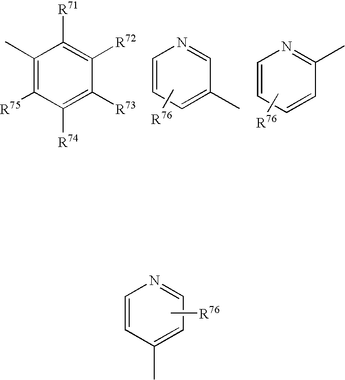

wherein:

R⁷¹ through R⁷⁵, identical to or different from one another, are independently a hydrogen atom, a halogen atom, a C₁-C₆ lower alkyl group, a trifluoromethyl group, an alkoxy group, a hydroxy group, a hydroxyalkyl group, a nitro group, a group of a formula: S(O)ₙR⁶⁸, a group of a formula: NR⁶⁹R⁷⁰, a trifluoromethoxy group, a nitrile group a carboxyl group, an acetyl group, or a formyl group,

wherein n, R⁶⁸, R⁶⁹ and R⁷⁰ have the same meaning as defined by R⁶⁶ above; and

R⁷⁶ is a hydrogen atom, a halogen atom, a C₁-C₆ lower alkyl group, a trifluoromethyl group, an alkoxy group, a hydroxy group, a trifluoromethoxy group, a carboxyl group, or an acetyl group.

Materials that can serve as the cyclooxygenase-2 selective inhibitor of the present invention include 1-(4-sulfamylaryl)-3-substituted-5-aryl-2-pyrazolines that are described in U.S. Pat. No. 6,376,519. Such 1-(4-sulfamylaryl)-3-substituted-5-aryl-2-pyrazolines have the formula shown below in formula XV:

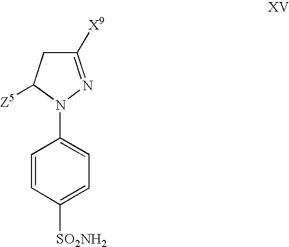

wherein:

X⁹ is selected from the group consisting of C₁-C₆ trihalomethyl, preferably trifluoromethyl; C₁-C₆ alkyl; and an optionally substituted or di-substituted phenyl group of formula XVI:

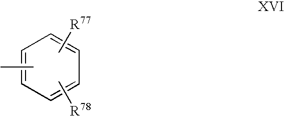

wherein:

R⁷⁷ and R⁷⁸ are independently selected from the group consisting of hydrogen, halogen, preferably chlorine, fluorine and bromine; hydroxyl; nitro; C₁-C₆ alkyl, preferably C₁-C₃ alkyl; C₁-C₆ alkoxy, preferably C₁-C₃ alkoxy; carboxy; C₁-C₆ trihaloalkyl, preferably trihalomethyl, most preferably trifluoromethyl; and cyano;

Z⁵ is selected from the group consisting of substituted and unsubstituted aryl.

Materials that can serve as the cyclooxygenase-2 selective inhibitor of the present invention include heterocycles that are described in U.S. Pat. No. 6,153,787. Such heterocycles have the general formulas shown below in formulas XVII and XVIII:

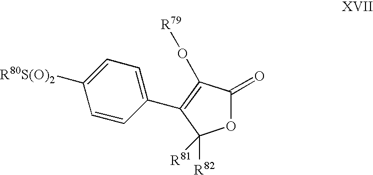

wherein:

R⁷⁹ is a mono-, di-, or tri-substituted C₁₋₁₂ alkyl, or a mono-, or an unsubstituted or mono-, di- or tri-substituted linear or branched C₂₋₁₀ alkenyl, or an unsubstituted or mono-, di- or tri-substituted linear or branched C₂₋₁₀ alkynyl, or an unsubstituted or mono-, di- or tri-substituted C₃₋₁₂ cycloalkenyl, or an unsubstituted or mono-, di- or tri-substituted C₅₋₁₂ cycloalkynyl, wherein the substituents are chosen from the group consisting of:

(a) halo, selected from F, Cl, Br, and I,

(b) OH,

(c) CF₃,

(d) C₃₋₆ cycloalkyl,

(e) ═O,

(f) dioxolane,

(g) CN; and

R⁸⁰ is selected from the group consisting of:

(a) CH₃,

(b) NH₂,

(c) NHC(O) CF₃,

(d) NHCH₃;

R⁸¹ and R⁸² are independently chosen from the group consisting of:

(a) hydrogen,

(b) C₁₋₁₀ alkyl;

or R⁸¹ and R⁸² together with the carbon to which they are attached form a saturated monocyclic carbon ring of 3, 4, 5, 6 or 7 atoms.

Formula XVIII is:

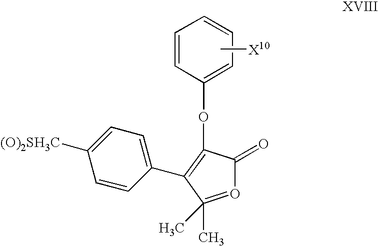

X¹⁰ is fluoro or chloro.

Materials that can serve as the cyclooxygenase-2 selective inhibitor of the present invention include 2,3,5-trisubstituted pyridines that are described in U.S. Pat. No. 6,046,217. Such pyridines have the general formula shown below in formula XIX:

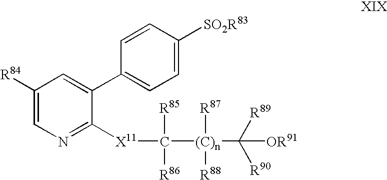

or a pharmaceutically acceptable salt thereof,

wherein:

X¹¹ is selected from the group consisting of:

(a) O,

(b) S,

(c) bond;

n is 0 or 1;

R⁸³ is selected from the group consisting of:

(a) CH₃,

(b) NH₂,

(c) NHC(O)CF₃;

R⁸⁴ is chosen from the group consisting of:

(a) halo,

(b) C₁₋₆ alkoxy,

(c) C₁₋₆ alkylthio,

(d) CN,

(e) C₁₋₆ alkyl,

(f) C₁₋₆ fluoroalkyl,

(g) N₃,

(h) —CO₂R⁹²,

(i) hydroxy,

(j) —C(R⁹³)(R⁹⁴)—OH,

(k) —C₁₋₆ alkyl-CO₂—R⁹⁵,

(l) C₁-6 fluoroalkoxy,

(m) NO₂,

(n) NR⁹⁶R⁹⁷,

(o) NHCOR⁹⁸;

R⁸⁵ to R⁹⁸ are independantly chosen from the group consisting of

(a) hydrogen,

(b) C₁₋₆ alkyl;

or R⁸⁵ and R⁸⁹, or R⁸⁹ and R⁹⁰ together with the atoms to which they are attached form a carbocyclic ring of 3, 4, 5, 6 or 7 atoms, or R⁸⁵ and R⁸⁷ are joined to form a bond.

One preferred embodiment of the Cox-2 selective inhibitor of formula XIX is that wherein X is a bond.

Another preferred embodiment of the Cox-2 selective inhibitor of formula XIX is that wherein X is O.

Another preferred embodiment of the Cox-2 selective inhibitor of formula XIX is that wherein X is S.

Another preferred embodiment of the Cox-2 selective inhibitor of formula XIX is that wherein R⁸³ is CH₃.

Another preferred embodiment of the Cox-2 selective inhibitor of formula XIX is that wherein R⁸⁴ is halo or C₁₋₆ fluoroalkyl.

Materials that can serve as the cyclooxygenase-2 selective inhibitor of the present invention include diaryl bicyclic heterocycles that are described in U.S. Pat. No. 6,329,421. Such diaryl bicyclic heterocycles have the general formula shown below in formula XX:

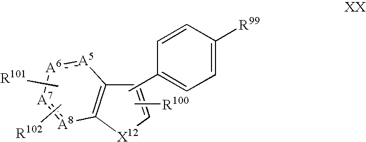

and pharmaceutically acceptable salts thereof wherein:

-A⁵=A⁶-A⁷=A⁸- is selected from the group consisting of:

(a) —CH═CH—CH═CH—,

(b) —CH₂—CH₂—CH₂—C(O)—, —CH₂—CH₂—C(O)—CH₂—, —CH₂—C(O)—CH₂—CH₂, —C(O)—CH₂—CH₂—CH₂,

(c) —CH₂—CH₂—C(O)—, —CH₂—C(O)—CH₂—, —C(O)—CH₂—CH₂—

(d) —CH₂—CH₂—O—C(O)—, CH₂—O—C(O)—CH₂—, —O—C(O)—CH₂—CH₂—,

(e) —CH₂—CH₂—C(O)—O—, —CH₂—C(O)—OCH₂—C(O)—O—CH₂—CH₂—,

(f) —C(R¹⁰⁵)₂—O—C(O)—, —C(O)—O—C(R¹⁰⁵)₂—O—C(O)—C(R¹⁰⁵)₂—, —C(R¹⁰⁵)₂—C(O)—O—,

(g) —N═CH—CH═CH—,

(h) —CH═N—CH═CH—,

(i) —CH═CH—N═CH—,

(j) —CH═CH—CH═N—,

(k) —N═CH—CH═N—,

(l) —N═CH—N═CH—,

(m) —CH═N—CH═N—,

(n) —S—CH═N—,

(o) —S—N═CH—,

(p) —N═N—NH—,

(q) —CH═N—S—, and

(r) —N═CH—S—;

R⁹⁹ is selected from the group consisting of:

(a) S(O)₂CH₃,

(b) S(O)₂NH₂,

(c) S(O)₂NHCOCF₃,

(d) S(O)(NH)CH₃,

(e) S(O)(NH)NH₂,

(f) S(O)(NH)NHCOCF₃,

(g) P(O)(CH₃)OH, and

(h) P()(CH₃)NH₂;

R¹⁰⁰ is selected from the group consisting of:

(a) C₁₋₆ alkyl,

(b) C_{3-7,} cycloalkyl,

(c) mono- or di-substituted phenyl or naphthyl wherein the substituent is selected from the group consisting of:

(1) hydrogen,

(2) halo, including F, Cl, Br, I,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹⁰³)(R¹⁰⁴)—OH,

(12) —C(R¹⁰³)(R¹⁰⁴)—O—C₁₋₄ alkyl, and

(13) —C₁₋₆ alkyl-CO₂—R¹⁰⁶

(d) mono- or di-substituted heteroaryl wherein the heteroaryl is a monocyclic aromatic ring of 5 atoms, said ring having one hetero atom which is S, O, or N, and optionally 1, 2, or 3 additional N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹⁰³)(R¹⁰⁴)—OH, and

(10) —C(R¹⁰³)(R¹⁰⁴)—OC₁₋₄ alkyl;

(e) benzoheteroaryl which includes the benzo fused analogs of (d);

R¹⁰¹ and R¹⁰² are the substituents residing on any position of -A⁵=A⁶-A⁷=A⁸- and are selected independently from the group consisting of:

(a) hydrogen,

(b) CF₃,

(c) CN,

(d) C₁₋₆ alkyl,

(e) -Q³ wherein Q³ is Q⁴, CO₂H, C(R¹⁰³)(R¹⁰⁴)OH,

(f) —O-Q⁴,

(g) —S-Q⁴, and

(h) optionally substituted:

(1) —C₁₋₅ alkyl-Q³,

(2) —O—C₁₋₅ alkyl-Q³,

(3) —S—C₁₋₅ alkyl-Q³,

(4) —C₁₋₃ alkyl-O—C_{1 3} alkyl-Q³,

(5) —C₁₋₃ alkyl-S—C₁₋₃ alkyl-Q³,

(6) —C₁₋₅ alkyl-O-Q⁴,

(7) —C₁₋₅ alkyl-S-Q⁴,

wherein the substituent resides on the alkyl chain and the substituent is C₁₋₃ alkyl, and Q³ is Q⁴, CO₂H, C(R¹⁰³)(R¹⁰⁴)OH Q⁴ is CO₂—C₁₋₄ alkyl, tetrazolyl-5-yl, or C(R¹⁰³)(R¹⁰⁴)O—C₁₋₄ alkyl;

R¹⁰³, R¹⁰⁴ and R¹⁰⁵ are each independently selected from the group consisting of

(a) hydrogen,

(b) C₁₋₆ alkyl; or

R¹⁰³ and R¹⁰⁴ together with the carbon to which they are attached form a saturated monocyclic carbon ring of 3, 4, 5, 6 or 7 atoms, or two R¹⁰⁵ groups on the same carbon form a saturated monocyclic carbon ring of 3, 4, 5, 6 or 7 atoms;

R¹⁰⁶ is hydrogen or C₁₋₆ alkyl;

R¹⁰⁷ is hydrogen, C₁₋₆ alkyl or aryl;

X⁷ is O, S, NR¹⁰⁷, CO, C(R¹⁰⁷)₂, C(R¹⁰⁷)(OH), —C(R¹⁰⁷)═C(R¹⁰⁷)—; —C(R¹⁰⁷)═N—; —N═C(R¹⁰⁷)—.

Compounds that may act as cyclooxygenase-2 inhibitors include salts of 5-amino or a substituted amino 1,2,3-triazole compound that are described in U.S. Pat. No. 6,239,137. The salts are of a class of compounds of formula XXI:

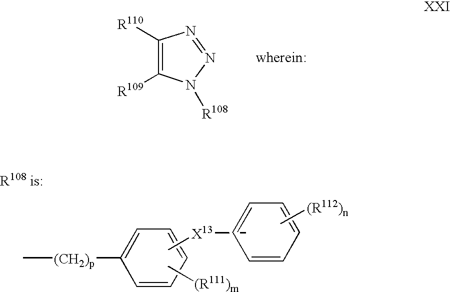

wherein:

p is 0 to 2; m is 0 to 4; and n is 0 to 5; X¹³ is O, S, SO, SO₂, CO, CHCN, CH₂ or C═NR¹¹³ where R¹¹³ is hydrogen, loweralkyl, hydroxy, loweralkoxy, amino, loweralkylamino, diloweralkylamino or cyano; and, R¹¹¹ and R¹¹² are independently halogen, cyano, trifluoromethyl, loweralkanoyl, nitro, loweralkyl, loweralkoxy, carboxy, lowercarbalkoxy, trifuloromethoxy, acetamido, loweralkylthio, loweralkylsulfinyl, loweralkylsulfonyl, trichlorovinyl, trifluoromethylthio, trifluoromethylsulfinyl, or trifluoromethylsulfonyl; R¹⁰⁹ is amino, mono or diloweralkyl amino, acetamido, acetimido, ureido, formamido, formamido or guanidino; and R¹¹⁰ is carbamoyl, cyano, carbazoyl, amidino or N-hydroxycarbamoyl; wherein the loweralkyl, loweralkyl containing, loweralkoxy and loweralkanoyl groups contain from 1 to 3 carbon atoms.

Materials that can serve as a cyclooxygenase-2 selective inhibitor of the present invention include pyrazole derivatives that are described in U.S. Pat. No. 6,136,831. Such pyrazole derivatives have the formula shown below in formula XXII:

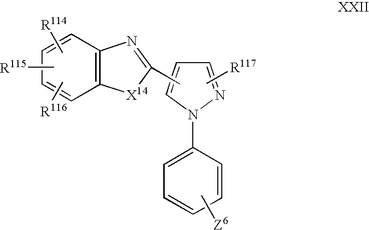

wherein:

R¹¹⁴ is hydrogen or halogen, R¹¹⁵ and R¹¹⁶ are each independently hydrogen, halogen, lower alkyl, lower alkoxy, hydroxy or lower alkanoyloxy;

R¹¹⁷ is lower haloalkyl or lower alkyl;

X¹⁴ is sulfur, oxygen or NH; and

Z⁶ is lower alkylthio, lower alkylsulfonyl or sulfamoyl; or a pharmaceutically acceptable salt thereof.

Materials that can serve as a cyclooxygenase-2 selective inhibitor of the present invention include substituted derivatives of benzosulphonamides that are described in U.S. Pat. No. 6,297,282. Such benzosulphonamide derivatives have the formula shown below in formula XXIII:

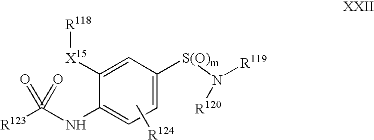

wherein:

X¹⁵ denotes oxygen, sulphur or NH;

R¹¹⁸ is an optionally unsaturated alkyl or alkyloxyalkyl group, optionally mono- or polysubstituted or mixed substituted by halogen, alkoxy, oxo or cyano, a cycloalkyl, aryl or heteroaryl group optionally mono- or polysubstituted or mixed substituted by halogen, alkyl, CF₃, cyano or alkoxy;

R¹¹⁹ and R¹²⁰, independently from one another, denote hydrogen, an optionally polyfluorised alkyl group, an aralkyl, aryl or heteroaryl group or a group (CH₂)ₙ—X¹⁶; or

R¹¹⁹ and R¹²⁰, together with the N-atom, denote a 3 to 7-membered, saturated, partially or completely unsaturated heterocycle with one or more heteroatoms N, O or S, which can optionally be substituted by oxo, an alkyl, alkylaryl or aryl group, or a group (CH₂)ₙ—X¹⁶;

X¹⁶ denotes halogen, NO₂, —OR¹²¹, —COR¹²¹, —CO₂ R¹²¹, —OCO₂ R¹²¹, —CN, —CONR¹²¹OR¹²², —CONR¹²¹R¹²², —SR¹²¹, —S(O)R¹²¹, —S(O)₂ R¹²¹, —NR¹²¹R¹²², —NHC(O)R¹²¹, —NHS(O)₂R¹²¹;

n denotes a whole number from 0 to 6;

R¹²³ denotes a straight-chained or branched alkyl group with 1-10 C-atoms, a cycloalkyl group, an alkylcarboxyl group, an aryl group, aralkyl group, a heteroaryl or heteroaralkyl group which can optionally be mono- or polysubstituted or mixed substituted by halogen or alkoxy;

R¹²⁴ denotes halogen, hydroxy, a straight-chained or branched alkyl, alkoxy, acyloxy or alkyloxycarbonyl group with 1-6 C-atoms, which can optionally be mono- or polysubstituted by halogen, NO₂, —OR¹²¹, —COR¹²¹, —CO₂R¹²¹, —OCO₂R¹²¹, —CN, —CONR¹²¹OR¹²², —CONR¹²¹R¹²², —SR¹²¹, S(O)R¹²¹, —S(O)₂R¹²¹, —NR¹²¹R¹²², —NHC(O)R¹²¹, —NHS(O)₂R¹²¹, or a polyfluoroalkyl group;

R¹²¹ and R¹²², independently from one another, denote hydrogen, alkyl, aralkyl or aryl; and

m denotes a whole number from 0 to 2;

and the pharmaceutically-acceptable salts thereof.

Materials that can serve as a cyclooxygenase-2 selective inhibitor of the present invention include 3-phenyl-4-(4(methylsulfonyl)phenyl)-2-(5H)-furanones that are described in U.S. Pat. No. 6,239,173. Such 3-phenyl-4-(4(methylsulfonyl)phenyl)-2-(5H)-furanones have the formula shown below in formula XXIV:

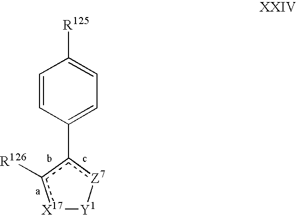

or pharmaceutically acceptable salts thereof wherein:

X¹⁷—Y¹-Z⁷—is selected from the group consisting of:

(a) —CH₂CH₂CH₂—,

(b) —C(O)CH₂CH₂—,

(c) —CH₂CH₂C(O)—,

(d) —CR¹²⁹(R^{129′})—O—C(O)—,

(e) —C(O)—O—CR¹²⁹(R^{129′})—,

(f) —CH₂—NR¹²⁷—CH₂—,

(g) —CR¹²⁹(R^{129′})—NR¹²⁷—C(O)—,

(h) —CR¹²⁸═CR^{128′}—S—,

(i) —S—CR¹²⁸═CR^{128′}—,

(j) —S—N═CH—,

(k) —CH═N—S—,

(l) —N═CR¹²⁸—O—,

(m) —O—CR4=N—,

(n) —N═CR¹²⁸—NH—,

(o) —N═CR¹²⁸—S—, and

(p) —S—CR¹²⁸═N—,

(q) —C(O)—NR¹²⁷—CR¹²⁹(R^{129′})—,

(r) —R¹²⁷N—CH═CH— provided R₁₂₂ is not —S(O)₂CH₃,

(s) —CH═CH—NR¹²⁷— provided R¹²⁵ is not —S(O)₂CH₃,

when side b is a double bond, and sides a and c are single bonds; and

X¹⁷—Y¹-Z⁷— is selected from the group consisting of:

(a) ═CH—O—CH═, and

(b) ═CH—NR¹²⁷—CH═,

(c) ═N—S—CH═,

(d) ═CH—S—N═,

(e) ═N—O—CH═,

(f) ═CH—O—N═,

(g) ═N—S—N═,

(h) ═N—O—N═,

when sides a and c are double bonds and side b is a single bond;

R¹²⁵ is selected from the group consisting of:

(a) S(O)₂CH₃,

(b) S(O)₂NH₂,

(c) S(O)₂NHC(O)CF₃,

(d) S(O)(NH)CH₃,

(e) S(O)(NH)NH₂,

(f) S(O)(NH)NHC(O)CF₃,

(g) P(O)(CH₃)OH, and

(h) P(O)(CH₃)NH₂;

R¹²⁶ is selected from the group consisting of

(a) C₁₋₆ alkyl,

(b) C₃, C₄, C₅, C₆, and C₇, cycloalkyl,

(c) mono-, di- or tri-substituted phenyl or naphthyl,

wherein the substituent is selected from the group consisting of:

(1) hydrogen,

(2) halo,

(3) C₁₋₆ alkoxy,

(4) C₁₋₆ alkylthio,

(5) CN,

(6) CF₃,

(7) C₁₋₆ alkyl,

(8) N₃,

(9) —CO₂H,

(10) —CO₂—C₁₋₄ alkyl,

(11) —C(R¹²⁹)(R¹³⁰)—OH,

(12) —C(R¹²⁹)(R¹³⁰)—O—C₁₋₄ alkyl, and

(13) —C₁₋₆ alkyl-CO₂—R¹²⁹;

(d) mono-, di- or tri-substituted heteroaryl wherein the heteroaryl is a monocyclic aromatic ring of 5 atoms, said ring having one hetero atom which is S, O, or N, and optionally 1, 2, or 3 additionally N atoms; or the heteroaryl is a monocyclic ring of 6 atoms, said ring having one hetero atom which is N, and optionally 1, 2, 3, or 4 additional N atoms; said substituents are selected from the group consisting of:

(1) hydrogen,

(2) halo, including fluoro, chloro, bromo and iodo,

(3) C₁₋₆ alkyl,

(4) C₁₋₆ alkoxy,

(5) C₁₋₆ alkylthio,

(6) CN,

(7) CF₃,

(8) N₃,

(9) —C(R¹²⁹)(R¹³⁰)—OH, and

(10) —C(R¹²⁹)(R¹³⁰)—O—C₁₋₄ alkyl;

(e) benzoheteroaryl which includes the benzo fused analogs of (d);

R¹²⁷ is selected from the group consisting of:

(a) hydrogen,

(b) CF₃,

(c) CN,

(d) C₁₋₆ alkyl,

(e) hydroxyC₁₋₆ alkyl,

(f) —C(O)—C₁₋₆ alkyl,

(g) optionally substituted:

(1) —C₁₋₅ alkyl-Q⁵,

(2) —C₁₋₃ alkyl-O—C₁₋₃ alkyl-Q⁵,

(3) —C₁₋₃ alkyl-S—C₁₋₃ alkyl-Q⁵,

(4) —C₁₋₅ alkyl-O-Q⁵, or

(5) —C₁₋₅ alkyl-S-Q⁵,

wherein the substituent resides on the alkyl and the substituent is

C₁₋₃ alkyl;

(h) -Q⁵;

R¹²⁸ and R^{128′} are each independently selected from the group consisting of:

(a) hydrogen,

(b) CF₃,

(c) CN,

(d) C₁₋₆ alkyl,

(e) -Q⁵,

(f) —O-Q⁵;

(g) —S-Q⁵, and

(h) optionally substituted:

(1) —C₁₋₅ alkyl-Q⁵,

(2) —O—C₁₋₅ alkyl-Q⁵,

(3) —S—C₁₋₅ alkyl-Q⁵,

(4) —C₁₋₃ alkyl-O—C₁₋₃ alkyl-Q⁵,

(5) —C₁₋₃ alkyl-S—C₁₋₃ alkyl-Q⁵,

(6) —C₁₋₅ alkyl-O-Q⁵,

(7) —C₁₋₅ alkyl-S-Q⁵,

wherein the substituent resides on the alkyl and the substituent is

C₁₋₃ alkyl, and

R¹²⁹, R^{129′}, R¹³⁰, R¹³¹ and R¹³² are each independently selected from the group consisting of:

(a) hydrogen,

(b) C₁₋₆ alkyl;

or R¹²⁹ and R¹³⁰ or R¹³¹ and R¹³² together with the carbon to which they are attached form a saturated monocyclic carbon ring of 3, 4, 5, 6 or 7 atoms;

Q⁵ is CO₂H, CO₂—C₁₋₄ alkyl, tetrazolyl-5-yl, C(R¹³¹)(R¹³²)(OH), or C(R¹³¹)(R¹³²)(O—C₁₋₄ alkyl);

provided that when X—Y-Z is —S—CR¹²⁸═CR^{128′}, then R¹²⁸ and R^{128′} are other than CF₃.

Materials that can serve as a cyclooxygenase-2 selective inhibitor of the present invention include bicycliccarbonyl indole compounds that are described in U.S. Pat. No. 6,303,628. Such bicycliccarbonyl indole compounds have the formula shown below in formula XXV:

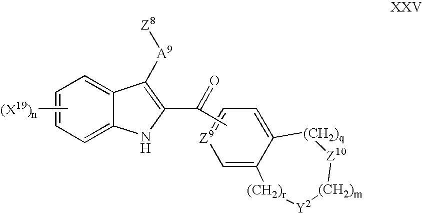

or the pharmaceutically acceptable salts thereof wherein

A⁹ is C₁₋₆ alkylene or —NR¹³³—;

Z⁸ is C(=L³)R¹³⁴, or SO₂R¹³⁵;

Z⁹ is CH or N;

Z¹⁰ and Y² are independently selected from —CH₂—, O, S and —N—R¹³³;

m is 1, 2 or 3;

q and r are independently 0, 1 or 2;

X¹⁸ is independently selected from halogen, C₁₋₄ alkyl, halo-substituted C₁₋₄ alkyl, hydroxy, C₁₋₄ alkoxy, halo-substituted C₁₋₄ alkoxy, C₁₋₄ alkylthio, nitro, amino, mono- or di-(C₁₋₄ alkyl)amino and cyano;

n is 0, 1, 2, 3 or 4;

L³ is oxygen or sulfur;

R¹³³ is hydrogen or C₁₋₄ alkyl;

R¹³⁴ is hydroxy, C₁₋₆ alkyl, halo-substituted C₁₋₆ alkyl, C₁₋₆ alkoxy, halo-substituted C₁₋₆ alkoxy, C₃₋₇ cycloalkoxy, C₁₋₄ alkyl(C₃₋₇ cycloalkoxy), —NR¹³⁶R¹³⁷, C₁₋₄ alkylphenyl-O— or phenyl-O—, said phenyl being optionally substituted with one to five substituents independently selected from halogen, C₁₋₄ alkyl, hydroxy, C₁₋₄ alkoxy and nitro;

R¹³⁵ is C₁₋₆ alkyl or halo-substituted C₁₋₆ alkyl; and

R¹³⁶ and R¹³⁷ are independently selected from hydrogen, C₁₋₆ alkyl and halo-substituted C₁₋₆ alkyl.

Materials that can serve as a cyclooxygenase-2 selective inhibitor of the present invention include benzimidazole compounds that are described in U.S. Pat. No. 6,310,079. Such benzimidazole compounds have the formula shown below in formula XXVI:

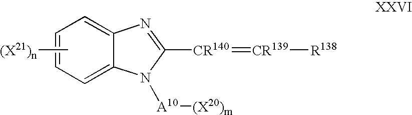

or a pharmaceutically acceptable salt thereof, wherein:

A¹⁰ is heteroaryl selected from a 5-membered monocyclic aromatic ring having one hetero atom selected from O, S and N and optionally containing one to three N atom(s) in addition to said hetero atom, or a 6-membered monocyclic aromatic ring having one N atom and optionally containing one to four N atom(s) in addition to said N atom; and said heteroaryl being connected to the nitrogen atom on the benzimidazole through a carbon atom on the heteroaryl ring;

X²⁰ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N-(C₁-C₄ alkyl)amino, N, N-di(C₁-C₄ alkyl)amino, [N—(C₁-C₄ alkyl)amino]C₁-C₄ alkyl, [N, N-di(C₁-C₄ alkyl)amino]C₁-C₄ alkyl, N—(C₁-C₄ alkanoyl)amonio, N—(C₁-C₄ alkyl)(C₁-C₄ alkanoyl)amino, N—[(C₁-C₄ alkyl)sulfonyl]amino, N—[(halo-substituted C₁-C₄ alkyl)sulfonyl]amino, C₁-C₄ alkanoyl, carboxy, (C₁-C₄ alkoxy)carbonyl, carbamoyl, [N—(C₁-C₄ alkyl)amino]carbonyl, [N, N-di(C₁-C₄ alkyl)amino]carbonyl, cyano, nitro, mercapto, (C₁-C₄ alkyl)thio, (C₁-C₄ alkyl)sulfinyl, (C₁ -C₄ alkyl)sulfonyl, aminosulfonyl, [N—(C₁-C₄ alkyl)amino]sulfonyl and [N, N-di(C₁-C₄ alkyl)amino]sulfonyl;

X²¹ is independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N, N-di(C₁-C₄ alkyl)amino, [N-(C₁-C₄ alkyl)amino]C₁-C₄ alkyl, [N, N-di(C₁-C₄ alkyl)amino]C₁-C₄ alkyl, N—(C₁-C₄ alkanoyl)amino, N—(C₁-C₄ alkyl)-N—(C₁-C₄ alkanoyl) amino, N—[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)cabonyl, cabamoyl, [N—(C₁-C₄ alkyl) amino]carbonyl, [N, N-di(C₁-C₄ alkyl)amino]carbonyl, N-carbomoylamino, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N, N-di(C₁-C₄ alkyl)amino]sulfonyl;

R¹³⁸ is selected from hydrogen,

straight or branched C₁-C₄ alkyl optionally substituted with one to three substituent(s) wherein said substituents are independently selected from halo hydroxy, C₁-C₄ alkoxy, amino, N—(C₁-C₄ alkyl)amino and N, N-di(C₁-C₄ alkyl)amino,

C₃-C₈ cycloalkyl optionally substituted with one to three substituent(s) wherein said substituents are indepently selected from halo, C₁-C₄ alkyl, hydroxy, C₁-C₄ alkoxy, amino, N—(C₁-C₄ alkyl)amino and N, N-di(C₁-C₄ alkyl)amino,

C₄-C₈ cycloalkenyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N, N-di(C₁-C₄ alkyl)amino,

phenyl optionally substituted with one to three substituent(s) wherein said substituents are independently selected from halo, C₁-C₄ alkyl, hydroxy, C₁-C₄ alkoxy, halo-substituted C₁-C₄ alkyl, hydroxy-substituted C₁-C₄ alkyl, (C₁-C₄ alkoxy)C₁-C₄ alkyl, halo-substituted C₁-C₄ alkoxy, amino, N—(C₁-C₄ alkyl)amino, N, N-di(C₁-C₄ alkyl)amino, [N—(C₁-C₄ alkyl)amino]C₁-C₄ alkyl, [N, N-di(C₁-C₄ alkyl)amino]C₁-C₄ alkyl, N—(C₁-C₄ alkanoyl)amino, N—[C₁-C₄ alkyl)(C₁-4 alkanoyl)]amino, N—[(C₁-C₄ alkyl)sulfonyl]amino, N-[(halo-substituted C₁-C₄ alkyl)sulfonyl]amino, C₁-C₄ alkanoyl, carboxy, (C₁-C₄ alkoxy)carbonyl, carbomoyl, [N—(C₁-C₄ alky)amino]carbonyl, [N, N-di(C₁-C₄ alkyl)amino]carbonyl, cyano, nitro, mercapto, (C₁-C₄ alkyl)thio, (C₁-C₄ alkyl)sulfinyl, (C₁-C₄ alkyl)sulfonyl, aminosulfonyl, [N—(C₁-C₄ alkyl)amino]sulfonyl and [N, N-di(C₁-C₄ alkyl)amino]sulfonyl; and

heteroaryl selected from:

a 5-membered monocyclic aromatic ring having one hetero atom selected from O, S and N and optionally containing one to three N atom(s) in addition to said hetero atom; or a 6-membered monocyclic aromatic ring having one N atom and optionally containing one to four N atom(s) in addition to said N atom; and

said heteroaryl being optionally substituted with one to three substituent(s) selected from X²⁰;

R¹³⁹ and R¹⁴⁰ are independently selected from:

hydrogen,

halo,

C₁-C₄ alkyl,

phenyl optionally substituted with one to three substituent(s) wherein said substituents are independently selected from halo, C₁-C₄ alkyl, hydroxy, C₁-C₄ alkoxy, amino, N—(C₁-C₄ alkyl)amino and N, N-di(C₁-C₄ alkyl)amino,

or R¹³⁸ and R¹³⁹ can form, together with the carbon atom to which they are attached, a C₃ -C₇ cycloalkyl ring;

m is 0, 1, 2, 3, 4 or 5; and

n is 0, 1, 2, 3 or 4.

Materials that can serve as a cyclooxygenase-2 selective inhibitor of the present invention include indole compounds that are described in U.S. Pat. No. 6,300,363. Such indole compounds have the formula shown below in formula XXVII:

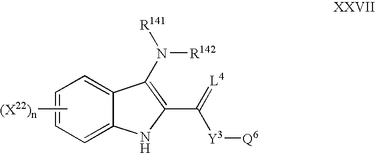

and the pharmaceutically acceptable salts thereof,

wherein:

L⁴ is oxygen or sulfur;

Y³ is a direct bond or C₁₋₄ alkylidene;

Q⁶ is:

(a) C₁₋₆ alkyl or halosubstituted C₁₋₆ alkyl, said alkyl being optionally substituted with up to three substituents independently selected from hydroxy, C₁₋₄ alkoxy, amino and mono- or di-(C₁₋₄ alkyl)amino,

(b) C₃₋₇ cycloalkyl optionally substituted with up to three substituents independently selected from hydroxy, C₁₋₄ alkyl and C₁₋₄ alkoxy,

(c) phenyl or naphthyl, said phenyl or naphthyl being optionally substituted with up to four substituents independently selected from:

(c-1) halo, C₁₋₄ alkyl, halosubstituted C₁₋₄ alkyl, hydroxy, C₁₋₄ alkoxy, halosubstituted C₁₋₄ alkoxy, S(O)ₘ R¹⁴³, SO₂NH₂, SO₂ N(C₁₋₄ alkyl)₂, amino, mono- or di-(C₁₋₄ alkyl)amino, NHSO₂R¹⁴³, NHC(O)R¹⁴³, CN, CO₂ H, CO₂ (C₁₋₄ alkyl), C₁₋₄ alkyl-OH, C₁₋₄ alkyl-OR¹⁴³, CONH₂, CONH(C₁₋₄ alkyl), CON(C₁₋₄ alkyl)₂ and —O—Y-phenyl, said phenyl being optionally substituted with one or two substituents independently selected from halo, C₁₋₄ alkyl, CF₃, hydroxy, OR¹⁴³, S(O)ₘR¹⁴³, amino, mono- or di-(C₁₋₄ alkyl)amino and CN;

(d) a monocyclic aromatic group of 5 atoms, said aromatic group having one heteroatom selected from O, S and N and optionally containing up to three N atoms in addition to said heteroatom, and said aromatic group being substituted with up to three substituents independently selected from:

(d-1) halo, C₁₋₄ alkyl, halosubstituted C₁₋₄ alkyl, hydroxy, C₁₋₄ alkoxy, halosubstituted C₁₋₄ alkoxy, C₁₋₄ alkyl-OH, S(O)ₘR¹⁴³, SO₂NH₂, SO₂ N(C₁₋₄ alkyl)₂, amino, mono- or di-(C₁₋₄ alkyl)amino, NHSO₂R¹⁴³, NHC(O)R¹⁴³, CN, CO₂H, CO₂ (C₁₋₄ alkyl), C₁₋₄ alkyl-OR¹⁴³, CONH₂, CONH(C₁₋₄ alkyl), CON(C₁₋₄ alkyl)₂, phenyl, and mono-, di- or tri-substituted phenyl wherein the substituent is independently selected from halo, CF₃, C₁₋₄ alkyl, hydroxy, C₁₋₄ alkoxy, OCF₃, SR¹⁴³, SO₂CH₃, SO₂NH₂, amino, C₁₋₄ alkylamino and NHSO₂R¹⁴³;

(e) a monocyclic aromatic group of 6 atoms, said aromatic group having one heteroatom which is N and optionally containing up to three atoms in addition to said heteroatom, and said aromatic group being substituted with up to three substituents independently selected from the above group (d-1);

R¹⁴¹ is hydrogen or C₁₋₆ alkyl optionally substituted with a substituent selected independently from hydroxy, OR¹⁴³, nitro, amino, mono- or di-(C₁₋₄ alkyl)amino, CO₂H, CO₂ (C₁₋₄ alkyl), CONH₂, CONH(C₁₋₄ alkyl) and CON(C₁₋₄ alkyl)₂;

R¹⁴² is:

(a) hydrogen,

(b) C₁₋₄ alkyl,

(c) C(O)R¹⁴⁵,

wherein R¹⁴⁵ is selected from:

(c-1) C₁₋₂₂ alkyl or C₂₋₂₂ alkenyl, said alkyl or alkenyl being optionally substituted with up to four substituents independently selected from:

(c-1-1) halo, hydroxy, OR¹⁴³, S(O)ₘR¹⁴³, nitro, amino, mono- or di-(C₁₋₄ alkyl)amino, NHSO₂R¹⁴³, CO₂H, CO₂ (C₁₋₄ alkyl), CONH₂, CONH(C₁₋₄ alkyl), CON(C₁₋₄ alkyl)₂, OC(O)R^{143,} thienyl, naphthyl and groups of the following formulae:

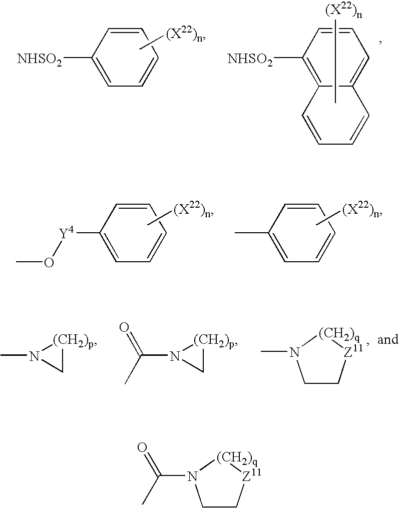

(c-2) C₁₋₂₂ alkyl or C₂₋₂₂ alkenyl, said alkyl or alkenyl being optionally substituted with five to forty-five halogen atoms,

(c-3) —Y⁵—C₃₋₇ cycloalkyl or —Y⁵—C₃₋₇ cycloalkenyl, said cycloalkyl or cycloalkenyl being optionally substituted with up to three substituent independently selected from:

(c-3-1) C₁₋₄ alkyl, hydroxy, OR¹⁴³, S(O)ₘR¹⁴³, amino, mono- or di-(C₁₋₄ alkyl)amino, CONH₂, CONH(C₁₋₄ alkyl) and CON(C₁₋₄ alkyl)₂, (c-4) phenyl or naphthyl, said phenyl or naphthyl being optionally substituted with up to seven (preferably up to seven) substituents independently selected from:

(c-4-1) halo, C₁₋₈ alkyl, C₁₋₄ alkyl-OH, hydroxy, C₁₋₈ alkoxy, halosubstituted C₁₋₈ alkyl, halosubstituted C₁₋₈ alkoxy, CN, nitro, S(O)ₘR¹⁴³, SO₂NH₂, SO₂NH(C₁₋₄ alkyl), SO₂N(C₁₋₄ alkyl)₂, amino, C₁₋₄ alkylamino, di-(C₁₋₄ alkyl)amino, CONH₂, CONH(C₁₋₄ alkyl), CON(C₁₋₄ alkyl)₂, OC(O)R¹⁴³, and phenyl optionally substituted with up to three substituents independently selected from halo, C₁₋₄ alkyl, hydroxy, OCH₃, CF₃, OCF₃, CN, nitro, amino, mono- or di-(C₁₋₄ alkyl)amino, CO₂H, CO₂ (C₁₋₄ alkyl) and CONH₂,

(c-5) a monocyclic aromatic group as defined in (d) and (e) above, said aromatic group being optionally substituted with up to three substituents independently selected from:

(c-5-1) halo, C₁₋₈ alkyl, C₁₋₄ alkyl-OH, hydroxy, C₁₋₈ alkoxy, CF₃, OCF₃, CN, nitro, S(O)ₘR¹⁴³, amino, mono- or di-(C₁₋₄ alkyl)amino, CONH₂, CONH(C₁₋₄ alkyl), CON(C₁₋₄ alkyl)₂, CO₂H and CO₂ (C₁₋₄ alkyl), and —Y-phenyl, said phenyl being optionally substituted with up to three substituents independently selected halogen, C₁₋₄ alkyl, hydroxy, C₁₋₄ alkoxy, CF₃, OCF₃, CN, nitro, S(O)ₘR¹⁴³, amino, mono- or di-(C₁₋₄ alkyl)amino, CO₂H, CO₂ (C₁₋₄ alkyl), CONH₂, CONH(C₁₋₄ alkyl) and CON(C₁₋₄ alkyl)₂,

(c-6) a group of the following formula:

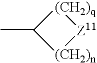

X²² is halo, C₁₋₄ alkyl, hydroxy, C₁₋₄ alkoxy, halosubstituted C₁₋₄ alkoxy, S(O)ₘ R¹⁴³, amino, mono- or di-(C₁₋₄ alkyl)amino, NHSO₂R¹⁴³, nitro, halosubstituted C₁₋₄ alkyl, CN, CO₂H, CO₂ (C₁₋₄ alkyl), C₁₋₄ alkyl-OH, C₁₋₄ alkylOR¹⁴³, CONH₂, CONH(C₁₋₄ alkyl) or CON(C₁₋₄ alkyl)₂;

R¹⁴³ is C₁₋₄ alkyl or halosubstituted C₁₋₄ alkyl;

m is 0, 1 or 2; n is 0, 1, 2 or 3; p is 1, 2, 3, 4 or 5; q is 2 or 3; Z¹¹ is oxygen, sulfur or NR¹⁴⁴; and

R¹⁴⁴ is hydrogen, C₁₋₆ alkyl, halosubstitutued C₁₋₄ alkyl or —Y⁵-phenyl, said phenyl being optionally substituted with up to two substituents independently selected from halo, C₁₋₄ alkyl, hydroxy, C₁₋₄ alkoxy, S(O)ₘR¹⁴³, amino, mono- or di-(C₁₋₄ alkyl)amino, CF₃, OCF₃, CN and nitro;

with the proviso that a group of formula —Y⁵-Q is not methyl or ethyl when X²² is hydrogen;

L⁴ is oxygen;

R¹⁴¹ is hydrogen; and

R¹⁴² is acetyl.

Materials that can serve as a cyclooxygenase-2 selective inhibitor of the present invention include aryl phenylhydrazides that are described in U.S. Pat. No. 6,077,869. Such aryl phenylhydrazides have the formula shown below in formula XXVIII:

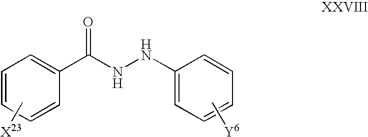

wherein:

X²³ and Y⁶ are selected from hydrogen, halogen, alkyl, nitro, amino or other oxygen and sulfur containing functional groups such as hydroxy, methoxy and methylsulfonyl.

Materials that can serve as a cyclooxygenase-2 selective inhibitor of the present invention include 2-aryloxy, 4-aryl furan-2-ones that are described in U.S. Pat. No. 6,140,515. Such 2-aryloxy, 4-aryl furan-2-ones have the formula shown below in formula XXIX:

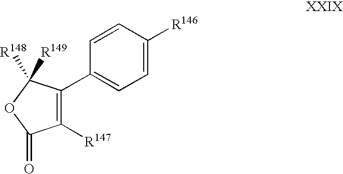

or a pharmaceutical salt thereof,

wherein:

R¹⁴⁶ is selected from the group consisting of SCH₃, —S(O)₂CH₃ and —S(O)₂NH₂;

R¹⁴⁷ is selected from the group consisting of OR¹⁵⁰, mono or di-substituted phenyl or pyridyl wherein the substituents are selected from the group consisting of methyl, chloro and F;

R¹⁵⁰ is unsubstituted or mono or di-substituted phenyl or pyridyl wherein the substituents are selected from the group consisting of methyl, chloro and F;

R¹⁴⁸ is H, C₁₋₄ alkyl optionally substituted with 1 to 3 groups of F, Cl or Br; and

R¹⁴⁹ is H, C₁₋₄ alkyl optionally substituted with 1 to 3 groups of F, Cl or Br, with the proviso that R¹⁴⁸ and R¹⁴⁹ are not the same.

Materials that can serve as a cyclooxygenase-2 selective inhibitor of the present invention include bisaryl compounds that are described in U.S. Pat. No. 5,994,379. Such bisaryl compounds have the formula shown below in formula XXX:

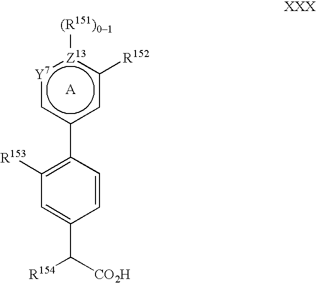

or a pharmaceutically acceptable salt, ester or tautomer thereof,

wherein:

Z¹³ is C or N;

when Z¹³ is N, R¹⁵¹ represents H or is absent, or is taken in conjunction with R¹⁵² as described below:

when Z¹³ is C, R¹⁵¹ represents H and R¹⁵² is a moiety which has the following characteristics:

(a) it is a linear chain of 3-4 atoms containing 0-2 double bonds, which can adopt an energetically stable transoid configuration and if a double bond is present, the bond is in the trans configuration,

(b) it is lipophilic except for the atom bonded directly to ring A, which is either lipophilic or non-lipophilic, and

(c) there exists an energetically stable configuration planar with ring A to within about 15 degrees;

or R¹⁵¹ and R¹⁵² are taken in combination and represent a 5- or 6-membered aromatic or non-aromatic ring D fused to ring A, said ring D containing 0-3 heteroatoms selected from O, S and N;

said ring D being lipophilic except for the atoms attached directly to ring A, which are lipophilic or non-lipophilic, and said ring D having available an energetically stable configuration planar with ring A to within about 15 degrees;

said ring D further being substituted with 1 R^{a} group selected from the group consisting of: C₁₋₂ alkyl, —OC₁₋₂ alkyl, —NHC₁₋₂ alkyl, —N(C₁₋₂ alkyl)₂, —C(O)C₁₋₂ alkyl, —S—C₁₋₂ alkyl and —C(S)C₁₋₂ alkyl;

Y⁷ represents N, CH or C—OC₁₋₃ alkyl, and when Z¹³ is N, Y⁷ can also represent a carbonyl group;

R¹⁵³ represents H, Br, Cl or F; and

R¹⁵⁴ represents H or CH₃.

Materials that can serve as a cyclooxygenase-2 selective inhibitor of the present invention include 1,5-diarylpyrazoles that are described in U.S. Pat. No. 6,028,202. Such 1,5-diarylpyrazoles have the formula shown below in formula XXXI:

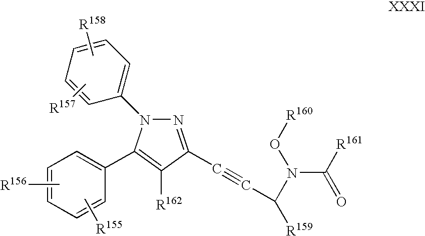

wherein:

R^{155,} R¹⁵⁶, R¹⁵⁷, and R¹⁵⁸ are independently selected from the groups consisting of hydrogen, C₁₋₅ alkyl, C₁₋₅ alkoxy, phenyl, halo, hydroxy, C₁₋₅ alkylsulfonyl, C₁₋₅ alkylthio, trihaloC₁₋₅ alkyl, amino, nitro and 2-quinolinylmethoxy;

R¹⁵⁹ is hydrogen, C₁₋₅ alkyl, trihaloC₁₋₅ alkyl, phenyl, substituted phenyl where the phenyl substitutents are halogen, C₁₋₅ alkoxy, trihaloC₁₋₅ alkyl or nitro or R¹⁵⁹ is heteroaryl of 5-7 ring members where at least one of the ring members is nitrogen, sulfur or oxygen;

R¹⁶⁰ is hydrogen, C₁₋₅ alkyl, phenyl C₁₋₅ alkyl, substituted phenyl C₁₋₅ alkyl where the phenyl substitutents are halogen, C₁₋₅ alkoxy, trihaloC₁₋₅ alkyl or nitro, or R¹⁶⁰ is C₁₋₅ alkoxycarbonyl, phenoxycarbonyl, substituted phenoxycarbonyl where the phenyl substitutents are halogen, C₁₋₅ alkoxy, trihaloC₁₋₅ alkyl or nitro;

R¹⁶¹ is C₁₋₁₀ alkyl, substituted C₁₋₁₀ alkyl where the substituents are halogen, trihaloC₁₋₅ alkyl, C₁₋₅ alkoxy, carboxy, C₁₋₅ alkoxycarbonyl, amino, C₁₋₅ alkylamino, diC₁₋₅ alkylamino, diC₁₋₅ alkylaminoC₁₋₅ alkylamino, C₁₋₅ alkylaminoC₁₋₅ alkylamino or a heterocycle containing 4-8 ring atoms where one more of the ring atoms is nitrogen, oxygen or sulfur, where said heterocycle may be optionally substituted with C₁₋₅ alkyl; or R¹⁶¹ is phenyl, substituted phenyl (where the phenyl substitutents are one or more of C₁₋₅ alkyl, halogen, C₁₋₅ alkoxy, trihaloC₁₋₅ alkyl or nitro), or R¹⁶¹ is heteroaryl having 5-7 ring atoms where one or more atoms are nitrogen, oxygen or sulfur, fused heteroaryl where one or more 5-7 membered aromatic rings are fused to the heteroaryl; or

R¹⁶¹ is NR¹⁶³ R¹⁶⁴ where R¹⁶³ and R¹⁶⁴ are independently selected from hydrogen and C₁₋₅ alkyl or R¹⁶³ and R¹⁶⁴ may be taken together with the depicted nitrogen to form a heteroaryl ring of 5-7 ring members where one or more of the ring members is nitrogen, sulfur or oxygen where said heteroaryl ring may be optionally substituted with C₁₋₅ alkyl;

R¹⁶² is hydrogen, C₁₋₅ alkyl, nitro, amino, and halogen;

and pharmaceutically acceptable salts thereof.

Materials that can serve as a cyclooxygenase-2 selective inhibitor of the present invention include 2-substituted imidazoles that are described in U.S. Pat. No. 6,040,320. Such 2-substituted imidazoles have the formula shown below in formula XXXII:

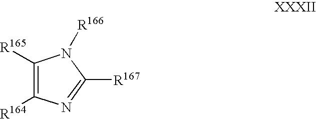

wherein:

R¹⁶⁴ is phenyl, heteroaryl wherein the heteroaryl contains 5 to 6 ring atoms, or

substituted phenyl;

wherein the substituents are independently selected from one or members of the group consisting of C₁₋₅ alkyl, halogen, nitro, trifluoromethyl and nitrile;

R¹⁶⁵ is phenyl, heteroaryl wherein the heteroaryl contains 5 to 6 ring atoms,

substituted heteroaryl;

wherein the substituents are independently selected from one or more members of the group consisting of C₁₋₅ alkyl and halogen, or

substituted phenyl,

wherein the substituents are independently selected from one or members of the group consisting of C₁₋₅ alkyl, halogen, nitro, trifluoromethyl and nitrile;

R¹⁶⁶ is hydrogen, SEM, C₁₋₅ alkoxycarbonyl, aryloxycarbonyl, arylC₁₋₅alkyloxycarbonyl, arylC₁₋₅ alkyl, phthalimidoC₁₋₅ alkyl, aminoC₁₋₅ alkyl, diaminoC₁₋₅ alkyl, succinimidoC₁₋₅ alkyl, C₁₋₅ alkylcarbonyl, arylcarbonyl, C₁₋₅ alkylcarbonylC₁₋₅ alkyl, aryloxycarbonylC₁₋₅ alkyl, heteroarylC₁₋₅ alkyl where the heteroaryl contains 5 to 6 ring atoms, or substituted arylC₁₋₅ alkyl,

wherein the aryl substituents are independently selected from one or more members of the group consisting of C₁₋₅ alkyl, C₁₋₅ alkoxy, halogen, amino, C₁₋₅ alkylamino, and diC₁₋₅ alkylamino;

R¹⁶⁷ is (A¹¹)ₙ-(CH¹⁶⁵)_{q}—X²⁴ wherein:

A¹¹ is sulfur or carbonyl;

n is 0 or 1;

q is 0-9;

X²⁴ is selected from the group consisting of hydrogen, hydroxy, halogen, vinyl, ethynyl, C₁₋₅ alkyl, C₃₋₇ cycloalkyl, C₁₋₅ alkoxy, phenoxy, phenyl, arylC₁₋₅ alkyl, amino, C₁₋₅ alkylamino, nitrile, phthalimido, amido, phenylcarbonyl, C₁₋₅ alkylaminocarbonyl, phenylaminocarbonyl, arylC₁₋₅ alkylaminocarbonyl, C₁₋₅ alkylthio, C₁₋₅ alkylsulfonyl, phenylsulfonyl,

substituted sulfonamido,

wherein the sulfonyl substituent is selected from the group consisting of C₁₋₅ alkyl, phenyl, araC_{1 5} alkyl, thienyl, furanyl, and naphthyl;

substituted vinyl,

wherein the substituents are independently selected from one or members of the group consisting of fluorine, bromine, chlorine and iodine,

substituted ethynyl,

wherein the substituents are independently selected from one or more members of the group consisting of fluorine, bromine chlorine and iodine,

substituted C₁₋₅ alkyl,

wherein the substituents are selected from the group consisting of one or more C₁₋₅ alkoxy, trihaloalkyl, phthalimido and amino,

substituted phenyl,

wherein the phenyl substituents are independently selected from one or more members of the group consisting of C₁₋₅ alkyl, halogen and C₁₋₅ alkoxy,

substituted phenoxy,

wherein the phenyl substituents are independently selected from one or more members of the group consisting of C₁₋₅ alkyl, halogen and C₁₋₅ alkoxy,

substituted C₁₋₅ alkoxy,

wherein the alkyl substituent is selected from the group consisting of phthalimido and amino,

substituted arylC₁₋₅ alkyl,

wherein the alkyl substituent is hydroxyl,

substituted arylC₁₋₅ alkyl,

wherein the phenyl substituents are independently selected from one or more members of the group consisting of C₁₋₅ alkyl, halogen and C₁₋₅ alkoxy,

substituted amido,

wherein the carbonyl substituent is selected from the group consisting of C₁₋₅ alkyl, phenyl, arylC₁₋₅ alkyl, thienyl, furanyl, and naphthyl, substituted phenylcarbonyl,

wherein the phenyl substituents are independently selected from one or members of the group consisting of C₁₋₅ alkyl, halogen and C₁₋₅ alkoxy,

substituted C₁₋₅ alkylthio,

wherein the alkyl substituent is selected from the group consisting of hydroxy and phthalimido,

substituted C₁₋₅ alkylsulfonyl,

wherein the alkyl substituent is selected from the group consisting of hydroxy and phthalimido,

substituted phenylsulfonyl,

wherein the phenyl substituents are independently selected from one or members of the group consisting of bromine, fluorine, chlorine, C₁₋₅ alkoxy and trifluoromethyl,

with the proviso:

if A¹¹ is sulfur and X²⁴ is other than hydrogen, C₁₋₅ alkylaminocarbonyl, phenylaminocarbonyl, arylC₁₋₅ alkylaminocarbonyl, C₁₋₅ alkylsulfonyl or phenylsulfonyl, then q must be equal to or greater than 1;

if A¹¹ is sulfur and q is 1, then X²⁴ cannot be C₁₋₂ alkyl;

if A¹¹ is carbonyl and q is 0, then X²⁴ cannot be vinyl, ethynyl, C₁₋₅ alkylaminocarbonyl, phenylaminocarbonyl, arylC₁₋₅ alkylaminocarbonyl,C₁₋₅ alkylsulfonyl or phenylsulfonyl;

if A¹¹ is carbonyl, q is 0 and X²⁴ is H, then R¹⁶⁶ is not SEM (2-(trimethylsilyl)ethoxymethyl);

if n is 0 and q is 0, then X²⁴ cannot be hydrogen;

and pharmaceutically acceptable salts thereof.

Materials that can serve as a cyclooxygenase-2 selective inhibitor of the present invention include 1,3- and 2,3-diarylcycloalkano and cycloalkeno pyrazoles that are described in U.S. Pat. No. 6,083,969. Such 1,3- and 2,3-diarylpyrazole compounds have the general formulas shown below in formulas XXXIII and XXXIV:

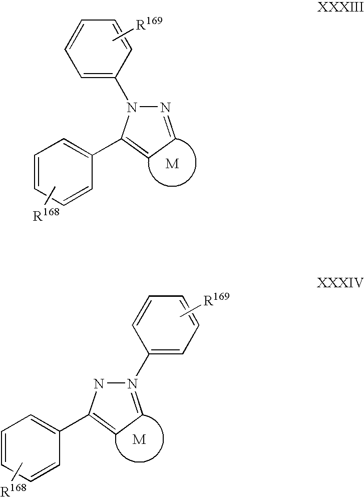

wherein:

R¹⁶⁸ and R¹⁶⁹ are independently selected from the group consisting of hydrogen, halogen, (C₁-C₆)alkyl, (C₁-C₆)alkoxy, nitro, amino, hydroxy, trifluoro, —S(C₁-C₆)alkyl, —SO(C₁-C₆)alkyl and —SO₂(C₁-C₆)alkyl; and the fused moiety M is a group selected from the group consisting of an optionally substituted cyclohexyl and cycloheptyl group having the formulae:

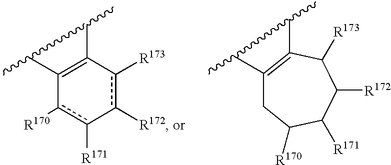

wherein:

R¹⁷⁰ is selected from the group consisting of hydrogen, halogen, hydroxy and carbonyl;

or R¹⁷⁰ and R¹⁷¹ taken together form a moiety selected from the group consisting of —OCOCH₂—, —ONH(CH₃)COCH₂—, —OCOCH.dbd. and —O—;

R¹⁷¹ and R¹⁷² are independently selected from the group consisting of hydrogen, halogen, hydroxy, carbonyl, amino, (C₁-C₆)alkyl, (C₁-C₆)alkoxy, ═NOH, —NR¹⁷⁴R¹⁷⁵, —OCH₃, —OCH₂CH₃, —OSO₂NHCO₂CH₃, ═CHCO₂CH₂CH₃, —CH₂CO_{2 l H, —CH}₂CO₂CH₃, —CH₂CO₂CH₂ CH₃, —CH₂CON(CH₃)₂, —CH₂CO₂NHCH₃, —CHCHCO₂CH₂CH₃, —OCON(CH₃)OH, —C(COCH₃)₂, di(C₁-C₆)alkyl and di(C₁-C₆)alkoxy;

R¹⁷³ is selected from the group consisting of hydrogen, halogen, hydroxy, carbonyl, amino, (C₁-C₆)alkyl, (C₁-C₆)alkoxy and optionally substituted carboxyphenyl, wherein substituents on the carboxyphenyl group are selected from the group consisting of halogen, hydroxy, amino, (C₁-C₆)alkyl and (C₁-C₆)alkoxy;

or R¹⁷² and R¹⁷³ taken together form a moiety selected from the group consisting of —O— and

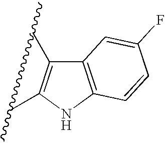

R¹⁷⁴ is selected from the group consisting of hydrogen, OH, —OCOCH₃, —COCH₃ and (C₁-C₆)alkyl; and

R¹⁷⁵ is selected from the group consisting of hydrogen, OH, —OCOCH₃, —COCH₃, (C₁-C₆)alkyl, —CONH₂ and —SO₂CH₃; with the proviso that

if M is a cyclohexyl group, then R¹⁷⁰ through R¹⁷³ may not all be hydrogen; and

pharmaceutically acceptable salts, esters and pro-drug forms thereof.

Materials that can serve as a cyclooxygenase-2 selective inhibitor of the present invention include esters derived from indolealkanols and novel amides derived from indolealkylamides that are described in U.S. Pat. No. 6,306,890. Such compounds have the general formula shown below in formula XXXV:

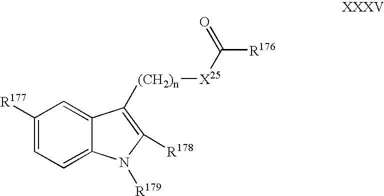

wherein:

R¹⁷⁶ is C₁ to C₆ alkyl, C₁ to C₆ branched alkyl, C₄ to C₈ cycloalkyl, C₁ to C₆ hydroxyalkyl, branched C₁ to C₆ hydroxyalkyl, hydroxy substituted C₄ to C₈ aryl, primary, secondary or tertiary C₁ to C₆ alkylamino, primary, secondary or tertiary branched C₁ to C₆ alkylamino, primary, secondary or tertiary C₄ to C₈ arylamino, C₁ to C₆ alkylcarboxylic acid, branched C₁ to C₆ alkylcarboxylic acid, C₁ to C₆ alkylester, branched C₁ to C₆ alkylester, C₄ to C₈ aryl, C₄ to C₈ arylcarboxylic acid, C₄ to C₈ arylester, C₄ to C₈ aryl substituted C₁ to C₆ alkyl, C₄ to C₈ heterocyclic alkyl or aryl with O, N or S in the ring, alkyl-substituted or aryl-substituted C₄ to C₈ heterocyclic alkyl or aryl with O, N or S in the ring, or halo-substituted versions thereof, where halo is chloro, bromo, fluoro or iodo;

R¹⁷⁷ is C₁ to C₆ alkyl, C₁ to C₆ branched alkyl, C₄ to C₈ cycloalkyl, C₄ to C₈ aryl, C₄ to C₈ aryl-substituted C₁ to C₆ alkyl, C, to C₆ alkoxy, C₁ to C₆ branched alkoxy, C₄ to C₈ aryloxy, or halo-substituted versions thereof or R¹⁷⁷ is halo where halo is chloro, fluoro, bromo, or iodo;

R¹⁷⁸ is hydrogen, C₁ to C₆ alkyl or C₁ to C₆ branched alkyl;

R¹⁷⁹ is C₁ to C₆ alkyl, C₄ to C₈ aroyl, C₄ to C₈ aryl, C₄ to C₈ heterocyclic alkyl or aryl with O, N or S in the ring, C₄ to C₈ aryl-substituted C₁ to C₆ alkyl, alkyl-substituted or aryl-substituted C₄ to C₈ heterocyclic alkyl or aryl with O, N or S in the ring, alkyl-substituted C₄ to C₈ aroyl, or alkyl-substituted C₄ to C₈ aryl, or halo-substituted versions thereof where halo is chloro, bromo, or iodo;

n is 1, 2, 3, or 4; and

X²⁵ is O, NH, or N—R¹⁸⁰, where R¹⁸⁰ is C₁ to C₆ alkyl or C₁ to C₆ branched alkyl.

Materials that can serve as a cyclooxygenase-2 selective inhibitor of the present invention include pyridazinone compounds that are described in U.S. Pat. No. 6,307,047. Such pyridazinone compounds have the formula shown below in formula XXXVI:

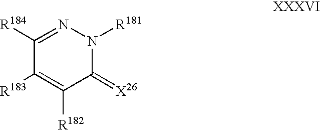

or a pharmaceutically acceptable salt, ester, or prodrug thereof,

wherein:

X²⁶ is selected from the group consisting of O, S, —NR¹⁸⁵, —NOR^{a}, and —NNR^{b}R^{c};

R¹⁸⁵ is selected from the group consisting of alkenyl, alkyl, aryl, arylalkyl, cycloalkenyl, cycloalkenylalkyl, cycloalkyl, cycloalkylalkyl, heterocyclic, and heterocyclic alkyl;

R^{a}, R^{b}, and R^{c} are independently selected from the group consisting of alkyl, aryl, arylalkyl, cycloalkyl, and cycloalkylalkyl;

R¹⁸¹ is selected from the group consisting of alkenyl, alkoxy, alkoxyalkyl, alkoxyiminoalkoxy, alkyl, alkylcarbonylalkyl, alkylsulfonylalkyl, alkynyl, aryl, arylalkenyl, arylalkoxy, arylalkyl, arylalkynyl, arylhaloalkyl, arylhydroxyalkyl, aryloxy, aryloxyhaloalkyl, aryloxyhydroxyalkyl, arylcarbonylalkyl, carboxyalkyl, cyanoalkyl, cycloalkenyl, cycloalkenylalkyl, cycloalkyl, cycloalkylalkyl, cycloalkylidenealkyl, haloalkenyl, haloalkoxyhydroxyalkyl, haloalkyl, haloalkynyl, heterocyclic, heterocyclic alkoxy, heterocyclic alkyl, heterocyclic oxy, hydroxyalkyl, hydroxyiminoalkoxy, —(CH₂)ₙ C(O)R¹⁸⁶, —(CH₂)ₙCH(OH)R¹⁸⁶, —(CH₂)ₙC(NOR^{d})R¹⁸⁶, —(CH₂), CH(NOR^{d})R¹⁸⁶, —(CH₂)ₙCH(NR^{d}R^{e})R¹⁸⁶, —R¹⁸⁷ R¹⁸⁸, —(CH₂)ₙ C≡CR¹⁸⁸, —(CH₂)ₙ[CH(CX^{26′}₃)]ₘ (CH₂)ₚ R¹⁸⁸, —(CH₂)ₙ (CX^{26′}₂)ₘ (CH₂)ₚ R^{188,} and —(CH₂)ₙ (CHX^{26′})ₘ (CH₂)ₘ R¹⁸⁸;

R¹⁸⁶ is selected from the group consisting of hydrogen, alkenyl, alkyl, alkynyl, aryl, arylalkyl, cycloalkenyl, cycloalkyl, haloalkenyl, haloalkyl, haloalkynyl, heterocyclic, and heterocyclic alkyl;

R¹⁸⁷ is selected from the group consisting of alkenylene, alkylene, halo-substituted alkenylene, and halo-substituted alkylene;

R¹⁸⁸ is selected from the group consisting of hydrogen, alkenyl, alkyl, alkynyl, aryl, arylalkyl, cycloalkyl, cycloalkenyl, haloalkyl, heterocyclic, and heterocyclic alkyl;

R^{d} and R^{e} are independently selected from the group consisting of hydrogen, alkenyl, alkyl, alkynyl, aryl, arylalkyl, cycloalkenyl, cycloalkyl, haloalkyl, heterocyclic, and heterocyclic alkyl;

X^{26′} is halogen;

m is an integer from 0-5;

n is an integer from 0-10; and

p is an integer from 0-10; and

R¹⁸², R¹⁸³, and R¹⁸⁴ are independently selected from the group consisting of hydrogen, alkenyl, alkoxyalkyl, alkoxyiminoalkoxy, alkoxyiminoalkyl, alkyl, alkynyl, alkylcarbonylalkoxy, alkylcarbonylamino, alkylcarbonylaminoalkyl, aminoalkoxy, aminoalkylcarbonyloxyalkoxy aminocarbonylalkyl, aryl, arylalkenyl, arylalkyl, arylalkynyl, carboxyalkylcarbonyloxyalkoxy, cyano, cycloalkenyl, cycloalkyl, cycloalkylidenealkyl, haloalkenyloxy, haloalkoxy, haloalkyl, halogen, heterocyclic, hydroxyalkoxy, hydroxyiminoalkoxy, hydroxyiminoalkyl, mercaptoalkoxy, nitro, phosphonatoalkoxy, Y⁸, and Z¹⁴;

provided that one of R¹⁸², R¹⁸³, or R¹⁸⁴ must be Z¹⁴, and further provided that only one of R¹⁸², R¹⁸³, or R¹⁸⁴ is Z¹⁴;

Z¹⁴ is selected from the group consisting of:

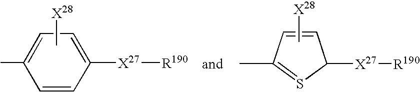

²⁷ is selected from the group consisting of S(O)₂, S(O)(NR¹⁹¹), S(O), Se(O)₂, P(O)(OR¹⁹²), and P(O)(NR¹⁹³ R¹⁹⁴);

X²⁸ is selected from the group consisting of hydrogen, alkenyl, alkyl, alkynyl and halogen;

R¹⁹⁰ is selected from the group consisting of alkenyl, alkoxy, alkyl, alkylamino, alkylcarbonylamino, alkynyl, amino, cycloalkenyl, cycloalkyl, dialkylamino, —NHNH₂, and —NCHN(R¹⁹¹)R¹⁹²;

R¹⁹¹, R¹⁹², R¹⁹³, and R¹⁹⁴ are independently selected from the group consisting of hydrogen, alkyl, and cycloalkyl, or R¹⁹³ and R¹⁹⁴ can be taken together, with the nitrogen to which they are attached, to form a 3-6 membered ring containing 1 or 2 heteroatoms selected from the group consisting of O, S, and NR¹⁸⁸;

Y⁸ is selected from the group consisting of —OR¹⁹⁵, —SR¹⁹⁵, —C(R¹⁹⁷)(R¹⁹⁸)R¹⁹⁵, —C(O)R¹⁹⁵, —C(O)OR¹⁹⁵, —N(R¹⁹⁷)C(O)R¹⁹⁵, —NC(R¹⁹⁷)R¹⁹⁵, and —N(R¹⁹⁷)R¹⁹⁵;

R¹⁹⁵ is selected from the group consisting of hydrogen, alkenyl, alkoxyalkyl, alkyl, alkylthioalkyl, alkynyl, cycloalkenyl, cycloalkenylalkyl, cycloalkyl, cycloalkylalkyl, aryl, arylalkyl, heterocyclic, heterocyclic alkyl, hydroxyalkyl, and NR¹⁹⁹ R²⁰⁰; and

R¹⁹⁷, R¹⁹⁸, R¹⁹⁹, and R²⁰⁰ are independently selected from the group consisting of hydrogen, alkenyl, alkoxy, alkyl, cycloalkenyl, cycloalkyl, aryl, arylalkyl, heterocyclic, and heterocyclic alkyl.

Materials that can serve as a cyclooxygenase-2 selective inhibitor of the present invention include benzosulphonamide derivatives that are described in U.S. Pat. No. 6,004,948. Such benzosulphonamide derivatives have the formula shown below in formula XXXVII:

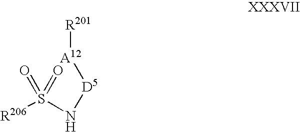

herein:

A¹² denotes oxygen, sulphur or NH;

R²⁰¹ denotes a cycloalkyl, aryl or heteroaryl group optionally mono- or polysubstituted by halogen, alkyl, CF₃ or alkoxy;

D⁵ denotes a group of formula XXXVIII or XXXIX:

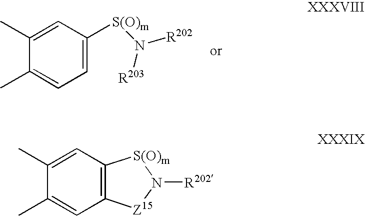

R²⁰² and R²⁹³ independently of each other denote hydrogen, an optionally polyfluorinated alkyl radical, an aralkyl, aryl or heteroaryl radical or a radical (CH₂)ₙ—X²⁹; or

R²⁰² and R²⁰³ together with the N-atom denote a three- to seven-membered, saturated, partially or totally unsaturated heterocycle with one or more heteroatoms N, O, or S, which may optionally be substituted by oxo, an alkyl, alkylaryl or aryl group or a group (CH₂)ₙ—X²⁹, R^{202′} denotes hydrogen, an optionally polyfluorinated alkyl group, an aralkyl, aryl or heteroaryl group or a group (CH₂)ₙ—X²⁹,

wherein:

X²⁹ denotes halogen, NO₂, —OR²⁰⁴, —COR²⁰⁴, —CO₂R²⁰⁴, —OCO₂R²⁰⁴, —CN, —CONR²⁰⁴OR²⁰⁵, —CONR²⁰⁴R²⁰⁵, —SR²⁰⁴, —S(O)R²⁰⁴, —S(O)₂R²⁰⁴, —NR²⁰⁴R²⁰⁵, —NHC(O)R²⁰⁴, —NHS(O)₂R²⁰⁴; Z¹⁵ denotes —CH₂—, —CH₂—CH₂—, —CH₂—CH₂—CH₂—, —CH₂—CH═CH—, —CH═CH—CH₂—, —CH₂—CO—, —CO—CH₂—, —NHCO—, —CONH—, —NHCH₂—, —CH₂NH—, —N═CH—, —NHCH—, —CH₂—CH₂—NH—, —CH═CH—, >N—R²⁰³, >C═O, >S(O)ₘ;

R²⁰⁴ and R²⁰⁵ independently of each other denote hydrogen, alkyl, aralkyl or aryl;

n is an integer from 0 to 6;

R²⁰⁶ is a straight-chained or branched C₁₋₄ -alkyl group which may optionally be mono- or polysubstituted by halogen or alkoxy, or R²⁰⁶ denotes CF₃; and

m denotes an integer from 0 to 2;

with the proviso that A¹² does not represent O if R²⁰⁶ denotes CF₃;

and the pharmaceutically acceptable salts thereof.

Cox-2 selective inhibitors that are useful in the subject method and compositions can include the compounds that are described in U.S. Pat. Nos. 6,169,188, 6,020,343, 5,981,576 ((methylsulfonyl)phenyl furanones); U.S. Pat. No. 6,222,048 (diaryl-2-(5H)-furanones); U.S. Pat. No. 6,057,319 (3,4-diaryl-2-hydroxy-2,5-dihydrofurans); U.S. Pat. No. 6,046,236 (carbocyclic sulfonamides); U.S. Pat. Nos. 6,002,014 and 5,945,539 (oxazole derivatives); and U.S. Pat. No. 6,359,182 (C-nitroso compounds).

Cyclooxygenase-2 selective inhibitors that are useful in the present invention can be supplied by any source as long as the cyclooxygenase-2-selective inhibitor is pharmaceutically acceptable. Cyclooxygenase-2-selective inhibitors can be isolated and purified from natural sources or can be synthesized. Cyclooxygenase-2-selective inhibitors should be of a quality and purity that is conventional in the trade for use in pharmaceutical products.

In an embodiment of the present method, the Cox-2 inhibitor is administered in combination with a glucocorticoid. Glucocorticoids that are useful in the present invention are steroid hormones that are produced by the adrenal cortex that help the body of a subject respond to stress and fatigue by increasing metabolism and inhibiting the inflammatory response. Examples of useful glucocorticoids include mometasone, fluticasone-17-proprionate, budesonide, beclomethasone, betamethasone, methyl-prednisolone, dexamethasone, prednisolone, hydrocortisone (cortisol), triamcinolone, cortisone, corticosterone and prednisone. Each of these glucocorticoids can be supplied in the form of a salt, or prodrug, if desirable. Also included in the meaning of glucocorticoids in the present invention, are non-steroidal GC mimics that are not dissociated, and steroidal and non-steroidal GC analogs and mimics, respectively, that are dissociated. When the term “dissociated” is used herein to describe glucocorticoid analogs and mimics, what is meant are steroidal or non-steroidal glucocorticoid analogs or mimics, respectively, that retain anti-inflammatory/immunosuppressive efficacy, but manifest the reduction of one or multiple side effects.

Hydrocortisone, also known as cortisol, is a steroid with glucocorticoid activity and some mineralocorticoid effects. In addition to all conventional uses for a glucocorticoid, it is indicated for septic shock, adrenal insufficiency, congenital adrenal hyperplasia and allergic reaction, and is available as hydrocortisone, hydrocortisone acetate, hydrocortisone butyrate, hydrocortisone cypionate, hydrocortisone probutate, hydrocortisone sodium phosphate, hydrocortisone sodium succinate, and hydrocortisone valerate. Dose, depending on disease, is 20-240 mg/day.

Beclomethasone is available as beclomethasone dipropionate. In addition to all conventional uses for a glucocorticoid, it is used for rhinitis, to prevent recurrence of nasal polyps following surgical removal, and for bronchial asthma. Dosage for adults and children over 12 years old, administered by inhalation, is from 84 micrograms/day to 840 micrograms/day.

Cortisone is available as cortisone acetate. In addition to all conventional uses for a glucocorticoid, it is used in replacement therapy in chronic cortical insufficiency, and on a short-term for inflammatory or allergic disorders. Dosage for initial treatment, or during crisis, is from 25 to 300 mg/day; as an inflammatory is 25-1-50 mg/day; and for acute rheumatic fever is 200 mg/day. Maintenance dose is 0.5 to 0.75 mg/kg/day.

Dexamethasone is available as dexamethasone, dexamethasone sodium phosphate, and dexamethasone acetate. In addition to all conventional uses for a glucocorticoid, it is used for acute allergic disorders, to test for adrenal cortical hyperfunction, cerebral edema due to brain tumor, craniotomy, or head injury. Dosage is initially 0.75-9 mg/day, gradually reduced to a maintenance dose of 0.5-3 mg/day.

Methylprednisolone is available as methylprednisolone, methylprednisolone acetate, and methylprednisolone sodium succinate. In addition to all conventional uses for a glucocorticoid, it is used for rheumatoid arthritis, severe hepatitis due to alcoholism, within 8 hr of severe spinal cord injury (to improve neurologic function), and for septic shock. Dosage for adults for rheumatoid arthritis is 6-16 mg/day, decreased gradually; for acute indications is 20-96 mg/day, decreasing to a maintenance dosage of 8-20 mg/day.

Betamethasone is available as betamethasone, betamethasone dipropionate, betamethasone sodium phosphate, betamethasone acetate, and betamethasone valerate. In addition to all conventional uses for a glucocorticoid, it is used for prevention of respiratory distress syndrome is premature infants. Dosage is from 0.5 to 9 mg/day, with dosages adjusted downward to maintenance level.

Glucocorticoids that are useful in the present invention can be of any purity or grade, as long as the preparation is of a quality suitable for pharmaceutical use. The glucocorticoid can be provided in pure form, or it can be accompanied with impurities or commonly associated compounds that do not affect its physiological activity or safety. The glucocorticoid can be supplied as a pure compound, or in the form of a pharmaceutically active salt. The glucocorticoid can be supplied in the form of a prodrug, an isomer, a racemic mixture, or in any other chemical form or combination that, under physiological conditions, provides the glucocorticoid.

The term “subject”, for purposes of treatment, includes any vertebrate. The subject is typically a mammal. “Mammal”, as that term is used herein, refers to any animal classified as a mammal, including humans, domestic and farm animals, and zoo, sports, or pet animals, such as dogs, horses, cats, cattle, etc. Preferably, the mammal is a human.

Diseases and disorders that are amenable to prevention or treatment by the present methods and compositions are “T cell mediated inflammatory/autoimmune diseases and disorders”. As those terms are used herein, they should be understood to mean those diseases or disorders that are associated with T cell-mediated inflammatory processes or T cell mediated autoimmune processes. In particular, the present invention is useful for diseases or disorders that are mediated by activated circulatory T cells that are present in the blood, the spleen and the lymph nodes. The benefits of the present invention are particularly useful in those subjects having a deficiency of glucocorticoid regulation of immune response.

T cells can be activated by contact with a T cell activating agent. Such agents include exogenous or endogenous T cell activating antigens (attached to suitable presenter cells), or can be T cell-specific antibodies, such as a CD3ε antibody.

T cell-mediated inflammatory/autoimmune diseases and disorders have been discussed above, and examples include, without limitation, graft vs. host disease, toxic shock syndrome, bacterial sepsis, viral sepsis, food poisoning (superantigen mediated), transplant rejection, immunosuppression using anti-CD3 antibodies or other compounds (OKT-3, etc), multiple sclerosis, systemic lupus erythematosus, rheumatoid arthritis, and inflammatory bowel disease.

As used herein, the terms “subject in need of such treatment” refer to a subject having some type of glucocorticoid regulation deficiency, where the subject is suffering from, or at risk of suffering from, symptoms associated with a T cell mediated inflammatory/autoimmune disease and/or disorder. In some embodiments of the invention, the subject in need of such treatment is one that is already receiving treatment with glucocorticoids.

When it is said that the subject has a “glucocorticoid regulation deficiency”, it is meant that the subject has a glucocorticoid resistance, a glucocorticoid insufficiency, or has experienced a T cell activating stimulus of a strength sufficient to overwhelm the subject's endogenous glucocorticoid regulation of the production of inflammation and immune-related compounds, such as eicosanoids, cytokines, and associated enzymes and other compounds.

Clinical syndromes of glucocorticoid resistance can be familial or acquired, and can be generalized or tissue-specific. Examples of generalized glucocorticoid resistance include generalized inherited glucocorticoid resistance (GIGR), and acquired generalized glucocorticoid resistance, which can occur in a subgroup of patients with acquired immunodeficiency syndrome (AIDS). Subjects could show a glucocorticoid resistance on account of an abnormal GRα/GRβ ratio, or due to resistance developed in response to either chronic inflammatory stimuli or chronic GC treatment. Glucocorticoid resistance can be iatrogenic.

Subjects can show glucocorticoid insufficiency on account of primary or chronic adrenocortical insufficiency (Addison's disease), or autoimmune processes.

Glucocorticoid regulation deficiency also includes cases where subjects having an otherwise normally functioning T cell-mediated immune response are challenged with a T cell activating stimulus, such as are present in toxic shock, a graft vs. host response, immune response triggered by trauma, or infectious disease, that is sufficiently strong that it overwhelms the GC/GR regulatory system and causes hyper-production of Cox-2.

In an embodiment of the present method, a subject having a glucocorticoid regulation deficiency can be prevented from experiencing, or treated for the symptoms of, T cell mediated inflammatory/autoimmune diseases and disorders. The method comprises administering to a subject in need of such prevention or treatment an effective amount of a cyclooxygenase-2 inhibitor or prodrug thereof. The Cox-2 inhibitor can be administered to the subject alone, or in combination with a glucocorticoid. In preferred embodiments, the effective amount constitutes a therapeutically effective amount. For methods of prevention, the subject is any human or animal subject, and preferably is a subject that is in need of prevention and/or treatment of a T cell mediated inflammatory/autoimmune disease or disorder. The subject may be at risk due to genetic predisposition, sedentary lifestyle, diet, exposure to disorder-causing agents, exposure to traumatic event, exposure to pathogenic agents and the like.

In another embodiment of the present method, the subject is treated with a cyclooxygenase-2 inhibitor or prodrug thereof and a glucocorticoid. In one embodiment, the subject is treated with an amount of a Cox-2 inhibitor and an amount of a glucocorticoid, where the amount of the Cox-2 inhibitor and the amount of the glucocorticoid together provide a dosage or amount of the combination that is sufficient to constitute an effective amount of the combination. In preferred embodiments, the effective amount is a therapeutically effective amount.

As used herein, an “effective amount” means the dose or effective amount to be administered to a patient and the frequency of administration to the subject which is readily determined by one of ordinary skill in the art, by the use of known techniques and by observing results obtained under analogous circumstances. The dose or effective amount to be administered to a patient and the frequency of administration to the subject can be readily determined by one of ordinary skill in the art by the use of known techniques and by observing results obtained under analogous circumstances. In determining the effective amount or dose, a number of factors are considered by the attending diagnostician, including, but not limited to, the potency and duration of action of the compounds used, the nature and severity of the illness to be treated, as well as on the sex, age, weight, general health and individual responsiveness of the patient to be treated, and other relevant circumstances.

The phrase “therapeutically-effective” indicates the capability of an agent to prevent, or mitigate the severity of, the disorder, while avoiding adverse side effects typically associated with alternative therapies. The phrase “therapeutically-effective” is intended to qualify the amount of one or more agents for use in the therapy which will achieve the goal of improvement in the severity of symptoms associated with T cell mediated inflammatory/autoimmune diseases or disorders, while limiting adverse side effects typically associated with alternative therapies.

Those skilled in the art will appreciate that dosages may also be determined with guidance from Goodman & Goldman's The Pharmacological Basis of Therapeutics, Ninth Edition (1996), Appendix II, pp. 1707-1711.

The amount of Cox-2 inhibitor that is used in the subject method may be an amount that is sufficient to constitute an effective amount. Preferably, such amount would be a therapeutically effective amount. The therapeutically effective amount can also be considered to be a maximally saturating amount, or, alternatively, as the maximum amount that can be administered while avoiding the incidence of gastrointestinal ulcers caused by crossover Cox-1 inhibition.

In an embodiment of the present method where the Cox-2 inhibitor is a Cox-2 selective inhibitor, the amount of Cox-2 selective inhibitor that is used in the novel method of treatment preferably ranges from about 0.01 to about 100 milligrams per day per kilogram of body weight of the subject (mg/day·kg), more preferably from about 0.1 to about 50 mg/day-kg, even more preferably from about 1 to about 20 mg/day·kg.

When the Cox-2 selective inhibitor comprises rofecoxib, it is preferred that the amount used is within a range of from about 0.15 to about 1.0 mg/day kg, and even more preferably from about 0.18 to about 0.4 mg/day kg.

When the Cox-2 selective inhibitor comprises etoricoxib, it is preferred that the amount used is within a range of from about 0.5 to about 5 mg/day·kg, and even more preferably from about 0.8 to about 4 mg/day·kg.

When the Cox-2 selective inhibitor comprises celecoxib, it is preferred that the amount used is within a range of from about 1 to about 10 mg/day·kg, even more preferably from about 1.4 to about 8.6 mg/day·kg, and yet more preferably from about 2 to about 3 mg/day·kg.

When the Cox-2 selective inhibitor comprises valdecoxib or parecoxib sodium, it is preferred that the amount used is within a range of from about 0.1 to about 3 mg/day·kg, and even more preferably from about 0.3 to about 1 mg/day·kg.

In those embodiments of the present invention where a glucocorticoid is administered in combination with the cyclooxygenase-2 inhibitor, the amount of the glucocorticoid that is administered is an effective amount. The amount of a glucocorticoid that constitutes an effective amount depends upon the type of glucocorticoid that is used and the route of administration. The effective amount for commercially available glucocorticoid preparations is provided in the prescribing information that is available from the manufacturers and suppliers of the particular glucocorticoid of interest. By way of example, equivalent dosages (expressed in milligrams) have been determined for betamethasone (0.6-0.75 mg), dexamethasone (0.75 mg), hydrocortisone (20), methylprednisolone (4), prednisolone (5), and prednisone (5). See Am. Soc. of Health System Pharmacists, Dexamethasone Sodium Phosphate for Injection, at http://www.ashp.org/shortage/dexamethasome. html, on May 08, 2002.

The frequency of dose will depend upon the half-life of the cyclooxygenase-2 inhibitor. If the Cox-2 inhibitor has a short half-life (e.g. from about 2 to 10 hours) it may be necessary to give one or more doses per day. Alternatively, if the Cox-2 inhibitor has a long half-life (e.g. from about 2 to about 15 days) it may only be necessary to give a dosage once per day, per week, or even once every 1 or 2 months. A preferred dosage rate is to administer the dosage amounts described above to a subject once per day.

For the purposes of calculating and expressing a dosage rate, all dosages that are expressed herein are calculated on an average amount-per-day basis irrespective of the dosage rate. For example, one 100 mg dosage of an ingredient taken once every two days would be expressed as a dosage rate of 50 mg/day. Similarly, the dosage rate of an ingredient where 50 mg is taken twice per day would be expressed as a dosage rate of 100 mg/day.

For the purposes of calculation of a dosage rate for the present method, the weight of an adult human is assumed to be 70 kg.

In the present method, and in the subject compositions, the Cox-2 inhibitor may be administered alone, or in combination with a glucocorticoid. When the Cox-2 inhibitor is a Cox-2 selective inhibitor, it is preferred that the weight ratio of the amount of the amount of Cox-2 selective inhibitor to the amount of the glucocorticoid that is administered to the subject is within a range of from about 0.03:1 to about 35,000:1, more preferred is a range of from about 0.3:1 to about 14,000:1, even more preferred is a range of from about 0.5:1 to about 100:1.

The combination of a Cox-2 inhibitor and a glucocorticoid can be supplied in the form of a novel therapeutic composition that is believed to be within the scope of the present invention. The relative amounts of each component in the therapeutic composition may be varied and may be as described just above. The Cox-2 inhibitor and the glucocorticoid that are described above can be provided in the therapeutic composition so that the preferred amounts of each of the components are supplied by a single dosage, a single injection or a single capsule for example, or, by up to four, or more, single dosage forms.

When the novel combination is supplied along with a pharmaceutically acceptable carrier, a pharmaceutical composition is formed. A pharmaceutical composition of the present invention is directed to a composition suitable for the prevention or treatment of T cell mediated inflammatory/autoimmune diseases and disorders.

The pharmaceutical composition comprises a pharmaceutically acceptable carrier, a cyclooxygenase-2 inhibitor and a glucocorticoid.

Pharmaceutically acceptable carriers include, but are not limited to, physiological saline, Ringer's solution, phosphate solution or buffer, buffered saline and other carriers known in the art. Pharmaceutical compositions may also include stabilizers, anti-oxidants, colorants, and diluents. Pharmaceutically acceptable carriers and additives are chosen such that side effects from the pharmaceutical compound(s) are minimized and the performance of the compound(s) is not canceled or inhibited to such an extent that treatment is ineffective.

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxaloacetic acid, fumaric acid, propionic acid, aspartic acid, glutamic acid, benzoic acid, and the like.

Also included in the combination of the invention are the isomeric forms and tautomers and the pharmaceutically acceptable salts of cyclooxygenase-2 inhibitors and glucocorticoids. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

Suitable pharmaceutically acceptable base addition salts of compounds of the present invention include metallic ion salts and organic ion salts. More preferred metallic ion salts include, but are not limited to, appropriate alkali metal (group Ia) salts, alkaline earth metal (group IIa) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The terms “treating” or “to treat,” mean to alleviate signs and symptoms, eliminate the causation either on a temporary or permanent basis, prevent or slow the appearance of symptoms, or to retard or halt disease progression. The term “treatment” includes alleviation, elimination of causation of, or prevention of symptoms associated with, but not limited to, any of the diseases or disorders described herein, and also retarding or halting (of disease progression for these diseases or disorders.

The subject pharmaceutical compositions may be administered enterally and parenterally. Parenteral administration includes subcutaneous, intramuscular, intradermal, intramammary, intravenous, and other administrative methods known in the art. Enteral administration includes solution, tablets, sustained release capsules, enteric coated capsules, and syrups. When administered, the pharmaceutical composition may be at or near body temperature.

The phrases “combination therapy”, “co-administration”, “administration with”, or “co-therapy”, in defining the use of a cyclooxygenase-2 inhibitor agent and a glucocorticoid agent, is intended to embrace administration of each agent in a sequential manner in a regimen that will provide beneficial effects of the drug combination, and is intended as well to embrace co-administration of these agents in a substantially simultaneous manner, such as in a single capsule or dosage device having a fixed ratio of these active agents or in multiple, separate capsules or dosage devices for each agent, where the separate capsules or dosage devices can be taken together contemporaneously, or taken within a period of time sufficient to receive a beneficial effect from both of the constituent agents of the combination.

Although the combination of the present invention may include administration of a cyclooxygenase-2 inhibitor component and a glucocorticoid component within an effective time of each respective component, it is preferable to administer both respective components contemporaneously, and more preferable to administer both respective components in a single delivery dose.

In particular, the compositions and pharmaceutical compositions of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions may contain a thickening agent, for example, beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative and flavoring and coloring agents.

The subject combinations can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing of wetting agents and suspending agents which have been mentioned above, or other acceptable agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables.

The subject combination can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and polyethylene glycols.

The novel compositions can also be administered topically, in the form of creams, ointments, jellies, collyriums, solutions or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

The present invention further comprises kits that are suitable for use in performing the methods of treatment, prevention or inhibition described above. In one embodiment, the kit contains a first dosage form comprising a glucocorticoid in one or more of the forms identified above and a second dosage form comprising one or more of the cyclooxygenase-2 inhibitors or prodrugs thereof identified above, in quantities sufficient to carry out the methods of the present invention. Preferably, the first dosage form and the second dosage form together comprise a therapeutically effective amount of the compounds for the prevention or treatment of T cell mediated inflammatory/autoimmune diseases and disorders.

The following examples describe embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis unless otherwise indicated.

### EXAMPLES

General Information

Materials and Methods:

Animal husbandry and plasma sampling: Mice were housed on a 12 h/12 h light/dark cycle with ad libitum access to rodent chow. Blood for measurement of corticosterone and cytokines was obtained by rapid retroorbital phlebotomy into heparinized capillary tubes with a total time from first handling the animal to completion of bleeding not exceeding 30 s. Plasma was separated by centrifugation and stored at −80° C. until assay. Unless otherwise noted, all mice used were 6-10 weeks old and of a C₅₇BL/6×129/Sv genetic background.

For a detailed description of the experimental methods and data provided herein, see Brewer, J. A., The role of glucocorticoids in immune system development and regulation, Dissertation presented to Washington University, St. Louis, Mo. (2002).

Generation of TGRko mice: To build a TGRko targeting vector (pGRloxPneo), a loxP site was inserted into the unique Sac I site in the GR gene region upstream of exon 2 between exons 1B and 1C. A PGKneo (gene encoding for resistance to neomycin) cassette was then subcloned containing flanking loxP sites into intron 2 of the GR gene using oligonucleotide linkers. To obtain ES clones having replaced one copy of the endogenous murine GR locus with the GRloxPneo allele, TC₁ ES cells were electroporated in the presence of linearized pGRloxPneo as has previously been described (See, e.g., Muglia, L. J. et al., J. Clin. Invest., 93:2066-2072 (1994)). DNA from 87 G418 resistant embryonic stem cell (ES) clones was analyzed by Southern blot, employing a probe external to the flanking regions within our targeting vector. Five clones demonstrated homologous recombination of the targeting vector into the endogenous GR locus as evidenced by the appearance of a 4 kb restriction fragment length polymorphism. A clone heterozygous for the floxed exon 2 allele was injected into C₅₇BL/6 blastocysts. After germline transmission, heterozygous mice harboring the floxed exon 2 allele were mated to Lck-Cre transgenic mice. Mice were then mated to homozygosity for the floxed exon 2 allele. Matings were set up between Lck-Cre(+) floxed exon 2 homozygotes and Lck-Cre(−) floxed exon 2 homozygotes to generate Lck-Cre(+) GR^{flox/flox} (referred to as TGRko) and GR^{flox/flox} (referred to as control). Unless otherwise noted, all experiments were performed using age and sex-matched wild-type (WT) and TGRko littermates. (Lck-Cre, refers to the Cre recombinase under control of the Lck proximal promoter).

Antibody detection of GR protein: Total protein was harvested from whole thymus or CD4⁺ thymocytes that had been sorted by flow cytometry (MoFlo®, Cytomation Inc.). Fifteen pg of the protein was resolved on a 4-12% bis-tris polyacrilamide gel, probed with anti-GR anti-sera (M-20, Santa Cruz) at a 1:200 dilution, anti-actin anti-sera (Sigma) at a 1:1000 dilution, and developed using ECL detection reagents (Amersham). Membranes were then stained with Ponceau S solution. (Sigma) to ensure equal loading of protein.

Cytokine Measurements: Plasma cytokines were measured according to manufacturer's instructions (Pharmingen).

Ribonuclease Protection Assay and Microarray Analysis: Total splenic RNA was isolated from WT and TGRko mice eight hours after anti-CD3ε antibody challenge using a RNEasy kit (Qiagen). An RNAse protection assay was then performed on 2 μg of total RNA according to manufacturer's instructions (Pharmingen). Microarray experiments were performed on pooled splenic RNA from the above samples according to manufacturer's instructions (Affymetrix). Data were analyzed using Microarray Suite Version 5.0 (Affymetrix).

Corticosterone Assay: Plasma concentration of corticosterone was determined by RIA (ICN) from blood collected by retroorbital phlebotomy at indicated the timepoints in singly housed adult male mice as previously described. See, e.g., Bethin (2000) ibid.

Flow Cytometry: Thymocytes were dispersed through nylon mesh into PBS, washed, counted on a hemocytometer using trypan blue to exclude non-viable cells, stained for cell surface markers (PE-anti-CD25, PerCP-anti-CD8, APC-anti-CD4, FITC-anti-CD69, PE-anti-TCRβ from PharMingen), washed, resuspended in PBS and analyzed on a FACSCaliber® (Becton Dickinson). Unless otherwise indicated, non-viable cells were excluded from analysis based on forward and side scatter profiles.

Adrenalectomy: Mice were adrenalectomized as previously described and rested one week before being subjected to experimentation. See, e.g., Muglia, L. J. et al, J. Clin. Invest., 105:1269-1277 (2000).

Pharmacologic and antibody treatment: Mice were injected intraperitoneally 100 μg anti-CD3p antibody (145-2C11) diluted in 250 μl PBS. Dexamethasone-treated mice were injected intraperitoneally with 200 μg dexamethasone phosphate just prior to and eight hours after anti-CD3ε antibody challenge. Neutralizing anti-IFNγ antibody (H22, 50 μg) was injected intraperitoneally one day before anti-CD3ε antibody administration as described previously. See, e.g., Ferran, C. et al., Eur. J. Immunol, 21:2349-2353 (1991). Mifepristone (RU 486, Sigma) was dissolved in 100% ethanol (50 mg/ml). Mifepristone-treated mice were given 0.5 mg (diluted in sesame oil) s.c. the night before and one hour prior to inflammatory challenge. Cox-2 selective inhibitor-treated mice were given 300 μg of either SC-236 or NS-398 suspended in PBS/1% Tween-80 or vehicle twice a day for two days by oral gavage as previously described by Gross, G. et al, Am J. Physiol. Regul. Integr. Comp. Physiol., 278:R¹⁴¹⁵-R¹⁴²³ (2000).

Statistical Methods: All results are expressed as mean±SEM unless otherwise stated. Statistical analysis was done by ANOVA with P≦0.05 considered significant.

### Example 1

This example describes the generation of T cell specific glucocorticoid receptor knockout (TGRko) mice.

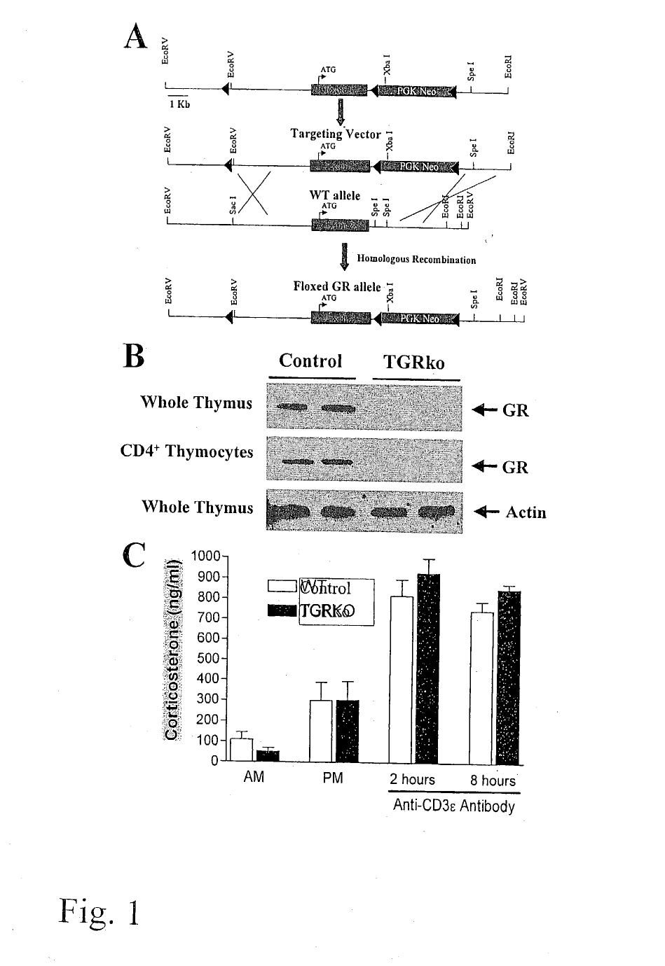

To define specific roles that GR may play in thymocyte development and peripheral T cell activation, specific T cell glucocorticoid receptor (GR) knock out mice were generated by the use of Lck promoter-driven, Cre recombinase-mediated excision of exon 2 of the GR gene (shown in FIG. 1a). While global inactivation of the GR gene results in perinatal lethality due to abnormal lung maturation (See. e.g., Cole, T. J. et al., Genes & Development, 9:1608-1621 (1995)), mice homozygous for the floxed GR gene segment and expression of the Lck-Cre transgene (TGRko mice) were born alive and appeared as healthy as their Lck-Cre (−), but otherwise genetically identical littermates (control mice). Very little GR was noted in whole thymus, and no detectable protein in purified CD4⁺ thymocytes (encompassing both CD4/8⁺ double positive (DP) and CD4⁺ single positive (SP) subpopulations, FIG. 1b). These results indicate that GR is efficiently deleted early in thymocyte development in TGRko mice.

One important function of GR in maintaining normal homeostasis is feedback inhibition of the HPA axis. In this negative feedback loop, adrenally-derived corticosterone acts via the hypothalamus and pituitary to regulate its own production. Additionally, the HPA axis can be regulated by cytokines, neuropeptides and the sympathetic nervous system. See, e.g., Da Silva, J. A., Ann. N Y Acad. Sci. 876:102-117; discussion 117-118 (1999). To determine whether deletion of T cell GR modulates the HPA axis basally, plasma corticosterone levels were analyzed at circadian nadir (morning) and peak (evening). No differences were noted between TGRko and control mice (FIG. 1c). Additionally, TGRko mice mounted a corticosterone response equal to their littermate controls, when challenged with a polyclonal T cell activation stimulus (anti-CD3ε antibody, FIG. 1c). Taken together, these data showed that deletion of GR in the T cell did not alter basal HPA axis function, and indicated that GR signaling in T cells did not alter activation of this axis during an inflammatory response.

In summary, the data that are shown in FIG. 1 indicate that the deletion of T cell glucocorticoid receptor does not alter HPA axis regulation. FIG. 1(A) shows a schematic for targeted deletion of GR exon two. A targeting vector was designed in which exon two was flanked by loxP sites (triangles). Deletion of GR exon two in mice homozygous for the floxed GR allele was mediated by transgenic expression of Cre recombinase controlled by the T cell-specific Lck promoter (TGRko). Littermates homozygous for the floxed GR allele, but not expressing Cre recombinase, served as controls (control). FIG. 1(B) shows total protein, which was extracted from whole thymus or CD4⁺ thymocytes purified by flow cytometry and probed for expression of GR by Western blot analysis. Blots were re-probed for expression of actin as a loading control. FIG. 1(C) shows plasma corticosterone levels, which were measured in TGRko and control mice in the morning, evening, two and eight hours after injection of 100 μg anti-CD3ε antibody (145-2C11, n=4/group).

### Example 2

This example illustrated that T cell glucocorticoid receptor is not required for thymocyte development or peripheral distribution of T lymphocytes.

Previous studies using pharmacologic blockade of steroid biosynthesis in fetal thymic organ culture (FTOC), GR anti-sense transgenic mice, and GR hypomorph alleles have yielded conflicting data on the role of GR in thymocyte development. See, e.g., King, L. B. et al., Immunity, 3:647-656 (1995); Purton, J. F. et al., Immunity, 13:179-186 (2000). To determine whether GR signaling affects thymocyte development in mice with T cell-specific GR deletion, thymocytes were analyzed from 8-10 week old sex-matched TGRko and control mice. No significant difference was noted in total thymus cellularity or subset distribution between genotypes (Total cells (×10⁶): TGRko=85.8±10.5, n=13; control=102.5±11.6, n=14). Additionally TCRβ and CD25 surface expression did not differ between TGRko and control thymocytes (data not shown). In concert with these findings, there was a normal distribution of T cells in spleen and lymph nodes of TGRko mice. These data indicated that GR is not required for T cell development.

### Example 3

This example shows that T cell glucocorticoid receptor is required for prevention of lethality and downregulation of multiple cytokines after T cell activation.

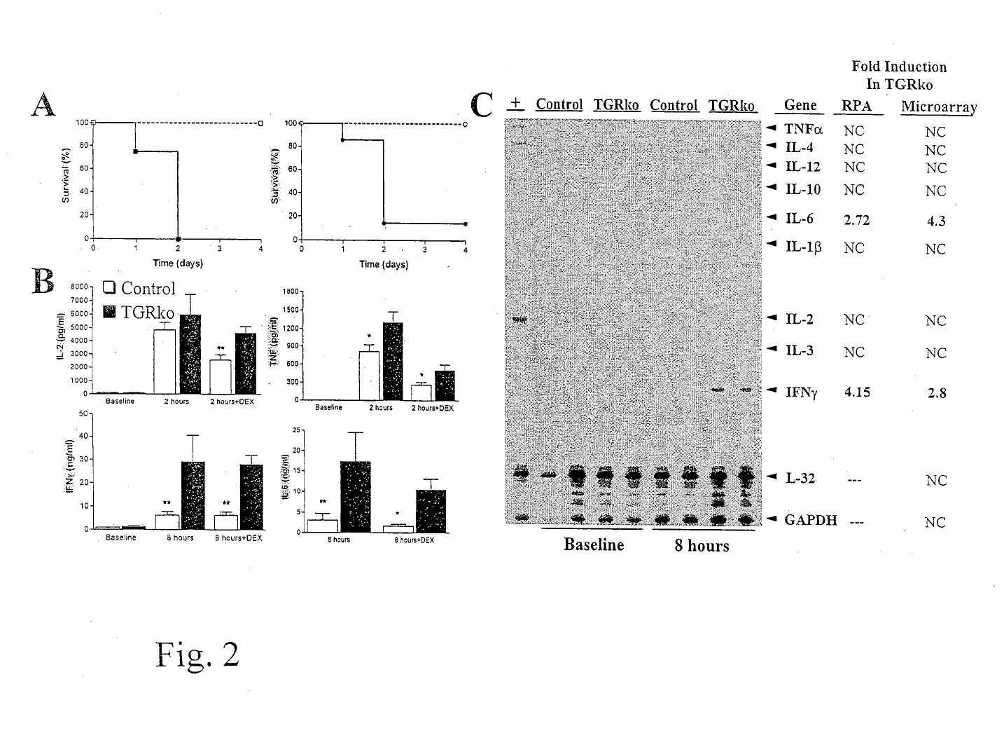

To evaluate regulation of cytokines and other pro-inflammatory molecules by T cell GR, anti-CD3ε antibodies were administered to TGRko and control mice. In the spleen, this polyclonal T cell activation stimulus has been shown to induce rapid, but transient transcription of IL-2, IL-3, IL-4, IL-6, IFNγ and TNFα, leading to measurable, but ephemeral plasma levels of these cytokines between one and eight hours after administration. (See, e.g., Scott, D. E. et al., J. Immunol., 145:2183-2188 (1990). In mice and humans, this leads to an acute, but self-limited clinical syndrome characterized by hypotension, hypomotility, fever and hypoglycemia, which can be modulated by pharmacologic GC administration. See, Charpentier, B. et al., Transplantation, 54:997-1001 (1992); Ferran, C. et al., Transplantation, 50:642-648 (1990). In contrast to uniform survival in control mice, high mortality was noted in TGRko mice after anti-CD3ε antibody administration, which could not be rescued by pre-treatment with the synthetic GC dexamethasone (DEX, FIG. 2a). These results suggested that the T cell is a critical target for GC down-regulation of immune responses.

To determine whether TGRko mice were dying from altered cytokine regulation, plasma cytokine levels were measured after polyclonal T cell activation. Significant increases in TNFα, IFNγ, and IL-6, but not IL-2, were noted in TGRko mice after T cell activation (FIG. 2b). Interestingly, DEX administration reduced plasma levels of TNFα in both TGRko and control mice, but had no significant effect on plasma IL-2 in TGRko mice. Plasma IFNγ was not affected by DEX administration in either genotype (FIG. 2b). These data suggest that in contrast to other cytokines, IFNγ regulation is specifically controlled by T cell GR signaling.

To determine the role of endogenous T cell GR signaling in transcriptional regulation of these and other inflammatory genes, gene expression in spleens of TGRko and control mice were compared eight hours after T cell activation by microarray analysis and ribonuclease protection assay (RPA). Of 21 known genes that were induced 2.5 fold or greater in microarray analysis of TGRko compared to control splenocytes, 10 have documented immune function. For example, a listing of genes (with fold induction values in parenthesis) shows: T cell and activation regulated chemokine (7.0), small inducible cytokine B subfamily, member 5 (6.1), IL-6 (4.3), COX-2 (3.5), Src-suppressed C kinase substrate (3.5), MMP-1 (3.2), Eotaxin precursor (3.2), IFNγ (2.8), SOCS-3 (2.6), protein kinase inhibitor (2.6). Providing validation of these data, expression of 9/9 cytokines from the same samples, as well as from pooled lymph nodes (data not shown), analyzed by RPA showed the same degree of induction (or lack thereof) as that shown by microarray (FIG. 2c).

Interestingly, IFNγ, but not TNFα or IL-2 was elevated in TGRko microarray and RPA samples (IL-6 was induced in one of two RPA samples). The low levels of IL-2 and TNFα RNA correlated with reduced plasma cytokine measurements eight hours after stimulation (IL-2 and TNFα were not statistically different in TGRko and control mice, data not shown). These data suggest that endogenous GCs acting through T cell GR are required for IFNγ transcriptional suppression (possibly mediated through inhibition of Stat4 phosphorylation (See, e.g., Franchimont, D. et al., J. Immunol., 164:1768-1774 (2000)), but not for TNFα or IL-2.

In summary, the data shown in FIG. 2 indicate that T cell GR is required for prevention of lethality and downregulation of multiple cytokines after activation. (A) Survival presented as a Kaplan-Meyer plot (□ control, n=10; ▪ TGRko, n=4; ◯ control+DEX, n=10; ● TGRko+DEX, n=7). No further mortality was noted after four days. P<0.01 between TGRko and control mice in both plots (B) Plasma cytokine levels were measured in TGRko and control mice two and eight hours after injection of anti-CD3ε antibody (100 μg)±dexamethasone administration (200 μg 1 hour before and 8 hours after anti-CD3ε antibody) by ELISA (n=6-9/group for TGRko, 8-13/group for control mice). (C) Splenic RNA (2 μg) from TGRko and control mice was analyzed by RPA at baseline or eight hours after injection of anti-CD3ε antibody ((+) denotes positive control RNA provided by the manufacturer). Expression was normalized to GAPDH and quantitated using a phosphorimager. The same samples were pooled and analyzed by microarray. *, P<0.05, **, P<0.01 between TGRko and control.

### Example 4

This example shows that overproduction of IFNγ was not the cause of mortality in T cell activated TGRko mice.

To determine if unchecked IFNγ production was inducing a high degree of mortality in TGRko mice, neutralizing anti-IFNγ antibodies were administered to the mice before in vivo T cell stimulation. Neutralizing anti-IFNγ antibodies decreased the level of plasma IFNγ below the limit of detection (624 pg/ml), however, there was no reduction in mortality (3/3 mice died at 1.67±0.44 days). These results suggested that although GR signaling in T cells is important in the regulation of IFNγ production, this dysregulation does not directly result in the high mortality seen in TGRko mice after T cell activation.

### Example 5

This example shows that Cox-2 dysregulation is directly involved in the mortality of TGRko mice and indicates the efficacy of the administration of a Cox-2 selective inhibitor to reduce the mortality.

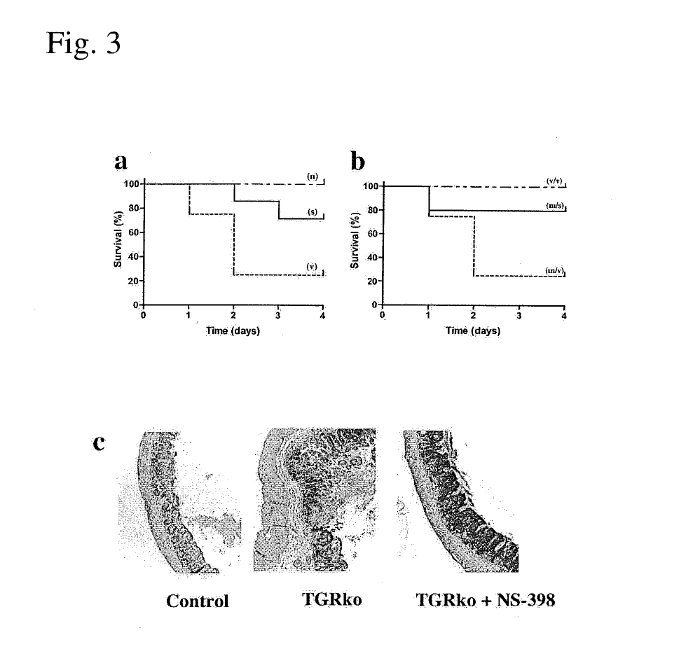

Glucocorticoids have been shown to regulate expression of pro-inflammatory mediators in addition to cytokines. Of note, cyclooxygenase 2 (Cox-2) was discovered as a GC-modulated enzyme that was induced in monocytes after LPS administration, and subsequently has been shown to be induced in vitro in T cells after activation. See, e.g., Iniguez, M. A. et al, J. Immunol., 163:111-119 (1999); Masferrer, J. L. et al., Proc. Natl. Acad. Sci. USA, 89:3917-3921 (1992). In this work, it was found that Cox-2 mRNA levels were 3.5 fold higher in TGRko spleenocytes compared to controls eight hours after anti-TCRε antibody administration by microarray analysis. To determine whether Cox-2 dysregulation was directly involved in induction of mortality by polyclonal T cell activation in TGRko mice, mice were treated with each of two selective Cox-2 inhibitors (SC-236, which was provided by the Pharmacia Company, and NS-398, available from Cayman Chemical, Ann Arbor, Mich.) before and after anti-CD3ε antibody administration. Protection from lethality was noted in TGRko mice treated with either SC-236 (s) or NS-398 (n) compared to vehicle-treated mice (v) (FIG. 3a).

To bring these observations into the more physiologically and clinically relevant context of global GC deficiency/resistance, these experiments were repeated in control mice pre-treated with mifepristone (RU-486), a GR antagonist. Highlighting the importance of GR signaling in this system, mice treated with mifepristone+vehicle (m/v) were significantly more susceptible to the lethal affects of T cell activation compared to vehicle controls (v/v) (FIG. 3b). As with TGRko mice treated with SC-236, control mice treated with mifepristone+SC-236 (m/s) (were significantly protected compared to their vehicle-treated counterparts (FIG. 3b). Additionally, SC-236 treatment of adrenalectomized control mice showed essentially the same degree of rescue, though on a shorter timescale (0/3 Sham operated+vehicle, 2/6 ADX+SC-236, and 4/5 ADX+vehicle mice died within eight hours of anti-CD3ε antibody treatment). Taken together, these data directly show that endogenous T cell GR modulation of Cox-2 expression is required to prevent polyclonal T cell activation from becoming lethal. Histological analysis in the cecum in mice treated with anti-CD3ε antibody demonstrated marked edema, inflammation, and mucosal disruption in the TGRko mice and rescue with Cox-2 inhibition with NS-398. See, FIG. 3C. These sections are representative of 3-5 mice per group analyzed.

In summary, the data shown in FIG. 3 show that cyclooxygenase 2 inhibition protects against mortality induced by polyclonal T cell activation in GR-deficient mice. (A) TGRko were treated with SC-236, (solid line (s), n=7), NS-398 (alternately dashed line (n)), or vehicle (dashed line (v), n=3) one hour before anti-CD3ε antibody administration, and twice a day for two days thereafter. (B) Control mice were treated with mifepristone+SC-236 (m/s, solid line, n=10), mifepristone+vehicle (m/v, dashed line, n=8), or vehicle+vehicle (v/v, mixed line, n=3) as described in materials and methods. P<0.05 between SC-236 and vehicle treated mice.

### Example 6

This illustrates the production of a composition containing parecoxib sodium and dexamethasone sodium phosphate, and of a pharmaceutical composition containing the combination.

Parecoxib sodium can be produced according to the procedures described in U.S. Pat. No. 5,932,598. Dexamethasone sodium phosphate for injection can be obtained from Merck, Wyeth-Ayerst, and other suppliers, under the trade name “Decadron”.

A therapeutic composition of the present invention can be formed by intermixing parecoxib sodium (40 g) into 187.5 ml of dexamethasone sodium phosphate sterile injection solution (containing 0.75 g of dexamethasone phosphate, 1.5 g of creatinine, 1.875 g sodium citrate, sodium hydroxide to adjust pH, and water for injection q.s., with 187.5 mg sodium bisulfite, 281 mg methylparaben, and 37.5 mg propylparaben as preservatives; available as Decadron Phosphate injection, 4 mg/ml, from Merck & Co., Inc., Whitehouse Station, N.J.). Additional water may be added if necessary for the complete dissolution of all solid components.

After mixing, the combination of parecoxib and dexamethasone form a therapeutic composition that is sufficient for the production of about 1000 human single dose units. Each single dose unit contains about 40 mg of parecoxib sodium and about 0.75 mg of dexamethasone phosphate.

Therapeutic and pharmaceutical compositions comprising a combination of any of the cyclooxygenase-2 inhibitors and any of the glucocorticoids that are described above can be formed by similar methods.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinency of the cited references.

In view of the above, it will be seen that the several advantages of the invention are achieved and other advantageous results obtained.

As various changes could be made in the above methods and compositions without departing from the scope of the invention, it is intended that all matter contained in the above description shall be interpreted as illustrative and not in a limiting sense.

What is claimed is:

1. A method of preventing or treating a T cell mediated inflammatory/autoimmune disease or disorder in a subject having a glucocorticoid regulation deficiency, where the subject is in need of such treatment, the method comprising administering to the subject an effective amount of a cyclooxygenase-2 inhibitor or prodrug thereof.

2. A method of preventing or treating morbidity and mortality associated with T cell activation in a subject having a glucocorticoid regulation deficiency, the method comprising administering to the subject an effective amount of a cyclooxygenase-2 inhibitor.

3. A method of limiting morbidity and mortality in a subject having a glucocorticoid regulation deficiency, the method comprising administering to the subject an effective amount of a cyclooxygenase-2 inhibitor prior to, during, or after the subject has undergone a T cell activating process.

4. A method of treating a subject for a T cell mediated inflammatory/autoimmune disease or disorder, the method comprising administering an effective amount of a cyclooxygenase-2 inhibitor to a subject having a glucocorticoid regulation deficiency after the subject has undergone T cell activation process.

5. The method according to claim 1, wherein the T cell activating process comprises the contact of a T cell of the subject with a T cell activating agent.

6. The method according to claim 5, wherein the T cell activating agent is selected from the group consisting of a T cell activating antigen and a T cell specific activating antibody.

7. The method according to claim 1, wherein the subject is a vertebrate.

8. The method according to claim 7, wherein the subject is a human.

9. The method according to claim 1, wherein the glucocorticoid regulation deficiency comprises one that is due to a glucocorticoid insufficiency, a glucocorticoid resistance, or an overwhelming T cell activating stimulus.

10. The method according to claim 9, wherein the glucocorticoid regulation deficiency comprises a glucocorticoid insufficiency.

11. The method according to claim 10, wherein the glucocorticoid insufficiency is due to Addison's disease, idiopathic atrophy of the adrenal cortex, destruction of the adrenal gland, removal of the adrenal gland, presence of a drug that blocks steroid synthesis, a glucocorticoid receptor insufficiency, a glucocorticoid production insufficiency, or a combination thereof.

12. The method according to claim 9, wherein the glucocorticoid regulation deficiency comprises a glucocorticoid resistance.

13. The method according to claim 12, wherein the glucocorticoid resistance is due to chronic exogenous glucocorticoid treatment, chronic inflammatory stimulus, an abnormally low GRα/GRβ ratio, chronic T cell mediated inflammatory disease, or chronic T cell mediated autoimmune disease.

14. The method according to claim 9, wherein the glucocorticoid regulation deficiency is due to the subject experiencing an overwhelming T cell activating stimulus.

15. The method according to claim 14, wherein the overwhelming T cell activating stimulus is selected from the group consisting of graft vs. host disease, toxic shock syndrome, bacterial sepsis, viral sepsis, superantigen mediated food poisoning, transplant rejection, immunosuppression using anti-CD3 antibodies or equivalent, multiple sclerosis, systemic lupus erythematosus, rheumatoid arthritis, and inflammatory bowel disease.

16. The method according to claim 1, wherein the effective amount comprises a therapeutically effective amount.

17. The method according to claim 1, wherein the effective amount comprises an amount sufficient to prevent, reduce, or alleviate the signs and symptoms caused by T cell activation in the subject, or to retard or prevent disease progression.

18. The method according to claim 1, wherein the cyclooxygenase-2 inhibitor is selected from the group consisting of cyclooxygenase-2 inhibiting: indoles, naphthylalkanones, oxicams, para-aminophenol derivatives, propionic acids, salicylates, fenamates, pyrazoles, nitric oxide-releasing nonsteroidal anti-inflammatory drugs, and misoprostol combinations with nonsteroidal anti-inflammatory drugs.

19. The method according to claim 18, wherein the cyclooxygenase-2 inhibitor is selected from the group consisting of etodolac, indomethacin, sulindac, tolmetin, nabumetone, piroxicam, fenoprofen, flurbiprofen, ibuprofen, ketoprofen, naproxen, naproxen sodium, oxaprozin, aspirin, choline magnesium trisalicylate, diflunisal, meclofenamic acid, mefenamic acid, and phenylbutazone.

20. The method according to claim 1, wherein the cyclooxygenase-2 inhibitor is a cyclooxygenase-2 selective inhibitor or prodrug thereof which has a cyclooxygenase-2 IC₅₀ of less than about 0.2 μmol/L.

21. The method according to claim 20, wherein the cyclooxygenase-2 selective inhibitor is selected from the group consisting of celecoxib, valdecoxib, deracoxib, rofecoxib, etoricoxib, parecoxib, lumiracoxib, meloxicam, SD-8381, ABT-963, BMS-347070, NS-398, prodrugs of any of them, and mixtures thereof.

22. The method according to claim 21, wherein the cycloxygenase-2 selective inhibitor comprises a compound selected from the group consisting of celecoxib, valdecoxib, parecoxib, prodrugs of any of them, and mixtures thereof.

23. The method according to claim 1, wherein the cyclooxygenase-2 inhibitor is administered with a glucocorticoid.

24. The method according to claim 23, wherein the glucocorticoid is selected from the group consisting of synthetic glucocorticoids, natural glucocorticoids, non-steroidal glucocorticoid mimics that are not dissociated, steroidal glucocorticoid analogs that are dissociated, and non-steroidal glucocorticoid mimics that are dissociated.

25. The method according to claim 24, wherein the glucocorticoid is selected from the group consisting of mometasone, fluticasone, budesonide, betamethasone, prednisolone, methylprednisolone, dexamethasone, hydrocortisone (cortisol), triamcinolone, cortisone, corticosterone and prednisone.

26. The method according to claim 1, wherein the cyclooxygenase-2 inhibitor comprises a material that is selected from the group consisting of celecoxib, valdecoxib, deracoxib, rofecoxib, etoricoxib, parecoxib, lumiracoxib, meloxicam, SD-8381, ABT-963, BMS-347070, NS-398, prodrugs of any of them, and mixtures thereof, and wherein the cyclooxygenase-2 inhibitor is administered to the subject in combination with a glucocorticoid that is selected from the group consisting of mometasone, fluticasone, budesonide, betamethasone, prednisolone, methylprednisolone, dexamethasone, hydrocortisone (cortisol), triamcinolone, cortisone, corticosterone and prednisone.

27. The method according to claim 26, wherein the cyclooxygenase-2 selective inhibitor is selected from the group consisting of celecoxib, valdecoxib, parecoxib, rofecoxib and etoricoxib and the glucocorticoid is selected from the group consisting of dexamethasone, hydrocortisone, betamethasone, methylprednisolone, prednisolone, and prednisone.

28. The method according to claim 26, wherein the cyclooxygenase-2 inhibitor is a cyclooxygenase-2 selective inhibitor and the weight ratio of the amount of cyclooxygenase-2 selective inhibitor or prodrug thereof to the amount of glucocorticoid that is administered to the subject is within a range of from about 0.03:1 to about 35,000:1.

29. The method according to claim 28, wherein the weight ratio of the amount of cyclooxygenase-2 selective inhibitor or prodrug thereof to the amount of the glucocorticoid that is administered to the subject is within a range of from about 0.5:1 to about 100:1.

30. A composition for the prevention and/or treatment of T cell mediated inflammatory/autoimmune diseases and disorders in a subject having a glucocorticoid regulation deficiency, the composition comprising a combination of a cyclooxygenase-2 inhibitor and a glucocorticoid.

31. The composition according to claim 30, wherein the cyclooxygenase-2 inhibitor is a cyclooxygenase-2 selective inhibitor.

32. The composition according to claim 31, wherein the cyclooxygenase-2 selective inhibitor and the glucocorticoid are present each in an amount sufficient to provide an effective amount of the combination.

33. A pharmaceutical composition for the prevention and/or treatment of T cell mediated inflammatory/autoimmune diseases and disorders in a subject having a glucocorticoid regulation deficiency, the pharmaceutical composition comprising a pharmaceutically acceptable excipient and a combination of a cyclooxygenase-2 inhibitor and a glucocorticoid.

34. The pharmaceutical composition according to claim 33, wherein the cyclooxygenase-2 inhibitor comprises a cyclooxygenase-2 selective inhibitor.

35. A kit for the prevention and/or treatment of T cell mediated inflammatory/autoimmune disease or disorder in a subject having a glucocorticoid regulation deficiency, the kit comprising one dosage form comprising a cyclooxygenase-2 inhibitor and a second dosage form comprising a glucocorticoid, wherein the cyclooxygenase-2 inhibitor and a glucocorticoid are present each in an amount sufficient that the kit provides an effective amount of the combination.

36. The kit according to claim 35, wherein the cyclooxygenase-2 inhibitor is a cyclooxygenase-2 selective inhibitor.
